(19) United States
(12) Patent Application Publication
(10) Pub. No.: US 2020/0095299 A1
(43) Pub. Date: Mar. 26, 2020

(54) STEM CELL MODEL OF APOE GENOTYPE AND Abeta42-DEPENDENT NEURODEGENERATION AND METHODS OF USING THE SAME

(71) Applicant: CITY OF HOPE, Duarte, CA (US)

(72) Inventors: Paul M. SALVATERRA, Pasadena, CA (US); Jiing-Kuan YEE, Arcadia, CA (US)

(21) Appl. No.: 16/512,278

(22) Filed: Jul. 15, 2019

Related U.S. Application Data
(60) Provisional application No. 62/698,039, filed on Jul. 14, 2018.

(51) Int. Cl. C07K 14/47; C12N 5/0793; G01N 33/50

(52) U.S. Cl. CPC C07K 14/4711; C12N 5/0619; C12N 2502/081; C12N 2506/02; G01N 33/5058

(57) ABSTRACT

Provided is an isolated cell including a modified amyloid beta precursor protein (APP) gene. The modified APP gene encodes a secretory peptide, and the secretory peptide is Amyloid Beta₁₋₄₀ (Aβ₁₋₄₀) or Amyloid Beta₁₋₄₂ (Aβ₁₋₄₂). The isolated cell can additionally have a modified Apolipoprotein E (APOE) gene, and/or at least one marker. Also provided is an in vitro model including at least one population of cells having a modified APP gene encoding a secretory peptide such as Aβ₁₋₄₀ or Aβ₁₋₄₂. The population of cells is subjected to at least one differentiation protocol. Further provided is a method of screening treatments including contacting at least one population of cells disclosed herein with at least one agent and determining if the agent has an effect on phenotype. The agent is a drug, a salt, a mineral, an antibody, a humanized antibody, an enzyme, a protein, a peptide, a cell, a modified cell, a stem cell, a plant-based substance, a plant derivative, an antioxidant, or an antioxidant derivative.

### CROSS REFERENCE TO RELATED APPLICATIONS

This application claims priority to U.S. Provisional Patent Application No. 62/698,039, filed Jul. 14, 2018, which is incorporated herein by reference in its entirety.

### SEQUENCE LISTING

This application contains a Sequence Listing, which was submitted in ASCII format via EFS-Web, and is hereby incorporated by reference in its entirety. The ASCII copy, created on Oct. 17, 2019, is named 8148US02_SequenceListing.txt and is 10 KB in size.

### BACKGROUND

Of the three leading causes of death (cardiovascular disease, cancer and neurodegenerative disorders), Alzheimer's disease (AD) is the only one still increasing in incidence. People now live longer, and age is the most prominent demographic risk factor for AD. The lack of proper cellular models for the primary cellular phenotype of AD: neurodegeneration (ND) limits the ability to use cell culture systems to test therapeutic agents designed to prevent ND as well as design rational evidence-based stem cell clinical trials. Further, existing transgenic mouse models have failed to recapitulate the ND characteristic of AD patients. Accordingly, a better model is needed.

The pathological and behavioral identification of AD more than 100 years ago, in combination with more recent intensive research efforts, have not led to significant progress in treatment or prevention. Many key aspects of the pathology and underlying disease mechanisms remain unknown and are continually debated. This uncertainty exists, at least in part, due to deficiencies in AD models. Currently, no model accurately reflects the full complement of disease phenotypes, in particular, chronic progressive ND (Zahs & Ashe 2010). Many AD-associated phenotypes have been established, but the cause and effect relationships between these phenotypes and ND remain largely unresolved. Further complexity results from the heterogeneous mixture of cell types in brain tissue and their potential involvement AD.

Amyloid Beta₁₋₄₀ (Aβ₁₋₄₀) and Amyloid Beta₁₋₄₂ (Aβ₁₋₄₂) are the major peptides generated during amyloidogenic processing amyloid beta precursor protein (APP). The Aβ₁₋₄₂ peptide is believed to be the toxic agent responsible for initiating the pathogenic cascade that ultimately results in neuronal death and that Aβ₁₋₄₂ accumulation is a driver of pathogenic changes (Musiek & Holtzman 2015). However, its mechanism of pathogenesis is not completely understood. Moreover, a host of non-Aβ₁₋₄₂ environmental and genetic factors influence AD contraction risk and progression rate. The primary genetic risk factor is apolipoprotein E (APOE) allele type. APOE codes for a 299 amino acid lipoprotein. Individuals with the rare APOE2 allele are less likely to develop AD. By contrast, individuals with even a single E4 allele have a much higher risk, even reaching semi-dominance in APOE4/E4 homozygotes (Yu et al. 2014).

Previous AD clinical trials have failed. These trials were primarily developed using preclinical data from transgenic mouse models. The most commonly used mouse models depend on expression of mutant forms of one or more familial AD causative genes (App, Psen1, or Psen2). Similar to AD patients, these animals accumulate Aβ₁₋₄₂ peptide via amyloidogenic processing of App. While mouse models have been useful in establishing certain AD features, nearly all do not exhibit robust chronic progressive ND.

There is a need for model systems that can be used to exhibit ND since these can then be used to screen for agents and factors to prevent cell death. Existing studies linking Aβ₁₋₄₂ to ND include rodent and Drosophila models that directly express Aβ₁₋₄₂ in neurons, rather than APP, as is common in most mouse AD models. These direct Aβ₁₋₄₂ models lead to plaque-like amyloid deposition and robust progressive ND (Ling et al. 2009; Lewis et al. 2001; McGowan et al. 2005; LaFerla et al. 1995; Abramowski et al. 2012). In both vertebrate and invertebrate direct expression models, Aβ₁₋₄₂ must be routed through the normal cellular secretory membrane trafficking pathway to result in ND. While direct expression models are criticized as “not really AD”, they represented an improvement over APP processing models for the following reasons. First, downstream changes are not complicated by the time or the cellular site of Aβ₁₋₄₂ generation following APP processing. Second, if Aβ₁₋₄₂ initiates a pathogenic cascade similar to AD—a reasonable assumption based on numerous mouse and Drosophila direct expression models—the buildup of sufficient levels of proteotoxic peptide should be faster in direct expression models. Finally, certain embodiments described herein represent isogenic cell lines. Isogenic cells are characterized as having the same background genotype and differ only in the single modified gene being tested. This feature eliminates complexities in genetic background which complicate phenotypic comparisons in many types of AD patient investigations or other types of cellular models such as patient derived neurons. However, it is well known that a host of genes not directly involved in Aβ₁₋₄₂ production or removal have significant—although quantitatively small—risk effects for AD. The statistical power of most human genetic studies, however, is confounded by uncontrolled genetic variance.

Human cells offer important advantages over existing animal models as a screening platform for preclinical testing of AD therapeutic agents and/or treatment regimens. For example, working with human cells obviates species differences (both known and unknown) that affect cellular and molecular processes important for ND. But, current cell models have failed to produce the desired ND phenotype. Notable advances have been made studying patient-derived induced pluripotent stem cells (Israel & Goldstein 2011; Yagi et al. 2011). These cells exhibit some AD-associated phenotypes including increased Aβ₁₋₄₂ accumulation. But, these cells do not exhibit ND. An alternative human cell model overexpresses mutant APP and PSEN1 in human neural stem cells (Choi et al. 2014). Following differentiation, these cells form extracellular amyloid plaques and intracellular neurofibrillary tangles. But again, that model lacks the ND phenotype.

Accordingly, there exists a need for an AD model that exhibits ND and also accurately represents the behavior of human cells for use in applications such as in vitro systems and as screening mechanisms for potentially therapeutic agents and/or treatment regimes.

### SUMMARY

In a first embodiment, an isolated stem cell is modified to directly express Aβ₁₋₄₂ rather than APP. In this embodiment, one allele of the normal APP gene is modified such that a secretory form of Aβ₁₋₄₂ is produced in place of APP. In this embodiment, expression from this modified allele does not depend on amyloidogenic processing of APP to generate proteotoxic Aβ₁₋₄₂. In this embodiment, the Aβ₁₋₄₂ peptide is under the control of normal APP regulatory DNA and the peptide product is routed through the normal secretory pathway. In a second embodiment, an isolated stem cell is modified to directly express a secretory form of Aβ₁₋₄₀ (rather than Aβ₁₋₄₂ as in the first embodiment). Aβ₁₋₄₀ is the major product of APP amyloidogenic processing, but is much less toxic than Aβ₁₋₄₂, even though it is produced at higher levels in AD.

In another embodiment, stem cells from the first and second embodiments may be additionally modified to alter APOE genotype. In one embodiment, the APOE genotype is modified to APOE2/E2. In another embodiment, the APOE genotype is modified to APOE3/E3. In another embodiment, the APOE genotype is modified to APOE4/E4.

In one embodiment, the modified cells described herein may display a marker. In some embodiments, the marker may be a cell type marker. In some embodiments, the cell type marker may be a neuronal marker. In some embodiments, the cell type marker may be an astrocyte marker. In other embodiments, the cell type marker may be an astrocyte marker. In other embodiments, the marker may be a fluorescent marker. In these embodiments, the fluorescent marker may be any suitable fluorescent marker. Suitable fluorescent markers may include, for example, green fluorescent protein (GFP), red fluorescent protein (RFP), cyan fluorescent protein (CFP), yellow fluorescent protein (YFP), mCherry fluorescent protein (mCherry), Aequorea coerulescens GFP (AcGFP1), DsRed-Monomer fluorescent protein (DsRed-Monomer), tdTomato fluorescent protein (tdTomato), Discosoma sp. red fluorescent protein (DsRed), DsRed-Express fluorescent protein (Ds-Red-Express), or E2-Crimson fluorescent protein (E2-Crimson).

In one embodiment, an in vitro model is generated by subjecting a population of modified stem cells as described in the foregoing embodiments to a differentiation protocol. In one embodiment, the differentiation protocol is designed to favor production of cholinergic motor neurons (ChAT+/Tuj+). In another embodiment, the differentiation protocol is designed to obtain CNS non-limb innervating ChAT positive neurons.

In one embodiment, an in vitro model is generated from a population of modified stem cells described herein, the population of modified stem cells having a modified APP gene, wherein one allele of the modified APP gene encodes a secretory peptide and the secretory peptide is Aβ₁₋₄₂ or Aβ₁₋₄₀. In another embodiment, a population of modified stem cells encoding the secretory peptide Aβ₁₋₄₂ or Aβ₁₋₄₀ has been further modified such that their APOE genotype is APOE2/E2. In another embodiment, a population of modified stem cells encoding the secretory peptide Aβ₁₋₄₂ or Aβ₁₋₄₀ has been further modified such that their APOE genotype is APOE3/E3. In another embodiment, a population of modified stem cells encoding the secretory peptide Aβ₁₋₄₂ or Aβ₁₋₄₀ has been further modified such that their APOE genotype is APOE4/E4.

In one embodiment, an in vitro model is generated from a population of modified stem cells having a modified APP gene, wherein the modified APP gene encodes a secretory peptide and the secretory peptide is Aβ₁₋₄₂ or Aβ₁₋₄₀. In another embodiment, a population of modified stem cells encoding the secretory peptide Aβ₁₋₄₂ or Aβ₁₋₄₀ have undergone a differentiation protocol that favors generating cholinergic motor neurons (ChAT+/Tuj+). In another embodiment, a population of modified stem cells encoding the secretory peptide Aβ₁₋₄₂ or Aβ₁₋₄₀ have undergone a differentiation protocol that favors generating CNS non-limb innervating ChAT+neurons. In another embodiment, a population of modified stem cells encoding the secretory peptide Aβ₁₋₄₂ or Aβ₁₋₄₀ have undergone a differentiation protocol that favors generating astrocytes. In another embodiment, a population of modified stem cells encoding the secretory peptide Aβ₁₋₄₂ or Aβ₁₋₄₀ have undergone a differentiation protocol that favors generating microglial cells. Additional embodiments will differentiate stem cells into various types of cortical neurons known to be at special risk of ND in Alzheimer's disease.

In one embodiment, an in vitro model is generated from a population of modified stem cells having a modified APP gene, wherein the modified APP gene encodes a secretory peptide and the secretory peptide is Aβ₁₋₄₂ or Aβ₁₋₄₀. In another embodiment, the modified stem cells encoding the secretory peptide Aβ₁₋₄₂ or Aβ₁₋₄₀ have at least one marker. In some embodiments, the marker is a fluorescent marker. In some embodiments, the fluorescent marker is GFP. In some embodiments, the fluorescent marker is RFP, CFP, YFP, mCherry, AcGFP1, DsRed-Monomer, tdTomato, DsRed, DsRed-Express, E2-Crimson, or any other suitable fluorescent protein. In some embodiments, the marker is at least one cell type marker. In some embodiments, the marker is a neuronal marker. In some embodiments, the marker is an astrocyte marker. In some embodiments, the marker is a microglial marker. In some embodiments, the marker is a lineage marker. In some embodiments, the marker is a cell type marker that is also fluorescent. In some embodiments, the marker is a lineage marker that is also fluorescent.

In one embodiment, an in vitro model is generated by co-culturing modified stem cells having a modified APP gene, wherein the modified APP gene encodes a secretory peptide and the secretory peptide is Aβ₁₋₄₂ or Aβ₁₋₄₀ with cells from another source. In one embodiment, an in vitro model is generated by co-culturing modified stem cells having a modified APP gene that have been subjected to a differentiation protocol with cells from another source. In one embodiment, the population of modified stem cells have a modified APP gene, wherein the modified APP gene encodes a secretory peptide and the secretory peptide is Aβ₁₋₄₂ or Aβ₁₋₄₀. In certain embodiments, the differentiation protocol favors generating cholinergic neurons (ChAT+/Tuj+), CNS non-limb innervating ChAT+neurons, astrocytes, or any other type of neuron at risk in AD. In one embodiment, modified stem cells that have undergone a differentiation protocol that favors generating cholinergic neurons (ChAT+/Tuj+) are co-cultured with modified stem cells that have undergone a differentiation protocol that favors generating astrocytes. For example, in one embodiment, modified stem cells that have undergone a differentiation protocol that favors generating CNS non-limb innervating ChAT+neurons are co-cultured with modified stem cells that have undergone a differentiation protocol that favors generating astrocytes. In another embodiment, modified stem cells that have at least one marker are co-cultured with cells from another source. In one embodiment, the co-cultured modified stem cells have a neuronal marker. In one embodiment, the co-cultured modified stem cells have an astrocyte marker. In other embodiments, the co-cultured modified stem cells have a microglial marker. In other embodiments, the co-cultured modified stem cells have at least one lineage marker. In other embodiments, the co-cultured modified stem cells have more than one marker. In other embodiments, the co-cultured modified stem cells have at least one fluorescent marker. In other embodiments co-cultured cells may be microglia either alone or in combination with astrocytes.

In one embodiment, an in vitro population of modified stem cells is contacted with at least one agent to determine whether the agent has an effect on ND. In one embodiment, an in vitro population of modified stem cells is contacted with at least one agent to determine whether the agent has an effect on synapse number. In one embodiment, the agent is a drug. In another embodiment, the agent is a salt. In another embodiment, the agent is an antibody. In another embodiment, the agent is an enzyme. In another embodiment, the agent is a protein. In another embodiment, the agent is a peptide. In another embodiment, the agent is a cell. In another embodiment, the agent is a modified cell. In another embodiment, the agent is plant-based. In another embodiment, the agent is an antioxidant. In one embodiment, the treatment includes at least one stem cell therapy. In another embodiment, the treatment includes at least agent and at least one stem cell therapy. In another embodiment, the treatment includes a plurality of agents. In another embodiment, the treatment includes a plurality of agents and at least one stem cell therapy. In another embodiment, the treatment includes a plurality of agents and a plurality of stem cell therapies.

### BRIEF DESCRIPTION OF THE DRAWINGS

This application contains at least one drawing executed in color. Copies of this application with color drawing(s) will be provided by the Office upon request and payment of the necessary fees.

FIGS. 1A-C: FIG. 1A is a schematic diagram illustrating TALEN editing of the H9 APP locus where TALEN pairs induced a double strand break in the 1st exon; homology-directed repair inserted a cassette containing Aβ₁₋₄₂ or Aβ₁₋₄₀ coding sequence preceded by a secretory signal; and the insert is flanked by homology arms and includes a drug selection gene, and a poly A signal. The signal peptide is on the left and the puro/polyA site is on the right of the Aβ fragment. FIG. 1B is a schematic diagram of TALEN editing strategy. FIG. 1C shows the details of genomic editing of APP gene locus. TALEN pairs were designed to target and induce a double strand break (DSB) in the first exon upstream of the normal APP translation initiation codon (APP ATG). The DSB was repaired by homologous recombination in the presence of plasmids containing the coding sequence for either Aβ40 or Aβ42 fused in frame with a rat preproenkephalin secretory signal sequence (SS) and followed by a polyA tail. Repair plasmids additionally included a PGK puromycin drug selection gene (Puro) and were flanked by left and right homology arms homologous to APP flanking sequences (HAL, HAR). Cassette insertions were confirmed by genomic PCR using specific primers in either the HAL (5′) or the HAR (3′) and a site in the insertion cassette. This editing strategy simultaneously inactivates one APP allele and replaces it with a cassette that directly expresses a secretory form of either Aβ40 or Aβ42 under normal APP regulatory control.

FIGS. 2A-2B depict the results of qRT-PCR quantification of direct Aβ expression in different developmental stages of culture (FIG. 2A) or Aβ and APP expression levels in differentiated neurons (FIG. 2B). FIG. 2A shows that direct expression levels of edit specific Aβ were similar for both edited genotypes and dynamic during early stages of differentiation. Stem cell cultures had intermediate expression, embryoid bodies had significantly less expression and differentiated neurons had the highest, reaching maximal expression by ˜10-20 days after EB dissociation and plating in neural differentiation medium. The relative ratios of Ab expression were ˜1:0.05:5 for the 3 developmental stages. There were no significant differences in expression level comparing edit specific Aβ40 and Aβ42 at any stage (ANOVA, Dunnett's correction). No significant secretory Aβ expression was detected in unedited H9 samples. Data were from 6 independent stem cell cultures, 4 EB stage cultures and 22 individual 10-20 day old differentiated neuronal cultures. FIG. 2B shows that editing did not affect APP expression from unedited alleles. Primer pairs spanning 3 different APP exons were used. The pattern of expression was similar for all 3 genotypes and average relative expression for the primer pairs was 1:0.71:0.5 for H9:Aβ40:Aβ42 and was consistent with expected inactivation of one APP due to editing. Expression of edit specific Aβ was ˜30-fold less than APP expression and was replotted from FIG. 2A for comparison. Data were from 4 independent differentiations of H9 cells and 8-20 differentiations for edited genotypes taken from 10-34-day old cultures. In FIG. 2A expression was measured by qRT-PCR using a forward primer specific to the secretory signal sequence (not present in the human genome) and reverse primer to a sequence common to Aβ40 and Aβ42. In FIG. 2B the forward and reverse primers spanned indicated exons in the APP sequence. Bars are mean normalized expression (MNE) relative to GAPDH (±STD).

FIGS. 3A-3B show that editing did not significantly affect early stage neuronal differentiation. FIG. 3A shows representative images of 10-day old cultures stained with antibodies to DCX (doublecortin, green) to visualize early stage neuronal differentiation, NeuN (red) to visualize more mature neurons and DAPI (blue) to assess total cell number. FIG. 3B shows the quantification of positively stained cells for each marker indicating that there were no genotype specific differences (ANOVA, Dunnett's correction). Bars are the mean (SEM) of 3 biological replicates. Scale bar=30 μm.

FIGS. 4A-B: FIG. 4A shows representative morphological appearance of stem cell cultures, embryoid bodies (28-day old) and 1-day old cells in neuronal differentiation media. Scale bar=500 μm, stem cells; 50 μm, EBs, 10 μm, neurons. FIG. 4B shows immunocytochemical staining of sectioned 7-day old embryoid bodies. Red=anti-Nestin, green=anti-Oct4, Blue=DAPI.

FIG. 5 shows representative Hoffman interference contrast images of unedited H9 parental cells and two independently isolated clones for each edited genotype (Aβ40:#31, #41 and Aβ42:#14, #26) at different culture ages. Isolated cells in 1-day cultures began to cluster together a few days after plating. By ˜10-15 days of differentiation all 3 genotypes formed more recognizable neuronal clusters (NC) which were attached to the culture surface and elaborate neural processes which connected with adjacent NCs. Morphologic appearance of all 3 genotypes was generally similar up to ˜30-40 days of culture. The absolute size of NCs varied across independent differentiations, however, there were no significant differences among the 3 genotypes up to ˜30 days of age (ANOVA, Dunnett correction). After ˜20-30 days, Aβ42 genotypes began to exhibit a granular and darker appearance (especially evident in the Aβ42 clone #26 30-day image) and the somal regions were no longer firmly attached to the culture surface but tethered by their neuronal processes. After 50-60 days, essentially all Aβ42 genotypes exhibited this type of morphology as did many of the Aβ40 cultures at culture times greater than ˜70-90 days. No consistent clone specific differences were observed for edited genotypes. Scale bars=10 mm for 1-day culture and 100 mm for other ages.

FIG. 6 is a graph quantifying Rab4+ staining and accompanying images depicting abnormal AEL structures in Aβ₁₋₄₂ modified HEK cells. Rab4+ objects increased in number. Mean±SD, ANOVA, P<0.01. Specific increase in endosomal marker expression (Rab4, red) in HEK cells edited to express Aβ₁₋₄₂. Blue staining is DAPI (nuclei). Values are expressed as mean red area±SEM for several biological replicates (p <0.01, ANOVA corrected for multiple comparisons).

FIG. 7 is a graph depicting results of a CFSE viability assay of unmodified, Aβ₁₋₄₂ modified, and Aβ₁₋₄₀ modified HEK cells. A non-linear curve was fit to each set of data points and the 95% confidence intervals are indicated with dotted lines. The Aβ₁₋₄₂ edited HEK cells were significantly less viable after ˜4 days in culture.

FIG. 8 is a schematic diagram illustrating a strategy to produce red, green and blue lineage reporters. The constructs can genetically label astrocytes, neurons and microglia with fluorescent reporter genes. The gray shaded box indicates coding exons for each targeted locus.

FIG. 9 shows a set of images illustrating the significantly increased accumulation of aggregated Aβ in 32-day old cultured neurons with edited genotypes compared to unedited H9 cells. Cultures were stained with an Aβ₁₋₄₂ aggregate specific antibody (7A1a, red) and a neuronal marker (Tuj1, green). Image analysis indicates a significant accumulation of Aβ₁₋₄₂ relative to unedited Aβ₁₋₄₀ or H9 neurons. ANOVA, p values corrected for multiple comparisons.

FIGS. 10A-10D show that accumulation of aggregated/oligomeric Aβ was time dependent and more prominent in Aβ42 relative to Aβ40 edited cultures and was associated with pyknotic nuclei, even in unedited H9 samples. FIG. 10A; Maximum intensity Z-projections of NCs fluorescently stained with anti-Tuj1 (neuronal, green) and anti-Aβ7A1a (aggregated/oligomeric Aβ, red) antibodies in 32- or 63-day old cultures. Consistently, the area of 7A1a positive staining was greater in Aβ42 NCs, intermediate in Aβ40 NCs and much lower in unedited H9 cultures. Staining was primarily intracellular and initially appeared as small puncta which were more obvious in areas of lower staining intensity. FIG. 10B: Box and whisker plot of relative 7A1a staining in individual NCs (normalized to Tuj1 staining). The line in the box was the median value, whiskers were the range. Data was from 4 independent differentiations. NCs from Aβ42 cultures had significantly greater accumulation of aggregated/oligomeric Aβ at 32-days (ANOVA, Dunnett correction) relative to H9. Accumulation of Aβ40 in cultures appeared higher than H9 but was not significant at this age. Mean relative accumulation of 7A1a staining ±SEM were: H9=1±0.235, Aβ40=3.77±0.704, Aβ42=6.93±1.63. In 63-day old cultures, both Aβ40 and Aβ42 were significantly different relative to H9. The mean relative areas are: H9=1±0.157, Aβ40=2.34±0.287, Aβ42=3.959±0.337). FIG. 10C: 7A1a staining was present primarily in areas near pyknotic/fragmented DAPI stained nuclei (i.e. small intensely fluorescent structures, arrowheads) and absent from cells with normal nuclei (i.e. large, weak DAPI fluorescence, arrows). Images are from a single optical section of a 32-day old Aβ42 sample (top row) with a magnified view (bottom row) of the indicated rectangular area. FIG. 10D: Association of 7A1a and pyknotic nuclei was not dependent on editing. Left panel shows images of fragmented or intact nuclei from Aβ42 or H9 cultures. Right panel shows spatial distribution of 7A1a fluorescence relative to the center of mass for DAPI staining. Bars are the mean (SEM) area of 7A1a staining in individual concentric circles centered on the DAPI staining. Data is from at least 60 nuclei or pyknotic nuclei from 3 independent differentiations of 32/34 day old cultures. Scale bar in FIG. 10A=10 μm, FIG. 10C=20 μm, FIG. 10D=4 μm.

FIG. 11 shows orthogonal projections of 7A1a staining indicating a primarily intracellular accumulation. A maximum intensity projection of a 32-day old neuronal cluster from an Aβ42 edited culture (main image). XZ (bottom) and YZ (right) projection images of the same image stack are shown on the bottom and right. The red 7A1a positive staining (aggregated/oligomeric Aβ) was primarily near blue (DAPI) stained nuclei or pyknotic nuclei and largely within the limits of green (Tuj1) positive neuronal staining. Scale bar is 10 μm. A 60 optical slice stack (0.07 μm spacing) was initially processed in FIJI to correct for uneven illumination using the CIDRE plugin (Smith, K., et al. (2015)). The image was cropped and a maximum intensity projections and orthogonal projections generated in FIJI using a 30 slices sub stack (2.1 pm of total depth). The final image was assembled in Adobe Photoshop (CS4) and adjusted for brightness and contrast.

FIG. 12 shows the immunocytochemical staining of Aβ in 32-day old cultures using antibody 6E10. There was more Aβ accumulation in the cytoplasm in unedited H9 cells relative to Aβ42 edited cells at this stage. This suggests that this staining may be due to amyloidogenic processing of APP since the edited cells are heterozygous for APP. The brightness and contrast of the red channel has been adjusted the same for both images to show the comparative intensity of staining.

FIG. 13 shows a set of images for 34-day old cultures stained with an antibody to Synapsin 1 (green). Aβ₁₋₄₂ edited cultures had significantly fewer synapses relative to unedited H9 neurons. Student's t test, p=00203. Nuclei are indicated by DAPI staining (blue). Aβ₁₋₄₂ edited neurons also had a decreased number of synapses relative to Aβ_{1-40.}

FIG. 14 shows that Aβ42 edited NCs had fewer synapsin1 stained puncta in 34-day old cultures. Images are maximum intensity projections of 3 adjacent 0.05 μm spaced optical sections stained with anti-synapsin1 (synaptic marker, green) and anti-NeuN (mature neurons, red) antibodies and DAPI (total cells, blue). Synapsin1 positive puncta were counted in individual NCs from 3 different differentiations normalized DAPI and analyzed (ANOVA, Dunnett corrected). The number of synapsin1 puncta was significantly less for Aβ42 cultures. The relative number of puncta ±SEM were: H9=1±0.198, Aβ40=0.618±0.065, Aβ42=0.492±0.081. Data was from 3 independent differentiations. Scale bar=20 μm.

FIG. 15 shows a set of images indicating culture morphology of neuronal clusters at the indicated ages (grayscale, Hoffman DIC) and fluorescent live-dead staining of the same field (calcein AM (live cells, green) and ethidium homodimer dead cells (red). fAβ₁₋₄₀ edited (center panels), and Aβ₁₋₄₂ edited neurons. The images demonstrate that neurons modified to directly express Aβ₁₋₄₂ resulted in age dependent Aβ₁₋₄₂ specific death cell death relative to neurons modified to express Aβ₁₋₄₀ or unedited cells. Representative images (Hoffman DIC, grayscale and fluorescence, color) for each genotype at the indicated ages following neuronal differentiation. Green cells are alive, red cells are dead.

FIG. 16 indicates the percentage of cell death in neuronal clusters for the unedited (H9), Aβ₁₋₄₀ edited (Aβ40), or Aβ₁₋₄₂ edited (Aβ42) neurons at different age cultures assayed as shown in FIG. 15. There was a specific increase in dead (red) cells relative to the other 2 genotypes. ANOVA, p value corrected for multiple comparisons. Quantitative analysis of % dead cells (red) indicating specific ND for Aβ₁₋₄₂ genotype after ˜70 days of culture. ANOVA, p<0.001, Tukey correction ±95% Cl).

FIGS. 17A-17B show that Aβ42 and Aβ40 edited cultures underwent chronic progressive ND. FIG. 17A: Hoffman extended depth-of-field images (left) with a corresponding fluorescent maximum intensity projection (right) at 3 different culture ages. Green fluorescence (calcein-AM) and red fluorescence (ethidium homodimer) were used to estimate live or dead cells. FIG. 17B: Quantitation of relative ratio of dead/live cells. Relative to unedited H9 cultures there were significantly more dead neurons in Aβ42 samples at all three culture ages (ANOVA, Dunnett corrected). Aβ40 samples had significantly more dead neurons but only in cultures older than 60 days. Mean values (±SEM) for 10-day old samples were: H9=1.173±0.289, Aβ40=3.4±0.643, Aβ42=4.49±1.471; for 34-39-day old samples: H9=23.9±3.226, Aβ40=20.79±2.025, Aβ42=35.00±2.974, and for >60-day old samples: H9=16.02±1.612, Aβ40=32.36±3.016, Aβ42=38.46±1.588. Each data point represents an individual NC collected from a total of 8 individual differentiations. The line inside the box is the median and the whiskers are the range. Scale bar=100 μm.

FIGS. 18A-18B show that the number of LAMP1 positive vesicles was affected by editing. FIG. 18A: Fluorescence maximum intensity projections of 2 adjacent optical sections stained with anti-LAMP1 antibody (green) or DAPI (blue) at 2 different culture times. FIG. 18B: The relative number of LAMP1 positive puncta (normalized to DAPI) in individual NCs was greater in 38-day old Aβ42 samples relative to H9. In 62-day cultures both Aβ42 and Aβ40 (not significant) samples had fewer LAMP1 objects relative to H9 (ANOVA, Dunnett corrected). Data are from 3 independent differentiations of 38-day cultures and 2 independent differentiations of 62-day cultures. Mean values (±SEM) for 38-day samples were: H9=1±0.139, Aβ40=0.84±0.059, Aβ42=2.064±0.142 and for 62-day samples: H9=1±0.205, Aβ40=0.553±0.106, Aβ42=0.453±0.101. The line inside the box is the median and the whiskers are the range. Scale bar =10 μm.

FIGS. 19A-19B show that the number of other endolysosomal vesicles was affected by editing. Rab5 positive objects in NCs was greater in 38- to 42-day old Aβ42 samples. At a later culture age (62 days) both Aβ40 and Aβ42 samples had fewer LAMP1 objects. FIG. 19A: maximum intensity projections of 2 adjacent optical sections (1 pm spacing) stained with anti-Rab5 antibody (early endosome marker, red) and DAPI (blue). FIG. 19B: The relative number of Rab5 puncta (normalized to DAPI) was greater in 38- to 42-day old cultures for Aβ42 edited samples and less for both Aβ42 and Aβ40 edited samples in 63-day cultures (ANOVA, Dunnett corrected). Data is from individual NCs from 3 independent differentiations for 38- to 42-day old cultures and 2 independent differentiations for 63-day cultures. Mean values (±SEM) for 38- to 42-day old samples are: H9=1±0.1448, Aβ40=2.39±0.2767, Aβ42=4.80±1.333 and for 63-day samples are: H9=1±0.1584, Aβ40=0.586±0.071, Aβ42=0.4198±0.0341.

FIGS. 20A-20B shows that older Aβ42 cultures demonstrated apparent distribution of phospho-tau in cell soma compared to H9 cultures where it was localized in neurites. FIG. 20A: Fluorescence images from 3 representative fields for each genotype taken from a 62-day old culture stained with anti-phospho-tau antibody (green) and DAPI (blue). This apparent difference was likely due to a significant decrease in neurites on dead or dying cells present in Aβ42 cultures rather than a redistribution of signal. The area of phospho-tau staining (normalized to DAPI) was not significantly different between H9 and Aβ42 samples (p=0.9078, N>15, t test). Scale bar=20 μm. FIG. 20B: RNA-Seq analysis indicates no significant difference in relative MAPT expression (coding for tau) among the genotypes (ANOVA, Dunnett corrected). Data points are from independent RNA-Seq samples (±SEM).

FIG. 21 shows that the relative number of Rab3A and LC3B puncta were more variable in individual samples of all genotypes and both decreased primarily in older cultures. Individual NCs from 2 independent differentiations were stained with either anti-Rab3A (synaptic vesicle associated marker) or LC3B (autophagosome marker) antibody. There was a significant decrease in LC3B objects in Aβ40 samples at 43 days and a decrease in both Aβ40 and Aβ42, as well as LC3B puncta, in 63-day cultures (ANOVA, Dunnett corrected). Bars are mean ±SEM, N=5-20. Scale bar=20 μm.

FIG. 22 shows the expression levels of selected vesicular genes. qRT-PCR analysis (Syber green) of selected vesical associated marker genes. Mean normalized expression values relative to GAPDH. Individual points are samples taken from independent differentiations. There were no significant differences in the level of expression for these genes among the 3 genotypes (ANOVA, Dunnett corrected). Cultures were 34-42 days old.

FIGS. 23A-B: FIG. 23A shows the heat map of differentially expressed genes in 34-day old Aβ₁₋₄₂ cultures relative to H9 and Aβ₁₋₄₀ following RNAseq analysis. Hierarchical clustering indicates that H9 and Aβ₁₋₄₀ were similar to each other while Aβ₁₋₄₂ appeared different. FIG. 23B is the ingenuity pathway analysis showing the highest and lowest scoring function/disease pathways. Both are of potential relevance to Alzheimer's disease. Expression levels are indicated with color and the connecting spokes indicate the predicted direction of change. The top IPA Canonical Pathway is neuroprotective Role of Thop1 in Alzheimer's (Ab42 vs H9). Genes: MME (Neprylisin) and SERPINA3 (ACT), p=9.95 E-03, Overlap=5% (2/40).

FIGS. 24A-24C show differentially expressed genes in 34-day old cultures. FIG. 24A: Cluster analysis of differentially expressed genes (Pearson dissimilarity metric). Aβ42 samples cluster together while Aβ40 and H9 samples overlap. FIG. 24B: Heat map of significant DEGs from RNA-Seq analysis of Aβ42 vs H9 comparison and Aβ40 vs H9 comparison. Up (UP) regulated genes (red) and down (DN) regulated genes (green) were sorted by the magnitude of the indicated fold change (FC) values for the Aβ42 vs H9 comparison. There was a general correspondence in the directional FC values with a relatively larger FC in the Aβ42 samples. FIG. 24C: Pearson correlation confirmed significant co-variation of the log2 ratios of all genes (top, significant DEGS in color) as well as the FC values for significant UP and DN regulated genes (bottom).

FIG. 25 shows that DEGs are potentially related to Alzheimer's relevant pathways and functions. IPA pathway analysis of DEGs in the Aβ42 vs H9 comparison identified “decreased memory” (z=−2.213) and “increased neuronal cell death” (z=1.658) as the lowest and highest scoring functional pathways. Individual genes are shown as graphic symbols representing molecule type and color coded by FC values (red=UP, green=DN). The most relevant IPA disease related canonical pathway was “Neuroprotective role of THOP1 in Alzheimer's disease” (p=9.95 E-03, overlap=2 of 40 total genes in this pathway). The pathway genes were MME (aka NEP, neprilysin) an Aβ degrading metalloproteinase and SERPINA3 (aka ACT, alpha-1 antitrypsin) a protease inhibitor found in AD plaques (circled in blue). MME is not directly included in the IPA “decreased memory” function but can potentially be indirectly related through its relationship to GRP.

FIG. 26 shows the genomic PCR to identify the correct HR.

FIG. 27 shows the Surveyor Assay for TALEN pairs.

FIGS. 28A-28B show cell type specific and AD related gene expression.

FIG. 29 shows the clustered heat map showing relative sample level expression for IPA (Ingenuity Pathway Analysis) identified “Decreased Memory” and “Increased Neuronal Death” related genes.

### DETAILED DESCRIPTION

Recombinant stem cells, in vitro model systems using said recombinant stem cells, and methods of screening potentially therapeutic agents and/or treatment regimens are provided herein. According to the embodiments described herein, the recombinant stem cells may be differentiated into a plurality of cell types for subsequent use in in vitro model systems and for systems and methods designed to assess the effects of potentially therapeutic agents and/or treatment regimens.

### Recombinant Cells

In certain embodiments, an expression cassette for expressing a modified amyloid β peptide in a cell is provided. The expression cassette may be delivered to a stem cell using any suitable manner including, but not limited to, a vector (e.g., a recombinant viral vector or plasmid) or a gene editing method (e.g., TALEN-based genome editing, CRISPR/Cas9 editing, zinc finger nuclease (ZFN)-based editing). Stem cells that may be used in accordance with the embodiments described herein include, but are not limited to, embryonic stem cells, somatic stem cells, induced pluripotent stem cells (iPS), or any other cell line that can give rise to a cell found in the nervous system (e.g., neurons or glia). Alternatively, the embodiments described herein may relate to modification of cell lines that comprise already-differentiated neurons or glia.

In some embodiments, the expression cassette described herein includes a modified amyloid beta precursor protein (APP) gene. Examples of the modified APP gene and how the modified APP gene can be used to modify a cell are described below.

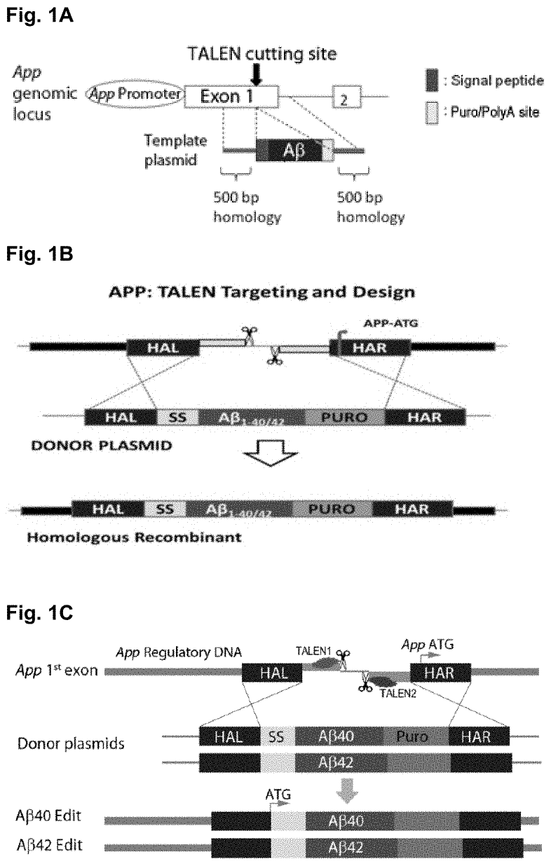

In a first embodiment, a stem cell is modified to express an amyloid β peptide (Aβ) that is encoded by a modified APP gene. In this embodiment, the resulting recombinant cell is modified to include a modified amyloid β peptide (Aβ) coding sequence. In such a case, the normal APP gene is modified such that a secretory form of Aβ₁₋₄₂ is produced in place of APP. In this embodiment, the cell does not depend on amyloidogenic processing of APP to generate proteotoxic Aβ₁₋₄₂. In this embodiment, the Aβ₁₋₄₂ peptide is under the control of normal APP regulatory DNA and the peptide product is routed through the normal secretory pathway. In this embodiment, modification may be optionally achieved using transcription activator-like effector nuclease (TALEN)-based genome editing (FIGS. 1A-C). For example, H9 cells—a widely used human embryonic stem cell (ESC) line—can be modified by targeting a site within the first non-coding exon of the APP gene. Homologous recombination is used to simultaneously eliminate APP expression and replace it with a coding sequence for a secretory form of Aβ₁₋₄₂. Cells that have been modified as described in this embodiment can optionally be used to model Aβ₁₋₄₂-dependent neurodegeneration following a selected differentiation protocol. Cells from this embodiment can be optionally differentiated into a plurality of cell types including, for example, cholinergic motor neurons, astrocytes or microglia. In this embodiment, modification may be optionally achieved using the CRISPR/Cas9 editing method, zinc finger nuclease (ZFN)-based editing, lentiviral systems, or any suitable method using any suitable stem cell line.

In a second embodiment, a stem cell is modified to directly express a secretory form of Aβ₁₋₄₀ (rather than Aβ₁₋₄₂ as in the first embodiment). Aβ₁₋₄₀ is the major product of APP amyloidogenic processing, but is much less toxic than Aβ₁₋₄₂ even though it is produced at higher levels in AD. In this embodiment, modification is optionally achieved using TALEN-based genome editing. For example, H9 cells can be modified by targeting a site within the first non-coding exon of the APP gene and using homologous recombination to simultaneously eliminate APP expression and replace it with a coding sequence for a secretory form of Aβ₁₋₄₀. (FIGS. 1A-C). Cells that have been modified as described in this embodiment can optionally be used as modified control lines in model systems examining Aβ₁₋₄₂-dependent neurodegeneration following a selected differentiation protocol. Cells from this embodiment can be optionally differentiated into a plurality of cell types including, for example, cholinergic motor neurons, CNS non-limb innervating ChAT+neurons, or astrocytes. In this embodiment, modification may be optionally achieved using the CRISPR/Cas9 editing method, ZFN-based editing, lentiviral systems, or any suitable method using any suitable stem cell line.

According to the embodiments described herein, modified or recombinant stem cells may be used to generate a model of Aβ₁₋₄₂-dependent ND which may in turn be optionally used as an in vitro AD model, a screening platform for potential therapeutic agents, and/or a screening platform for a plurality of treatment paradigms. The rate of Aβ₁₋₄₂ production, is not the only factor resulting in accumulation. Aβ₁₋₄₂ removal is also important. Many potential Aβ₁₋₄₂ removal mechanisms have been discovered, such as specific protease digestion (i.e. nepriylisin, insulin degrading enzyme, etc.) or other important cellular catabolic pathways such as autophagy or proteosomal processing. None, however, have been completely evaluated in the context of progressive ND, primarily because of the absence of this phenotype in mouse models. Additionally, it remains unknown which specific cell types participate in Aβ₁₋₄₂ catabolism. Neurons, astrocytes and microglial cells have been suggested. Thus, subjecting the modified cells described herein to differentiation protocols that favor these cell types will provide useful in vitro models and screening tools.

AD is a very complex disease and a host of non-Aβ₁₋₄₂ genetic and environmental factors influence risk of contracting the disease or rate of progression. APOE allele type is one such genetic factor. APOE allele type is the primary genetic risk factor for developing AD. APOE codes for a 299 amino acid lipoprotein. People with the rare APOE2 allele are less likely to develop AD. In contrast, individuals with even a single APOE4 allele have a much higher risk, even reaching semi-dominance in APOE4/E4 homozygotes (Yu & Hardy 2014). The cellular and biochemical basis for these strong genetic associations affects not only AD but also affects the development of other diseases and conditions, thereby conferring a broader applicability beyond AD. For example, the association affects cardiovascular diseases such as stroke, cerebral amyloid angiopathy and coronary heart disease (Villeneuve et al. 2014). Thus, in certain embodiments, the stem cell platform described herein may be used in applications beyond AD to study the etiology of and to screen for treatments for any disease or condition related to the genetic association with APOE. APOE is expressed in many tissues with highest levels in liver followed by the brain. In the CNS, APOE levels are highest in astrocytes. Neurons express APOE at lower levels but induce higher expression in response to neuronal injury or stress (Mahley et al. 2012).

In another embodiment, stem cells from the embodiments described above may be additionally modified to alter APOE genotype. For example, APOE genotype may be optionally modified from APOE3/E4 as occurs in unmodified parental H9 cells to APOE2/E2, APOE3/E3, or APOE4/E4. As one example, these optional modifications allow direct testing of the cellular specificity of APOE genotype in modulating Aβ₁₋₄₂-dependent ND. Combining APOE genotype with cellular context may be used to generate important tools useful in developing future AD therapies. These modifications may be optionally achieved using CRISPR/Cas9 editing, TALEN-based editing, ZFN-based editing, lentiviral systems, or any other suitable modification technique.

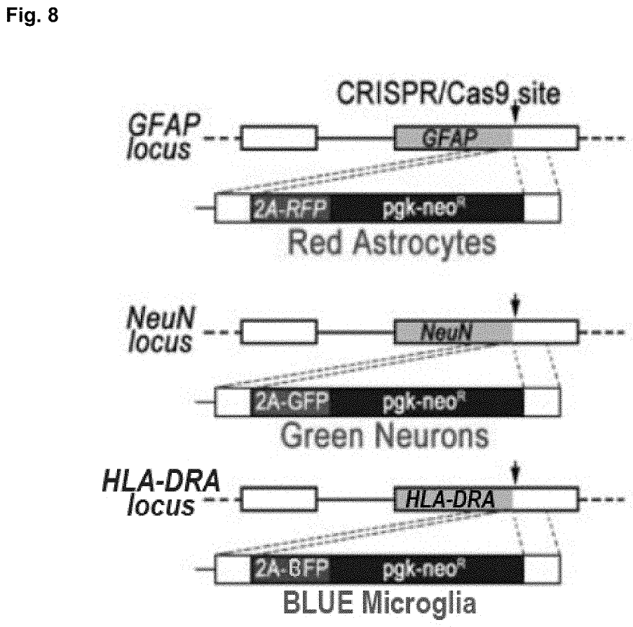

In another embodiment, the modified or recombinant cells described herein may display a marker. The marker may be included in the same expression cassette that includes a modified APP gene, or it may be expressed separately. Incorporating markers may be achieved using CRISPR/Cas9 editing, TALEN-based editing or any other suitable technique. In some embodiments, the marker may be for cell type. The cell type marker may be, for example, a neuronal marker (e.g., neuron specific enolase (NSE), neuronal nuclei (NeuN), microtubule-associated protein 2 (MAP-2), βIII Tubulin, doublecortin (DCX), c-fos, choline acetyltransferase (ChAT), tyrosine hydroxylase (TH), polysialic acid-neural cell adhesion molecule (PSA-NCAM), Neurogenic Differentiation 1 (NeuroD1 or Beta2), tau, calbindin-D29k, calretinin, neurofilament protein (NFP), synaptophysin, PSD95, Neurofilaments, TBR1, stathmin 1). In other embodiments, the cell type marker may be a glial marker (e.g., vimentin, Pax6, HES1, HES5, GFAP, EAAT1/GLAST, BLBP, TN-C, N-cadherin, Nestin, SOX2, 2′, 3′-cyclic nucleotide 3′-phosphodiesterase (CNPase)). In certain aspects, the glial marker may be an astrocyte marker (e.g., glial fibrillary acidic protein (GFAP), S100β, GLAST/EAAT1, EAAT2/GLT-1, glutamine synthetase, ALDH1L1). In other embodiments, the cell type marker may be a microglial marker (e.g., BCL2 Related Protein A1(BCL2A1), C-C Motif Chemokine Ligand 2 (CCL2), C-C Motif Chemokine Ligand 4 (CCL4), CD14 Molecule (CD14), CD83 Molecule (CD83), Colony Stimulating Factor 1 Receptor (CSF1R), G Protein-Coupled Receptor 183 (GPR183), Major Histocompatibility Complex, Class II, DR Alpha (HLA-DRA), Lysosomal Protein Transmembrane 5 (LAPTM5)). In another embodiment, the modified cells described herein may display a marker that is a fluorescent marker that acts as a reporter. The fluorescent marker or reporter may be GFP, RFP, CFP, YFP, mCherry, AcGFP1, DsRed-Monomer, tdTomato, DsRed, DsRed-Express, E2-Crimson, or any other suitable fluorescent protein. A schematic illustrating one optional labeling combination, wherein red and green lineage reporters are generated using CRISPR/Cas9 editing is depicted in FIG. 8.

In some embodiments, an expression cassette that includes a modified APP gene as described herein also includes an inducible promoter system to allow the modified APP gene to be turned on at different times or at a preselected time. Examples of inducible promoters that can be used in accordance with the embodiments described herein include, but are not limited to, T7 bacteriophage system, trc (hybrid) E. coli system, p_{L} (90 ) system, araBAD system, tetracycline (tet) inducible system (or derivative system), Cre-loxP system, Cre-ERT system, Mx1-inducible systems, galactose inducible system, or any other inducible system known in the art.

As described in the embodiments above, an expression cassette that expresses a modified amyloid β peptide (Aβ) (e.g., Aβ₁₋₄₂) can be introduced to a host cell e.g., a stem cell) to target and replace one allele of the cell's existing APP gene. Alternatively, the expression cassette can be delivered to a different site or locus to express the modified amyloid β peptide that is independent from the cell's APP gene. For example, the cassette may be targeted to the AAVS1 site in chromosome 19 using a gene editing method. Other target sites known in the art may also be used.

In summary, modified cells described herein may be used not only to directly express a secretory form of Aβ₁₋₄₂ or Aβ₃₋₄₀, but may also be used to modify disease-associated genotypes (such as APOE), display cell type or lineage markers that may optionally be fluorescent, or any combination of the foregoing.

In vitro models

One specific advantage of stem cells is their ability to differentiate into many different cell types. It is possible to induce stem cells to differentiate under conditions favoring a desired cell type by using specific protocols. Isolated modified stem cells described herein may be subjected to different differentiation protocols designed to favor differentiation into particular cell types. Products of these differentiation protocols may then optionally be co-cultured to facilitate cell type specific analyses of ND or other AD relevant phenotypes such as synaptic loss, accumulation of Aβ₁₋₄₂ or changes in gene expression patterns that may underlie the synaptic loss, Aβ₁₋₄₂ aggregation or neurodegeneration phenotypes.

In one embodiment, an in vitro model is generated by subjecting a population of modified (or recombinant) stem cells described herein to a differentiation protocol. Any suitable differentiation protocol may be used to produce a desired cell type, including but not limited to, brain cells, cardiovascular cells, or any other type of cell related to APOE-related conditions. In one embodiment, the differentiation protocol is designed to favor production of cholinergic motor neurons. At least two such differentiation protocols have been described previously (see, e.g., Amoroso et al. 2013; Maury et al. 2014). These two protocols will be referred to the first (slower) protocol and second (faster) protocol, respectively. Both protocols produced differentiated cultures with between thirty and forty percent cholinergic neurons (ChAT+/Tuj+). However, there are significant differences in the time to reach full neural differentiation, well-to-well variance in cellular morphology, and the mixture of cell types generated by each method. The first (slower) protocol takes around twenty-four days to generate embryoid bodies (EBs) while the second (faster) protocol takes around fourteen days. When EBs are dissociated and plated, neurons (NeuN+cells) initially appear as early as one to two days in the slower protocol but only after more than three days in the faster protocol. The slower method has less well-to-well variability in neuronal morphology. In another embodiment, the differentiation protocol is designed to obtain CNS non-limb innervating ChAT positive neurons (Wicklund et al. 2010). CNS non-limb innervating ChAT+neurons are sensitive to early ND in AD and thus are a useful cell type for studying the disease. The differentiation protocols described in the above embodiments are characterized primarily by the particular mixture and timing of addition of differentiation factors. They are also dependent on dissociating EBs and culturing on a flat tissue culture surface. Another embodiment is to culture cells in a 3D format or matrix which may reflect better the normal development of neurons. This embodiment may have the favorable property of decreasing the time required to observe phenotypic changes in the modified cells with respect to AD relevant phenotypes. This may be a result of increasing the effective concentration of the proteotoxic Aβ peptide by restricting their diffusion when the cells are confined in a 3D or matrix format.

In one embodiment, an in vitro model is generated from a population of modified (or recombinant) stem cells (e.g., modified embryonic stem cells), the population of modified stem cells having a modified APP gene, wherein the modified APP gene encodes a secretory peptide. In one embodiment, the secretory peptide is Aβ₁₋₄₂. In another embodiment, the modified APP gene encodes the secretory peptide Aβ₁₋₄₀. In another embodiment, a population of modified stem cells have been further modified such that their APOE genotype is APOE2/E2. In another embodiment, a population of modified stem cells that express the secretory peptide Aβ₁₋₄₂ have been further modified such that their APOE genotype is APOE2/E2. In another embodiment, a population of modified stem cells that express the secretory peptide Aβ₁₋₄₀ have been further modified such that their APOE genotype is APOE2/E2. In one embodiment, a population of modified stem cells have been further modified such that their APOE genotype is APOE3/E3. In another embodiment, a population of modified stem cells that express the secretory peptide Aβ₁₋₄₂ have been further modified such that their APOE genotype is APOE3/E3. In another embodiment, a population of modified stem cells that express the secretory peptide Aβ₁₋₄₀ have been further modified such that their APOE genotype is APOE3/E3. In another embodiment, a population of modified stem cells have been further modified such that their APOE genotype is APOE4/E4. In another embodiment, a population of modified stem cells that express the secretory peptide Aβ₁₋₄₂ have been further modified such that their APOE genotype is APOE4/E4. In another embodiment, a population of modified stem cells that express the secretory peptide Aβ₁₋₄₀ have been further modified such that their APOE genotype is APOE4/E4.

In one embodiment, an in vitro model is generated from a population of modified (or recombinant) stem cells having a modified APP gene, wherein the modified APP gene encodes a secretory peptide and the secretory peptide is Aβ₁₋₄₂. In another embodiment, the modified APP gene encodes the secretory peptide Aβ₁₋₄₀. In another embodiment, the modified stem cells described herein have undergone a differentiation protocol that favors generating cholinergic neurons (ChAT+/Tuj+). In another embodiment, an in vitro model is generated from a population of modified stem cells encoding the secretory peptide is Aβ₁₋₄₂ or Aβ₁₋₄₀ and the modified stem cells have undergone a differentiation protocol that favors generating CNS non-limb innervating ChAT+neurons. In another embodiment, an in vitro model is generated from a population of modified stem cells encoding the secretory peptide is Aβ₁₋₄₂ or Aβ₁₋₄₀ and the modified stem cells have undergone a differentiation protocol that favors generating astrocytes. In another embodiment, an in vitro model is generated from a population of modified stem cells encoding the secretory peptide is Aβ₁₋₄₂ or Aβ₁₋₄₀ and the modified stem cells have undergone a differentiation protocol that favors generating microglia. In another embodiment, an in vitro model is generated from a population of modified stem cells encoding the secretory peptide is Aβ₁₋₄₂ or Aβ₁₋₄₀ and the modified stem cells have undergone a differentiation protocol similar to the above embodiments and the cells will be differentiated in a 3D or matrix format.

In one embodiment, an in vitro model is generated from a population of modified (or recombinant) stem cells having a modified APP gene, wherein the modified APP gene encodes a secretory peptide and the secretory peptide is Aβ₁₋₄₂. In another embodiment, the modified APP gene encodes the secretory peptide Aβ₁₋₄₀. In another embodiment, the modified stem cells have at least one marker or reporter. In another embodiment, the modified stem cells have a plurality of markers or reporters. In some embodiments, the marker is a fluorescent marker or reporter. In some embodiments, the fluorescent marker or reporter is GFP. In some embodiments, the fluorescent marker or reporter is RFP, CFP, YFP, mCherry, AcGFP1, DsRed-Monomer, tdTomato, DsRed, DsRed-Express, E2-Crimson, or any other suitable fluorescent protein. In some embodiments, the marker is a cell type marker. In some embodiments, the marker is a neuronal marker. In some embodiments, the marker is an astrocyte marker. In some embodiments, the marker is a glial marker. In some embodiments, the marker is a cell type marker that is also fluorescent.

The in vitro models derived from the recombinant stem cells described herein are comprised of a population of recombinant neuronal cells made up of differentiated human cells of a desired cell type. The population of differentiated human cells include a modified amyloid β peptide (Aβ) coding sequence that causes the cell to express an Aβ encoded by the Aβ coding sequence. In certain embodiments, the Aβ coding sequence is an Amyloid Beta₁₋₄₀ (Aβ₁₋₄₀) coding sequence or an Amyloid Beta₁₋₄₂ (Aβ₁₋₄₂) coding sequence. In certain aspects, the desired cell type is a motor neuron, a cholinergic motor neuron, or a CNS non-limb innervating neuron.

In one embodiment, an in vitro model is generated by co-culturing modified stem cells having a modified APP gene with cells from another source. In one embodiment, an in vitro model is generated by co-culturing modified stem cells having a modified APP gene that have been subjected to a differentiation protocol with cells from another source. In one embodiment, the population of modified stem cells have a modified APP gene, wherein the modified APP gene encodes a secretory peptide and the secretory peptide is Aβ₁₋₄₂. In another embodiment, the modified APP gene encodes the secretory peptide Aβ₁₋₄₀. In another embodiment, the differentiation protocol favors generating cholinergic neurons (ChAT+/Tuj+). In one embodiment, the differentiation protocol that favors generating CNS non-limb innervating ChAT+neurons. In one embodiment, the modified stem cells have undergone a differentiation protocol that favors generating astrocytes. In one embodiment, the modified stem cells have undergone a differentiation protocol that favors generating microglia. In one embodiment, modified stem cells that have undergone a differentiation protocol that favors generating cholinergic neurons (ChAT+/Tuj+) are co-cultured with modified stem cells that have undergone a differentiation protocol that favors generating astrocytes. In one embodiment, modified stem cells that have undergone a differentiation protocol that favors generating CNS non-limb innervating ChAT+neurons are co-cultured with modified stem cells that have undergone a differentiation protocol that favors generating astrocytes. In another embodiment, modified stem cells that have at least one marker are co-cultured with cells from another source. In one embodiment, the co-cultured modified stem cells have a neuronal marker. In one embodiment, the co-cultured modified stem cells have an astrocyte marker. In other embodiments, the co-cultured modified stem cells have a glial marker. In other embodiments, the co-cultured modified stem cells have more than one marker. In other embodiments, the co-cultured modified stem cells have at least one fluorescent marker. In other embodiments, the co-cultured modified stem cells have more than one fluorescent marker.

Methods for screening potential therapeutic agents and/or treatments

Modified stem cells described herein may serve as a screening platform for potentially therapeutic agents and/or treatments for neurodegeneration. As such, methods for screening agents for treatment of a neurodegenerative condition are provided herein. In one embodiment, a method for screening agents for a neurodegenerative condition includes a stem of measuring an initial level of Aβ₁₋₄₂ produced by a population of recombinant cells, wherein the population of cells comprises a modified APP gene which comprises an amyloid β peptide (Aβ) coding sequence, and wherein the recombinant stem cell directly expresses an Aβ encoded by the Aβ coding sequence. Measurement of Aβ₁₋₄₂ can be accomplished by any suitable detection method. AD neurodegeneration is characterized by neuronal death and preceded by a loss of synapses. As such, additional screening methods provided in the embodiments described herein will measure synaptic loss and the extent or rate of neuronal death. Measurement of synaptic numbers or cell death can be accomplished by any suitable detection method known in the art.

After measuring the initial level of Aβ₁₋₄₂, the population of cells are contacted with a candidate therapeutic agent that targets Aβ₁₋₄₂ (e.g., a candidate aptamer, antibody, oligonucleotide, RNAi molecule, peptide, protein, small molecule, or any other molecule capable or putatively capable of reducing Aβ₁₋₄₂, levels). The agent may target Aβ₁₋₄₂, directly or may target any molecule upstream or downstream of Aβ₁₋₄₂, which, when antagonized or agonized, will result in a reduction of Aβ₁₋₄₂, levels. The number of synapses as well as the extent or rate of neuronal cell death may also be measured.

After contacting the population of cells with the candidate therapeutic agent, a second level of Aβ₁₋₄₂, produced by the population of cells is measured. If the second level of Aβ₁₋₄₂ is lower than the initial level, the candidate therapeutic agent is identified as a candidate for treating the neurodegenerative condition. The number of synapses as well as the extent or rate of neuronal cell death may also be measured.

In one embodiment, the agent is a drug. In another embodiment, the agent is a salt. In another embodiment, the agent is an antibody. In another embodiment, the agent is an enzyme. In another embodiment, the agent is a protein. In another embodiment, the agent is a peptide. In another embodiment, the agent is a cell. In another embodiment, the agent is a modified cell. In another embodiment, the agent is plant-based. In another embodiment, the agent is an antioxidant. In one embodiment, the treatment includes stem cell therapy. In another embodiment, the treatment includes an agent and stem cell therapy. In another embodiment, the treatment includes a plurality of agents. In another embodiment, the treatment includes a plurality of agents and stem cell therapy.

Many genes, molecules, cell types and pathways have been implicated in AD. This wealth of information comes from numerous patient observation or investigation of a variety of experimental AD models but has not yet been developed into effective treatments. The three interrelated factors which may account for the clinical failures are disease complexity; limited experimental accessibility of patient material; and phenotypic deficiencies or artifacts in all available AD models (Ashe and Zahs, 2010; Bales, 2012; Sasaguri et al., 2017). As disclosed herein, the complexity of amyloidogenic APP processing is reduced by directly expressing genetic constructs coding for secretory forms of either Aβ40 or Aβ42. Other AD models have also used a direct expression approach (Abramowski et al., 2012; Iijima-Ando and Iijima, 2009; LaFerla et al., 1995; Lewis et al., 2001; McGowan et al., 2005), but the disclosed model differs in several important ways. First, genomic editing of the App gene rather than Aβ transgene over-expression is used. Direct Aβ expression is thus under control of the normal App regulatory DNA and should be expressed at normal levels when and where App is normally expressed. Over-expression artifacts are not likely to explain our phenotypic results. Some of the most widely used mouse transgenic AD-models have phenotypes that seem related to this common problem (Saito et al., 2014; Sasaguri et al., 2017). Second, edit specific phenotypes are compared to unedited isogenic parental cells. Third, human neurons which likely better capture interactions and processes related to AD are used.

AD has many complex genetic associations. The difference in edited genotypes compared to unedited parental cells in the disclosed model is homologous recombination of the editing cassette in the first intron of App which results in App heterozygosity. App heterozygosity, however, is unlikely to directly explain the phenotypic results since independently generated Aβ40 or Aβ42 lines are both heterozygous, yet they display significant differences in phenotypic initiation and rate of progression. Early developmental functions of APP have been described (Freude et al., 2011), but both edited genotypes developed and differentiated into neurons essentially identical to unedited parental cells. Additionally, APP mRNA levels in edited lines, while consistent with heterozygosity, maintained the same relative distribution of several different qRT-PCR amplicons compared to unedited samples suggesting that editing did not significantly affect the pattern of APP mRNA processing of the unedited allele. Homozygous deletion of App in mice also has limited phenotypic consequence (Muller and Zheng, 2013; Zheng and Koo, 2006) while heterozygous deletions were indistinguishable from wild-type littermates (Zheng et al., 1995). Finally, parallel observations were made on at least two independently isolated clones that for each edited genotype and phenotypic the results were similar, suggesting that they were not due to any off target genetic changes. Thus, all edit specific phenotypes can be attributed to direct expression of either Aβ40 or Aβ42.

An overwhelming body of evidence has been formalized into the dominant “Amyloid Hypothesis” of AD which suggests that structurally imprecise Aβ oligomers are responsible for initiating a cascade of downstream progressive pathology terminating in ND (Selkoe and Hardy, 2016). Nevertheless, unresolved conflicts regarding the pathogenic significance of Aβ to AD remain (Benilova et al., 2012; Musiek and Holtzman, 2015; Selkoe and Hardy, 2016). Oligomer formation is complex and incompletely defined, especially with respect to demonstrations of neuronal toxicity (Benilova et al., 2012; Teplow, 2013; Walsh et al., 2000). The mechanistic details linking Aβ oligomers to downstream molecular and cellular processes are also not entirely clear. Part of the reason for these conflicts may be related to the absence of AD human culture models (Arber et al., 2017; Mungenast et al., 2016) or amyloidogenic rodent models (Sasaguri et al., 2017) that have progressive ND phenotypes. One finding of this disclosure is that direct Aβ expression appears sufficient to produce chronic progressive ND of cultured human neurons. Oligomeric/aggregated Aβ accumulates intracellularly in differentiated neurons and precedes the appearance of several other AD-like phenotypes. Amyloidogenic Aβ production is thus not strictly necessary for AD-like phenotypic development. Minimizing amyloidogenic APP processing, however, raises issues of direct relevance to AD.

Chronic progressive ND

Stem cell maintenance, embryoid body formation and neurogenesis were not affected by editing. Only later stage differentiated neurons were subject to edit specific cell death. The culture age when reduced neuronal viability was first detected was significantly earlier for Aβ42 and progressed at a faster rate compared to Aβ40. Live neurons were not observed in edited cultures older than ˜120 days while parental cultures appeared normal and remained healthy up to 266 days. Taken together these results indicate that direct expression of either Aβ40 or Aβ42 has little effect on undifferentiated stem cells or neuronal early development or neuronal differentiation but rather initiates a month's long process of chronic progressive neuronal death in the absence of high level amyloidogenic APP processing.

In contrast to the results demonstrated in the working examples, similar invertebrate and mammalian models (Abramowski et al., 2012; Iijima-Ando and Iijima, 2009; LaFerla et al., 1995; Lewis et al., 2001; McGowan et al., 2005) all exhibit prominent ND for Aβ42 but not Aβ40 direct expression. Perhaps some human specific factor could explain this difference. One possibility is that formation of toxic Aβ oligomer/aggregate structures differs in the specific genetic context of the model. For example, oligomer formation of different mixtures of Aβ40 and Aβ42 is dependent on their relative concentrations and further suggested to require an Aβ42 “seed” (Kim et al., 2007; McGowan et al., 2005). Drosophila do not contain any Aβ homologous sequence in the Appl gene (the fly APP homolog) thus cannot produce Aβ42 via amyloidogenic processing. The absence of Aβ40 ND may reflect the absence of Aβ42. Mammalian direct expression models, however, all produce rodent APP from an endogenous gene which is proteolytically processed into Aβ40 and Aβ42 via the amyloidogenic pathway. The murine Aβ sequences however differ by 3 amino acids relative to the directly expressed human Aβ transgenes. Perhaps unknown mixtures of rodent/human Aβ interfere with formation of neurotoxic oligomers and prevent Aβ40 dependent ND. As shown in the working examples, both directly expressed and amyloidogenic Aβ would have an identical human sequence. An Aβ42 dependent seeding mechanism could thus explain Aβ40 toxicity since small amounts of amyloidogenic Aβ oligomer was routinely observed in unedited cells and this also likely occurs in edited cells which all contain an unedited App allele. It is also possible that an Aβ42 seeding mechanism could relate to the slower phenotypic elaboration Aβ40 edited neurons relative to Aβ42 since both direct expression and amyloidogenesis could provide an appropriate seed.

Intracellular Aβ accumulation

Direct expression of Aβ40 or Aβ42 in human neurons resulted in an intracellular accumulation of oligomeric/aggregated peptides detected by 7A1a antibody staining, even though both editing constructs included a normal secretory pathway routing sequence. Coupled with the inability to detect secreted Aβ using ELISA assay, this indicates that direct expression preferentially retains or rapidly reinternalizes Aβ. This differs from other human AD culture models that produce Aβ via amyloidogenic APP processing. High levels of Aβ accumulate extracellularly in culture media suggesting that Aβ is mostly secreted (Arber et al., 2017; Mungenast et al., 2016). Using either immunocytochemistry or ELISA assay, only relatively low levels of intracellular Aβ accumulate in these other culture models (Kim et al., 2015; Kondo et al., 2013; Raja et al., 2016; Shi et al., 2012). Extracellular plaque-like Aβ aggregates have also been demonstrated in amyloidogenic culture models that use 3D culture support or organoid differentiation. These approaches likely restrict diffusion of secreted Aβ allowing extracellular aggregates to form (Kim et al., 2015; Raja et al., 2016). Intracellular Aβ protein was unable to be detected using ELISA assay of cell extracts, a finding also observed in some other amyloidogenic human culture models (Israel et al., 2012; Muratore et al., 2017). Low levels of edit specific transcripts measured in the disclosed cells suggest that Aβ protein levels may also be quite low and possibly explain this negative result.

Intracellular Aβ oligomer accumulation correlates best with the rate of ND in the cultures. Amyloidogenic APP processing occurs at the plasma membrane, where Aβ is released into extracellular spaces where it aggregates into plaques that maintain a dynamic equilibrium serving as an extracellular oligomer source (Haass and Selkoe, 2007). Intracellular vesicle compartments also produce amyloidogenic Aβ which could be preferentially retained (Haass and Selkoe, 2007). Secretion and reuptake of Aβ has also been demonstrated in cultured neurons or early stage AD patient samples (Hu et al., 2009). In other direct expression AD models Aβ42 also appears to be preferentially retained or endocytosed by neurons relative to Aβ40 (Abramowski et al., 2012; Ling et al., 2014). Vesicular Aβ42 in Drosophila neurons following direct Aβ expression is primarily oligomeric and accumulates in vesicles with a range of endocytic, autophagic and lysosomal markers (Ling et al., 2009, 2014). The vesicles are acidic, a condition that would favor formation of oligomers/aggregates.

7A1a antibody has not been widely used in AD studies, possibly because of a report that it might cross react with tropomyosin. The working examples demonstrate that tropomyosin mRNA was expressed at similarly high levels for all 3 genotypes (RNAseq data), but large amounts of 7A1a positive staining were only observed in edited lines in a progressive manner while staining of unedited parental neurons remained very low and was not progressive. In a Drosophila direct expression model, 7A1a staining specifically recognizes Aβ oligomer/aggregates in several types of endolysosomal vesicle compartments (autophagosomes, endosomes and lysosomes) identified by double staining with vesicle marker specific antibodies (Ling et al., 2009, 2014).

Progressive ND

With increasing culture times 7A1a staining areas progressively increased in both edited genotypes, remained intracellular and appeared to be spatially correlated with pyknotic nuclei. However, Aβ42 cultures always had an earlier appearance of 7A1a staining and the area or positive staining expanded at a faster rate relative to Aβ40 cultures. This difference correlates well with the different rates of neurodegeneration observed in Aβ42 and Aβ40 edited cultures: dead neurons in Aβ42 cultures appeared sooner and accumulated at a faster rate relative to Aβ40 cultures. Since both edited genotypes had equivalent edit specific transcript expression the differential 7A1a staining could be a result of an increased ability of Aβ42 to form oligomers/aggregates or faster Aβ40 removal. Both possibilities are consistent with the differential biochemical properties of these peptides. Pyknosis has been associated with AD disease and is considered a maker for both type 1 and 2 cell death pathways (Ghavami et al., 2014; Nixon and Yang, 2012; Yuan et al., 2003). The results thus suggest that perinuclear intracellular accumulation of oligomeric/aggregated Aβ could be a cause neuronal cell death. The working examples demonstrate that the spatial relationship of 7A1a staining and pyknosis was not strictly dependent on direct Aβ expression but was also observed in unedited control neurons presumably producing Aβ via amyloidogenic APP processing. Unedited cultures did not show progressive 7A1a staining and had only limited ND relative to Aβ edited cultures suggesting a mechanism that can limit widespread damage sufficient to prevent ND. Possibly unedited cells accumulate only low levels of oligomeric Aβ or alternatively the Aβ oligomers may be formed in different vesicle compartments following amyloidogenic or direct Aβ production. This latter possibility could be established with additional experiments co-localizing oligomeric Aβ with specific vesicle markers.

Differentiated cultures are primarily neuronal

AD has a well-known complexity with respect to the range of cell types associated with various disease related observations (De Strooper and Karran, 2016). This cellular complexity can only be partially modeled in culture, primarily using long term organoid differentiation or co-culturing independently differentiated CNS cell types (Arber et al., 2017; Choi et al., 2014; Park et al., 2018). The experimental objectives required not only long-term culture but also reliable and repeatable neuronal differentiation. Preliminary experiments using published protocols designed to generate AD relevant neurons proved too variable (Engel et al., 2016; Wicklund et al., 2010). As demonstrated herein, much better consistency was obtained using a simple embryoid body caudal hindbrain protocol originally developed to enrich for caudal motor neuron (Amoroso et al., 2013). Although many types of neurons are affected in AD, motor neurons are not. However, rostral differentiation of human AD mutant (APPV717I) iPSC cells using a similar differentiation protocol produced a population of neurons with clear amyloidogenic properties and only a modest decrease in amyloidogenic Aβ production relative to rostral differentiation of the same cells (Muratore et al., 2017).

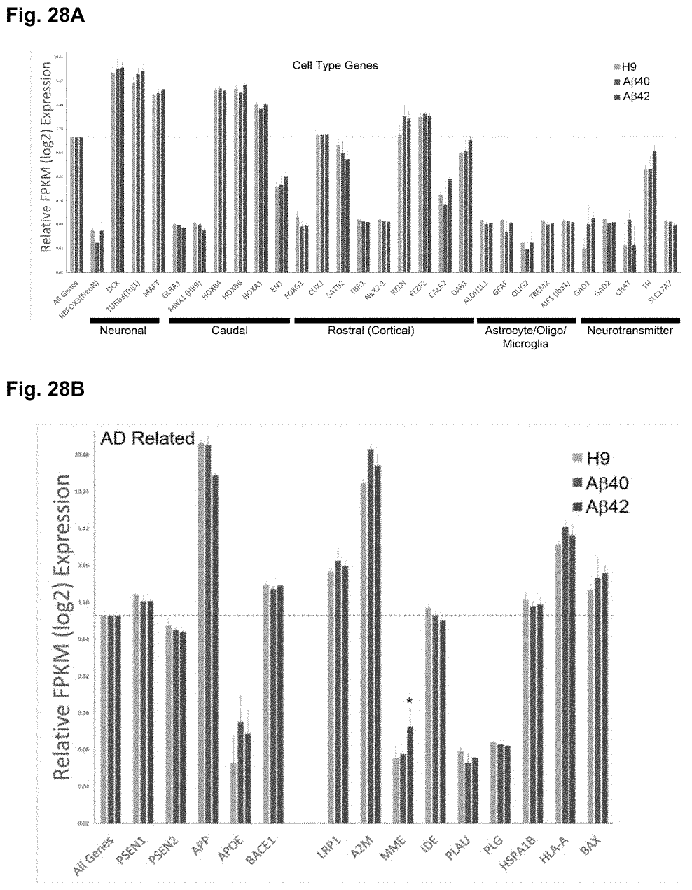

RNAseq data confirmed the caudal nature of differentiated neurons based on expression of cell type specific marker genes. A few rostral marker genes, however, were also expressed. Multiple neurotransmitter phenotype specific genes were detected, but only low levels CHAT suggesting that cholinergic motor neurons are unlikely to be a prominent neurotransmitter phenotype as expected for motor neuron enrichment. Notably, only extremely low or undetectable levels of non-neuronal marker genes (primarily different types of glial cells) were expressed. This is consistent with weekly addition of mitotic blocker. Thus, the observed phenotypes are primarily neuronal and not likely related to other non-neuronal cell types such as microglia or astrocytes known participate in AD primarily through modulation of inflammatory pathways or removal of secreted Aβ. Neither of these processes appears relevant to the disclosed model. RNAseq data for cell specific marker gene expression results are summarized in FIGS. 28A-B.

APOEE3/E4 genotype

The neurons in these studies all have an APOE c3/ c4 genotype. There are potential roles for c4 in intracellular neuronal Aβ retention when Aβ is produced by amyloidogenic processing of APP. Perhaps the primary significance of the c4 allele in the direct Aβ expressing cell lines is also to facilitate intracellular retention. This could also potentially explain the relatively high level of intracellular oligomer accumulation observed relative to only modest intracellular accumulation in other AD culture models of unspecified APOE allele type (Choi et al., 2014; Kondo et al., 2013).

Synaptic deficits

A deficit in synapsin1 stained puncta in 34-day old cultures which was specific for A842 edited cells was observed. This suggests a deficiency in the number of synapses relative to either A840 or unedited cells at this culture age. Synaptic deficits are an early AD phenotype but have rarely been reported in human AD culture models (Mungenast et al., 2016). Other experimental models attribute synaptic deficits to increased amyloidogenic production of Aβ resulting in increased synaptic activity, or to a complex relationship to amyloidogenesis (sometimes involving non-Aβ40 or Aβ42 APP derived proteolytic products) or even a tau dependence (Forner et al., 2017). The synaptic deficiency observed is most likely a result of direct Aβ42 expression and subsequent accumulation of oligomeric/aggregated Aβ42. Unedited cells have only low-level accumulation of Aβ oligomer/aggregates and Aβ40 edited cells have significantly less than Aβ42 cells. Additionally, no significant tau related phenotypes were observed.

Differential gene expression

The small number of DEGs identified (93) generally show a more significant change with greater magnitude in Aβ42 compared to Aβ40 samples. This suggests that common pathways may be involved in these independently edited genotypes and agrees well with the exclusive and/or earlier or more penetrant phenotypic changes in Aβ42 samples at the time of mRNA isolation. It is challenging, however, to relate these expression changes to extensive whole transcriptome expression profiling of AD patient brain samples (or even other iPS cellular AD models). These studies often identify hundreds or thousands of DEGs (Annese et al., 2018; Castillo et al., 2017). This numerical difference with the results is likely due to multiple factors including the simplified rostral neuronal cell type and the isogenic nature of the simplified culture model. Patient samples would contain significant genetic variance that may affect gene expression. They also contain variance in tissue and cell type sampling, co-morbidities, life style differences, penetrance of other AD phenotypes (i.e. tau pathology, non-neuronal inflammatory glial responses, etc.), all of which are likely to affect gene expression. Importantly, the cultures likely represent early changes in neuronal gene expression specific to direct Aβ-expression and accumulation of oligomeric intracellular Aβ.

Despite these comparative challenges, an AD vs non-AD study of temporal cortex samples (Allen et al., 2016) revealed that 88% (82/93) are shared with the DEGs and 51% (42) have the same directional FC. Comparison with hippocampal study (Annese et al., 2018) identified 39% (36) genes with overlap and 67% (24) had the same directional negative FC (i.e. decreased expression).

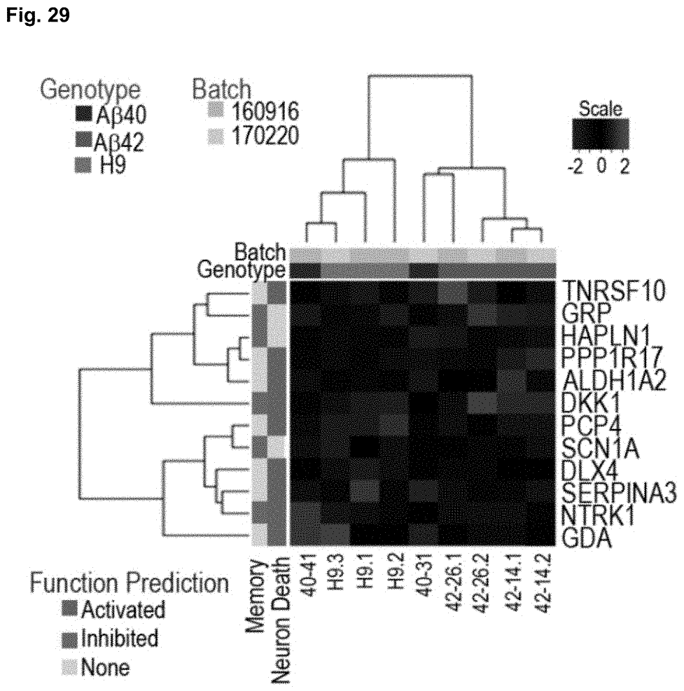

IPA analysis surprisingly identified “Increased Neuronal Loss” and “Decreased Memory” as the top scoring functional processes in the Aβ42 edited samples an obvious relevance to AD. Genes in these two functional processes overlap with each other as illustrated in a clustered sample level heat map (FIG. 29). IPA analysis also suggested involvement of the “Neuroprotective THIOP1 Pathway in AD” as the only significant disease related canonical pathway but only two of the 40 genes were differentially expressed.

A surprising finding was the identification of downregulated genes annotated related to cilia, an organelle not usually associated with AD. Five of these genes, however, were also downregulated in hippocampal AD patient samples (Annese et al., 2018) (DAW1, DNAH11, DNAI2, GDA, TEKT1). Neurons usually contain a primary non-motile cilia (Guemez-Gamboa et al., 2014) believed to function as a major signaling center integrating environmental information through a wide variety of localized G-protein coupled and other types of neuroactive ligand receptors (Berbari et al., 2009). Interestingly, the KEGG “Neuroactive Ligand Receptor Interaction Pathway” was the top scoring pathway using GSEA analysis. Thus, neuroactive signaling may be broadly disrupted in Aβ42 cultures and this may be due to deficient ciliary function.

From the foregoing, it will be appreciated that specific embodiments of the invention have been described herein for purposes of illustration, but that various modifications may be made without deviating from the scope of the invention. Accordingly, the invention is not limited by the following working examples, which are for illustration purposes only.

### EXAMPLE 1. MATERIALS AND METHODS

Genomic Editing: TALEN (Transcription activator-like effector nuclease) pairs were designed to target DNA upstream of the normal APP translation start site using published criteria, their cutting efficiency established in HEK293T cells and used to generate a double strand break in the APP target (Cermak et al., 2011). Donor templates for homology repair contained homology arms flanking the targeted site along with a secretory signal derived from the rat proenkephalin (PENK) gene, a human Aβ40 or Aβ42 coding sequence, and a polyA tail. Donor templates also contained a puromycin selection gene under control of the human phosphoglycerate kinase.

H9 (WiCell WA09) human embryonic stem cells were obtained from the WiCell Foundation and cultured on a feeder free system (Matrigel). Cells were harvested at appropriate confluency and nucleofected with TALEN pairs and donor template using an Amaxa Nucelofector. Nucleofected cells were grown for 48 hours, harvested and plated on puromycin resistant feeder cells at a dilution of 1/30 for 48 hours and then transferred to puromycin drug selection media for two weeks. Approximately ½ of appropriate size colonies were collected for PCR analysis using primer pairs that spanned the flanking DNA and the donor plasmid sequences to confirm insertion of the expression cassette. The stem cell colonies positive for correct size PCR fragments at both the 3′ and 5′ sites were expanded and analyzed for expression of edit specific Aβ340 or Aβ342 expression using qRT-PCR analysis. The forward primer was specific to the rat secretory signal sequence (not present in the human genome) and the reverse primer targets the end of the Aβ40 sequence.

The parental human ES cell line (WA09) was obtained from WiCell Foundation, Madison Wisconsin. All experiments were approved by the City of Hope Stem Cell Oversight Committee (#SC13002). Amyloid Precursor Protein (APP) gene was edited by using a TALEN (Transcription activator-like effector nuclease) pair and a donor template for homologous recombination. TALEN pairs were designed to cleave DNA just upstream of App translation start site as shown schematically in FIG. 1B.

The TALEN design used criteria from Cermak et. al. (2011). The TALEN pair sequences were assembled by using the Golden Gate assembly protocol from Addgene (Golden Gate TALEN and TAL Effector Kit 2.0 # 1000000024). Table 1 below lists TALEN sequences.

TABLE 1
TALEN Sequences

| | |
|---|---|
| TALEN 1 | 5′CGCCTGGCTCTGAGCCCC3′ (SEQ ID |
| | NO: 1) |
| TALEN 2 | 5′GCCGAGGAAACTGAC3′ (SEQ ID NO: 2) |
| TALEN 3 | 5′CGGGCTCCGTCAGTTTCC3′ (SEQ ID |
| | NO: 3) |
| TALEN 4 | 5′CCTCCGCGTGCTCTC3′ (SEQ ID NO: 4) |

Donor templates were constructed with homology arms (HAL=563 bp; HAR=557 bp) from APP gene sequences (ENST00000346798.7, Transcript ID) along with a secretory signal derived from the rat proenkephalin gene (PENK) and human Aβ40 or Aβ42 (Table 2).

TABLE 2
Human Aβ and rat secretory signal sequences

| | |
|---|---|
| Rat preproenkephain | atggcgcagttcctgagactttgcatctggctcgtagcgcttgggtcct (SEQ ID |
| secretory signal | NO: 5) |
| | gcctcctggctacagtgcaggca (SEQ ID NO: 6) |
| Aβ42 | gatgcagaattccgacatgactcaggatatgaa (SEQ ID NO: 7) |
| | gttcatcatcaaaaattggtgttctttgcagaagatgtgggttcaaacaa (SEQ ID |
| | NO: 8) |
| | aggtgcaatcattggactcatggtgggcggtgttgtcatagcg (SEQ ID NO: 9) |
| Aβ40 | gatgcagaattccgacatgactcaggat (SEQ ID NO: 10) |
| | atgaagttcatcatcaaaaattggtgttctttgcagaagatgtgggttcaa (SEQ ID |
| | NO: 11) |
| | acaaaggtgcaatcattggactcatggtgggcggtgttgtcatagcg (SEQ ID |
| | NO: 12) |

To select for edited colonies, puromycin expression driven under PGK (Phosphoglycerate kinase) promoter was used. The sequence fragments for PGK-PURO were obtained from pMSCV puro plasmid (Clontech Cat. No. 634401).

To generate edited H9 cells (Human embryonic stem cells: WiCell WA09), stem cells were cultured on feeder free system (Matrigel). The cells were harvested at appropriate confluency and Nucleofected with TALEN pair and donor template using an Amaxa Nucleofector. The nucleofected cells were allowed to grow on feeder free matrigel for 48 hours. The cells were then harvested and plated on puromycin resistant feeder cells at a dilution of 1/30. The cells were allowed to grow for 48 hours before changing to puromycin selection media. Selection was carried out for two weeks when appropriate size colonies were used for genomic PCR screening. The genomic screening was performed by harvesting ½ of a colony and extracting genomic DNA. This DNA was screened using specific primers spanning the homology arm and specific to the expected insert (primers in Table 3) to confirm correct homologous recombination and insertion at the TALEN cutting site.

TABLE 3
Primer sequences for screening correct genomic editing

| Primers for Genomic Insert Analysis | | | | | |
|---|---|---|---|---|---|
| Target | Forward Primer | Sequence | Reverse Primer | Sequence | Amplicon |
| 5′ Genomic Edit | 5′ Recombinant | GAAGTAAATGGGTTGGCCG | 5′ Recombinant | CCTGAGTCATGTCGGAATT | 712 bp |
| | F | CTTCTTTG | R | CTGCATC | |
| | | (SEQ ID NO: 13) | | (SEQ ID NO: 14) | |
| 3′ Genomic Edit | 3′ Recombinant | GTCGAGGTGCCCGAAGGA | 3′ Recombinant | GGGCAACGATTCAAGAGCG | 934 bp |
| | F | C (SEQ ID NO: 15) | R | A (SEQ ID NO: 16) | |
| Left Homology | LHA F | TTAACGCGGCCGCGGTTCG | LHA R | AGCTGCAGAGATCTAGTCA | 563 bp |
| Arm | | TTCTAAAGATAG | | GCTGATCCGGC (SEQ ID | |
| | | (SEQ ID NO: 17) | | NO: 18) | |
| Right Homology | RHA F | AATATCTGCAGGGTACCAG | RHA R | ATTTCTCGAGTGCTCCTTC | 557 bp |
| Arm | | C GGTAGGCGAG | | CCCCTTCC | |
| | | (SEQ ID NO: 19) | | (SEQ ID NO: 20) | |

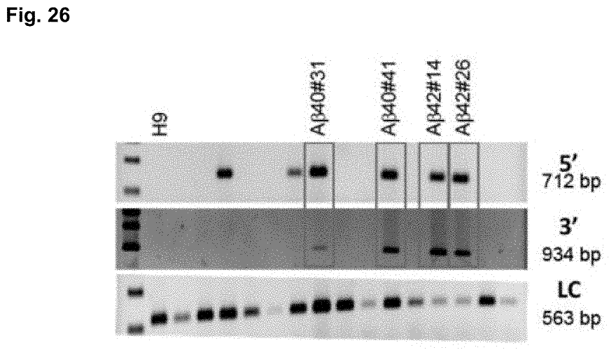

The stem cell colonies with positive PCR amplicon at both ends were further characterized for edit specific Aβ expression using qRT-PCR analysis. Only colonies positive for genomic PCR analysis of both 3′ and 5′ junctions (FIG. 26) and significant secretory Aβ expression were used for phenotypic analysis.

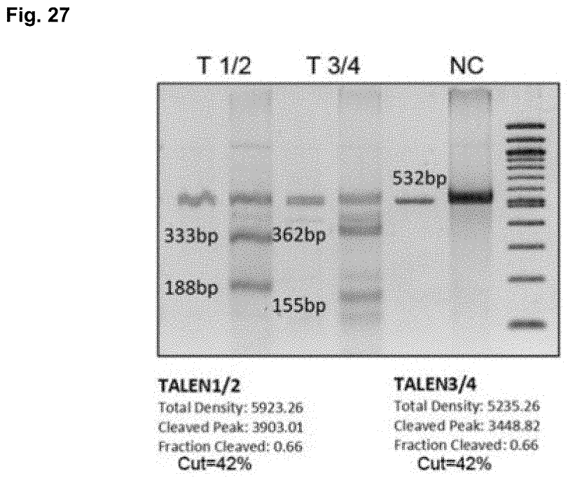

Surveyor Assay: Surveyor Mutation detection kit from Transgenomic (Cat # 706025) was used to detect the efficiency of cleavage by the TALEN pairs. HEK293T cells were transiently transfected by Lipofectamine® 2000 (Invitrogen Cat# 11668-027). After 48 hours of transfection genomic DNA was extracted using QuickExtract™ (DNA extraction solution was from epicentre an Illumina company (Cat# QE09050). The mutant and reference DNA were amplified using primers flanking the cleavage site (look primer file for sequences and conditions). Using thermal cycler Hetro and Homoduplexes were hybridized. Surveyor Nuclease was added to cleave the hybridization products and was used for fragment analysis on Agarose gel electrophoresis. Density of bands was used to determine the efficiency of TALEN cleavage (FIG. 27).

Stem Cell Nucleofection: To increase survival stem cells were treated with 10 μM rock inhibitor (Stemgent Y-27632) at least 1 hour before starting nucleofection. Four wells of confluent H9 stem cells were grown on feeder free matrigel system in media (Stem Cell Technologies: mTeSR1 Cat# 05850) for 4 days. Media were changed daily and differentiating colonies were manually removed. All DNA samples (TALEN pairs and donor template) were purified using endotoxin free kits. Total DNA for TALEN pairs and donor template was ˜5 μg per reaction in a total volume of 10 μl. For each sample 82 μl of Nucleofector® Solution 2 plus 18 μl of supplement 1 were added to make 100 μl of total reaction volume. Nucleofection solution was mixed with TALENs and Donor Template room temperature.

The Amaxa nucleofector was used following their recommended reagents and protocols. Briefly, H9 cells colonies were dissociated into single cells using accutase (7 min at 37° C.), resuspended in ES cell media (DMEM F-12, 20% KO serum, Pen/Strep, NEAA, Glutamax, FGF, β-ME) and pelleted by centrifugation (1000 rpm for 5 min). Cells were resuspend in 1-2 ml of mTeSR medium with rock inhibitor, an aliquot was taken for counting and 1.5×10⁶ cells were used for each nucleofector sample. Cells were pelleted (850 rpm, 115 g, 3 min.), media were removed and the pellet resuspended in 100 μl of RT Nucleofector solution 2 along with the Talen/Donor template mix in a nucleofector cuvette and processed using program B-016 (Human Stem Cells). The sample was recovered in 0.5 ml of pre-warmed mTeSR medium (containing 10 μM Rock inhibitor) and were transferred to a matrigel (BD bioscience Cat #354230) coated 24-well plate. Cells were grown for two days, resuspended and plated on puromycin resistant feeder cells. Puromycin drug selection was initiated after 2 days and maintained for 14 days. All non-nucleofected cells died after two weeks under drug selection.

Cell Culture and Differentiation: ESC culture, embryoid body generation and neuronal differentiation were adapted from a well-established protocol (Amoroso et al., 2013). Briefly, stem cells were grown in gelatin coated six-well plates on an irradiated mouse embryonic fibroblasts feeder layer. Stem cells were maintained in HuES medium which was replaced daily and differentiating colonies were manually removed to maintain pluripotency. Stem cells were passaged weekly and differentiation was initiated ˜1 week after passage using dissociated cells transferred to a 10 cm culture plate for embryoid body (EB) generation. On day 3 cells were grown in Neural Induction Media (NIM) with N2 supplement and 2 μg/ml heparin. On day 5 media was supplemented with ascorbic acid, trans-retinoic acid, Y-27632 ROCK inhibitor, and BDNF. On day 7 smoothened agonist 1.3 was added. Media were replaced every 3rd day and after ˜28-31 days EBs were collected, rinsed with Ca²⁺/Mg²⁺ free PBS, dissociated into individual cells and plated in either 6 or 24 well culture plates precoated with poly-L-ornithine and laminin (1.7×10⁶ or 0.34×10⁶ cells per well) in neural differentiation medium supplemented with 25 μM β-mercaptoethanol and 25 μM glutamate. Cultures were initially treated with 0.5 μM ethynyl deoxyuridine (EdU) for 24 hours and weekly thereafter up to ˜50 days to maintain only post mitotic cells.

ES cell culture and maintenance: ES cell lines (H9 or edited H9) were cultured on gelatin coated (0.1% in PBS) wells with a feeder layer of irradiated mouse embryonic fibroblasts (Fisher Scientific, A34180). Stem cells were cultured in HuES medium (after the second day of plating, media is changed daily). Cells were observed daily and any colonies that start to differentiate were removed using a sterile pipette. ES colonies should have perfect undifferentiated colony morphology before beginning differentiation. Differentiation was started ˜1 week after the previous passage (i.e. cells maintained in culture were passaged -every week). Approximately 8-9 confluent wells of a 6-well dish of stem cells were sufficient to make one 10 cm dish of EB bodies.

EB generation: Day 0: Colonies were released from fibroblast attachment using dispose (1 mg/ml) in PBS. Old medium was aspirated and dispose solution was added, and then incubated in tissue culture incubator until colonies began to detach with gentle tapping (colonies should begin to curl up from the edges and detach from CF-1 Miffs (usually after ˜12-15 minutes, observed at regular intervals with microscope). When colonies were detaching, HuES media were added to stop the dispose digestion. Dissociated colonies were removed by gentle pipetting with a 10 ml pipette and transferred to a 50 ml falcon tube. Cells were let settle for ˜3-5 minutes and washed gently with ˜10 ml PBS (2×). If all the colonies were not released from the plate the above procedure was repeated gently using a cell scraper to increase the yield. In general, ˜95% of colonies were recovered. Triturate released colonies in 1 ml of HuES were supplemented with additional factors. A P1000 pipette was used and triturated ˜7-10 times. The dissociated cells were resuspended in HuES medium supplemented with additional factors (20 ng/ml FGF-2, 10 μM SB431542, 0.2 μM LEN193189 (Stemgent) and 20 μμM Y-27632 (ROCK inhibitor, Stemgent). Pipette cell suspension into a 10 cm culture plate containing 9 ml of HuES with additional factors. The plating concentration was -400,000 cells/ml. This was considered day 0 for EB production.

The following procedure was followed to prepare EBs:

Day 2: change media. Collect cells in a 15 ml tube, centrifuge for 2 min at 100×G (1000 rpm). Aspirate media and replace with fresh HuES +factors medium. (Do not pipette up and down to keep small intact EB).

Day 3: collect as above and change to Neural Induction Media 1(NM 1).

Day 5: change media to NIM 2. (NM1+AscoRabbitic Acid (AA, Sigma) 0.4 μg/μl, + all trans-retinoic acid (RA, Sigma) 1 μM and -Y-27632 ROCK Inhibitor).

Day 7: change media to NIM3 (NIM1+SAG, -βFGF).

Day 9-15: NIM+BDNF+AA+RA+SAG as above, Change every 3 days.

Day 17 to 28-31 days (most often 28d): Switch to NDM 1 medium changed every 3 days. Maintain all supplements as above plus: B-27 (50x, Invitrogen), GDNF (10 ng/mL, R&D systems), CNTF (10 ng/mL, R&D systems), IGF-1 (10 ng/mL, R&D systems).

The following procedure was followed for neural differentiation:

Dissociate differentiated EBs at day 20-31 (generally at 28 days).

Collect EBs in 50 ml Falcon tube and allow to settle for 5 minutes.

Rinse EBs and let settle in CMF (Calcium and Magnesium free PBS).

Dissociate into individual cells using 2 ml of 0.25% Trypsin-EDTA (GIBCO 25200-056).

Incubate at 37° C. for ˜6-7 min while gently swirling in 37° C. water bath.

Add an equal volume FBS to stop the trypsin digestion and then 8 ml of CTWM.

Centrifuge 3 min at 1400 rpm (IEC Centra CL2 centrifuge).

Remove supernatant and resuspend pellet in 1 ml CTWM by trituration ˜7 passes with P1000 pipette and add 10 ml CTWM.

Pass cells through a 40 μm strainer to remove large clumps (Sigma-Corning CLS431750-50EA) into a new 50 ml tube.

Take a sample for cell counting.

Centrifuge at 1600 rpm.

Resuspend single cell suspension in NDM medium+all supplements+β-ME (25 μM)+Glutamate (25 μM)

Seed dissociated cells at 1.7×10∧6 per well in a 6 well plate or 0.34×10∧6 per well in a 24 well plate coated with poly-L-ornithine and laminin. Culture with EdU to maintain only post-mitotic cells.

Unused cells can be cryopreserved by freezing in 2× freezing medium (Chemicon, for ES cells) with Frosty chambers (Fisher) optional.

Media recipes are provided below. BDNF, GDNF, IGF-1, CNTF lyophilized powders were dissolved in 0.1% BSA that had been filtered through 0.22 um filter. BDNF 10 ug powder+200 ul BSA=50 μg/ml stock, GDNF 10 ug powder+200 ul BSA=50 ∥g/ml stock. IGF-1 100ug powder+2ml BSA=50 μg/ml stock. CNTF 20 ug powder+1ml BSA=20 μg/ml stock. Ascorbic Acid powder was mixed with filtered, autoclaved DDW. Retinoic acid powder was dissolved in DMSO and protected from light. To make 1 mM stock, 10 μl of 100 mM was added to 990 μl of DMSO. To make 100 mM from powder, 1.67 ml DMSO was added to 50 mg.

TABLE 4
HuES Medium

| Final Concentration | Volume for 10 ml solution | |
|---|---|---|
| 80% DMEM-F12 | 7.67 | ml |
| 20% KO Serum Replacer | 2 | ml |
| 50 U and 50 mg/ml Pen/Strep soln. 100X | 100 | μl |
| stock | | |
| 1% Non-essential Amino Acids 100X stock | 100 | μl |
| 1 miM L-Glutamax 100X stock | 100 | μl |
| 0.1 mM β-mercaptoethanol 1000X stock | 10 | μl |
| 20 ng/ml β-FGF (10 μg/mL stock) | 20 | μl |

β-ME and βFGF were added fresh on the day of use. Other components were mixed together and stored in refrigerator until use. β-mercaptoethanol: 7 ul of 14.3 M β-ME+1 ml calcium and magnesium free PBS. (Protected from light and a new stock was made every week stored at −20° C.). βFGF: 50 μg was dissolved in 5 ml Knock-Out Serum (Life Technologies 10828-028) to make a 10 μg/ml stock.

TABLE 5
EB1 (HuES +βME, βFGF, ROCK Inhibitor, SB431542)

| | | Volume |
|---|---|---|
| Final Concentration | Stock | for 10 ml solution |
| 80% DMEM-F12 | 1x | 7.65 ml |
| 20% KO Serum Replacer | 1x | 2 ml |
| 50 U and 50 mg/ml pen/strep soln | 100x | 100 μl |
| 1% Non-essential Amino Acids | 100x | 100 μl |
| 1 mM β-Glutamax stock | 100x | 100 μl |
| 0.1 mM β-mercaptoethanol | 100 mM | 10 μl |
| 20 ng/ml βFGF | 10 μg/ml | 20 μl |
| 10 μM Y-27632 (ROCK inhibitor | 10 mM | 10 μl |
| 10 μM SB431542 | 10 mM | 10 μl |
| 0.2 μM LDN193189 | 10 mM | 0.2 μl |

βFGF helps with single cell survival, SB and LDN are “sternness” inhibitors.

TABLE 6
NIM1 (Neural Induction Medium, EB1 media - K0 serum,
−bME + heparin)

| | | Volume |
|---|---|---|
| Final Concentration | Stock | for 10 ml solution |
| 100% DMEM-F12 | 1x | 9.55 ml |
| 50 U and 50 mg ml-1 pen/strep soln | 100x | 100 μl |
| 1% Non-essential Amino Acids | 100x | 100 μl |
| 1 mM L-Glutamax | 100x | 100 μl |
| 1 × N2 Supplement | 100x | 100 μl |
| 2 μg/ml Heparin | 2 mg/ml | 10 μl |
| 20 ng/ml βFGF | 10 μg/ml | 20 μl |
| 10 μM Y-27632 (ROCK inhibitor) | 10 mM | 10 μl |
| 10 μM SB431542 | 10 mM | 10 μl |
| 0.2 μM LDN193189 | 10 mM | μl |

TABLE 7
NIM 2 (NIM1 - ROCK Inhibitor, +AA, +RA)

| | | Volume for 10 ml |
|---|---|---|
| Final Concentration | Stock | solution |
| DMEM-F12 | 1x | 9.56 ml |
| 50 U and 50 mg ml-1 Pen/Strep soln | 100x | 100 μl |
| 1% Non-essential Amino Acids | 100x | 100 μl |
| 1 mM L-Glutamax | 100x | 100 μl |
| 1 × N2 Supplement | 100x | 100 μl |
| 2 mg/ml Heparin | 2 mg/ml | 10 μl |
| 10 ng/ml BDNF | 50 μg/ml | 2 μl |
| βFGF 20 ng/ml | 10 μg/ml | 20 μl |
| 0.4 mg/ml AscoRabbitic acid | 10 mg/ml | 0.4 μl |
| 1 μM RA | 1 mM | 10 μl |

TABLE 8
NIM 3 (NIM2 +SAG, −FGF)

| | | Volume for 10 ml |
|---|---|---|
| Final Concentration | Stock | solution |
| DMEM-F12 | 1x | 9.56 ml |
| 50 U and 50 mg ml-1 pen/strep soln | 100x | 100 μl |
| 1% Non-essential Amino Acids | 100x | 100 μl |
| 1 mM L-Glutamax | 100x | 100 μl |
| 1 × N2 Supplement | 100x | 100 μl |
| 2 mg/ml Heparin | 2 mg/ml | 10 μl |
| 10 ng/ml BDNF | 50 μg/ml | 2 μl |
| 0.4 mg/ml AscoRabbitic acid | 10 mg/ml | 0.4 μl |
| 1 μM RA | 1 mM | 10 μl |
| 2 mM SAG | 10 mM | 2 μl |

TABLE 9
NDM 1 (Neural Differentiation Medium)

| | | |
|---|---|---|
| | | Volume for 10 ml |
| Final Concentration | Stock | solution |

| | | | |
|---|---|---|---|
| Neural Basal Medium | 1x | To 10 | ml |
| 50 U and 50 mg m1⁻¹ pen/strep soln | 100x | 100 | μl |
| 1% Non-essential Amino Acids | 100x | 100 | μl |
| 1 mM L-Glutamax | 100x | 100 | μl |
| 1 × N2 Supplement | 100x | 100 | μl |
| 10 ng/ml BDNF | 50 μg/ml | 2 | μl |
| 0.4 μg/ml AscoRabbitic acid | 10 mg/ml | 0.4 | μl |
| 1 μM RA | 1 mM | 10 | μl |
| 2 μM SAG | 10 mM | 2 | μl |
| 1x B27 supplement | 50x | 200 | μl |
| 10 ng/ml GDNF | 50 μg/ml | 2 | μl |
| 10 ng/ml CNTF | 20 μg/ml | 5 | μl |
| 10 ng/ml IGF-1 | 50 μg/ml | 2 | μl |

Before dissociating cells coverslips were coated with poly-L-ornithine and laminin for 2 days. Poly-L-ornithine—(coated first) stock was 50 mg/ml: diluted with COLD DMEM/F12, working concentration was 0.1 mg/ml (2.0 μl for 1 ml). Incubated overnight at 37° C. To make stock from powder: 100 mg was dissolved in 2 ml of autoclaved DDW and passed through 0.22 μm filter to make 50 mg/ml stock. Laminin—(coated second) stock was 1 mg/ml: diluted with COLD DMEM/F12, working concentration was 20 μg/ml (20ul per 1 ml). Incubated overnight at 37° C.

TABLE 10
CTWM (Complete Trituration Wash media, ~20 ml per genotype)

| | | Volume for 100 ml |
|---|---|---|
| Final Concentration | Stock | solution |
| 1x PBS (Calcium Magnesium Free) | 1x | 89.1 ml |
| 25 mM Glucose | 1 M | 2.5 ml |
| 01% dialyzed BSA | 4% | 2.5 ml |
| N2 supplement | 100x | 1 ml |
| B27 supplement | 50x | 2 ml |
| MgCl2 2 mM | 1M | 200 μl |
| EDTA 1 mM | 0.5M | 200 μl |
| FBS | 1x | 2.5 ml |

TABLE 11
NDM (Neural Differentiation Medium)

| | | Volume |
|---|---|---|
| Final Concentration | Stock | for 10 ml solution |
| Neural Basal Medium | 1x | 9.38 ml |
| 50 U and 50 mg ml⁻¹ pen/strep soln | 100x | 100 μl |
| 1% Non-essential Amino Acids | 100x | 100 μl |
| 1 mM L-Glutamax | 100x | 100 μl |
| 1 × N2 Summplement | 100x | 100 μl |
| B27 supplement | 50x | 200 μl |
| 10 ng/ml BDNF | 50 ug/ml | 2 μl |
| 10 ng/ml GDNF | 20 ug/ml | 2 μl |
| 10 ng/ml IGF-1 | 50 ug/ml | 2 μl |
| 10 ng/ml CNTF | 20 ug/ml | 5 μl |
| 0.4 μg/ml AscoRabbitic acid | 10 mg/ml | 0.4 μl |
| 25 μM Glutamate | 25 mM | 1 μl |
| 25 μM β-mercaptoethanol | 100 mM | 2.5 μl |
| 1 μM RA | 1 mM | 10 μl |
| 0.5 μm EdU | 5 mM | 1 μl |

25 mM glutamate (Sigma G8415-100G, M.W. 147.13) stock was prepared by dissolving 0.11 g in 30 ml of 1N HCL. EdU (mitotic poison) was used to keep dividing cells from over-growing the culture. A 5 mM stock solution was diluted in NDM to a final concentration of 0.5 μm. The initial EdU treatment was for exactly 24 hours before changing back to NDM with no EdU. After this initial treatment, cultures were treated with EdU for 24 hours every week until cultures are approximately 50 days post-EB dissociation.

qRT-PCR: Total RNA was extracted using the RNeasy Micro Kit from (Qiagen) following the manufacturer's protocol. RNA concentration and purity were determined spectrophotometrically and cDNA prepared using qScript cDNA SuperMix (Quanta) following the manufacturers protocol. All reactions were carried out in a 20 μl reaction mixture containing 12.5 μl iQTM SYBR® Green Supermix (Bio-Rad), 2 μM of each forward and reverse primers, 0.25 μg cDNA, and DEPC-Treated Water (Ambion) to adjust the final volume to 20 μL. Amplification was carried out using a BioRad CFX96 Touch™ Real-Time PCR machine in clear 96 well sealed plates and data was collected and analyzed using BioRad CFX Manager (v3.1).

Total RNA was isolated from cells using RNA STAT60 RNA extraction reagent from Amsbio or Zymo Research. Where necessary RNA was concentrated using glycogen precipitation. For cDNA synthesis RETROscript® Reverse Transcription Kit (AM1710) or Quanta (Qiagen) was used. The cDNA was amplified using oligo (dT) primers. The cDNA generated was diluted and used for qRT-PCR analysis with iQTM SYBR Green Supermix from BIORAD (1708880) in CFX96 machine. Mean normalized expression values were calculated relative to GAPDH reference gene. Cycling Conditions were: Denaturation/enzyme activation 95° C. for 3 minutes; Denature 95° C., 30 seconds; annealing and extension 55° C. for 20 seconds, 40 cycles. Melting curve: 55° C. to 95° C. for 5 seconds in 0.5° C. increments to ensure a single amplicon.

TABLE 12
qRT-PCR Primers

| Target | Forward (5′→3′) | Reverse (5′→3′) |
|---|---|---|
| GAPDH | ATGGGGAAGGTGAAGGTC | GGGATTTCCATTGATGACAAGCTTCCC |
| | GGAGTC (SEQ ID NO: 21) | G (SEQ ID NO: 22) |
| Edited Rat SS-Aβ* | ATGGCGCAGTTCCTGAGA | ATGATTGCACCTTTGTTTGAACC (SEQ |
| | (SEQ ID NO: 23) | ID NO: 24) |
| Edited Rat SS-Aβ42** | ATGGCGCAGTTCCTGAGA | CGCTATGACAACACCGC (SEQ ID |
| | (SEQ ID NO: 25) | NO :26) |
| APP Exon 1/2 | GTTTGGCACTGCTCCTGCT | CTGATGGATCTGAATCCCACTTCCC |
| | G (SEQ ID NO: 27) | (SEQ ID NO: 28) |
| APP Exon 5/6 | GAGGAGGATGACTCGGATG | GGTGGTTCTCTCTGTGGCTTCTTCGT |
| | TCTGG (SEQ ID NO: 29) | (SEQ ID NO: 30) |
| APP Exon 16/17 | GATGCAGAATTCCGACA | CCATGATGAATGGATGTGTAC (SEQ ID |
| | (SEQ ID NO:31) | NO: 32) |
| RAB5 | CTAGTGCTTCGTTTTGTGAA | CATATACAACTATGGCTGCTTGTG |
| | AGG (SEQ ID NO: 33) | (SEQ ID NO: 34) |
| LAMP1 | CTACTGCTGTTGCTGCTGCT | GGCATCTGATGGCAGGTCAAAGG |
| | CG (SEQ ID NO: 35) | (SEQ ID NO: 36) |
| LC3B | ATGCCGTCGGAGAAGACC | CTTGATGAGCTCACTCATGTTGACATG |
| | TTCAAGCAG (SEQ ID NO: 37) | GTCAGG (SEQ ID NO: 38) |
| VAMP7 | CTGGAGGTGACAGAGCAGA | TGTCTGTGCTCTTGAACCGT (SEQ ID |
| | TT (SEQ ID NO: 39) | NO: 40) |
| RAB11 | GACGACGAGTACGACTACC | GCAAACTCTACTCCAATGGTGC (SEQ |
| | TC (SEQ ID NO: 41) | ID NO: 42) |
| PICALM | AGCAAGTACATGGGGAGAT | TTAAGGCCAGCTGAAGGGTG (SEQ ID |
| | GC (SEQ ID NO: 43) | NO: 44) |
| RAB3A | AAAGTCACCGCCGCTAGG | ACCTTGAAGTCGATGCCCAC (SEQ ID |
| | (SEQ ID NO: 45) | NO: 46) |

*Rat secretory signal sequence (Forward) and human Aβ40 or 42 (Reverse)
**Ab42 edit specific reverse primer

Microscopy, Immunocytochemistry, Live-Dead Analysis and Image Analysis: Fluorescence samples were observed with a Zeiss Axio Observer microscope (Xenon illumination) using either a 20X NA=0.80 plan-apochromat objective or a 40x or 63x plan-apochromat objective (NA=1.4, Oil). Optical Z sections were acquired with a Zeiss Axiocam506 camera using Zeiss Zen Blue microscope control software (SP2). Unstained cultures were observed using a Nikon Diaphot inverted microscope equipped with Hoffman modulation contrast objectives (HMC EF 10X NA=0.25 or HMC 20X LWD NA=0.4) and images were obtained with a SPOT RT230 cooled CCD camera operated by SPOT Advanced Imaging Software. Image analysis used semi or fully automated macros implemented in the FIJI version of NIH ImageJ (v1.46 or 2) (Schindelin et al., 2012). For visual clarity some images were adjusted for brightness and contrast using Adobe Photoshop (CS4 or CS5). Due to variability in the number of cells in neuronal clusters both among genotypes differentiated in parallel, as well as across independent differentiations, quantitative data were usually normalized to the number or area of DAPI staining.

Microscopy: Fluorescent immunocytochemically stained cells were observed with a Zeiss Axio Observer microscope (Xenon illumination) using either a 20X NA=0.80 plan-apochromat objective or a 40× or 63× plan-apochromat objective (NA=1.4, Oil). Optical Z section images were acquired with a Zeiss Axiocam506 camera (0.05-1 μm spacing, total focal range spanning the thickness of the cultured cells, usually -10-20 μm) using Zeiss Zen Blue microscope control software (SP2). Unstained cultures were observed using a Nikon Diaphot inverted microscope equipped with Hoffman modulation contrast objectives (HMC EF 10X NA=0.25 or HMC 20X LWD NA=0.4) and images were obtained with a SPOT RT230 cooled CCD camera operated by SPOT Advanced Imaging Software. For fluorescence live/dead analysis and accompanying modulation contrast images, Z sections were acquired at ˜1 μm spacing spanning the whole culture depth. All image analysis was performed using semi or fully automated macros implemented in the FIJI version of NIH ImageJ (v1.46 or 2) 30. For visual clarity some images are adjusted for brightness and contrast using Adobe Photoshop (CS4 or CS5).

Image Analysis: Image analysis was performed using the FIJI version of NIH ImageJ (v1.46 or 2) (imagej.netiFiji). Control images were collected for samples with no addition of either primary antibody or secondary antibody.

DAPI: Due to variability in the size of neuronal clusters both within a particular differentiation across the different genotypes as well as across independent differentiations, quantitative data were normalized to the number of DAPI stained nuclei. Briefly, the DAPI channel was processed using the rolling ball (radius=50) background subtract function, autothresholded (Otsu method), converted to an inverted binary image and individual nuclei were estimated following a dilation and watershed operation. Counts were obtained with the analyze particles function using a filter size >30 μm2 and a circularity between 0 and 0.85, excluding nuclei on the edges of the image. In some cases, 2-3 optical sections were combined using a maximum intensity Z projection of individual adjacent optical sections.

DCX and NeuN: Because of their complex shapes, doublecortin stained neurons were counted manually using the green cannel from the corresponding optical section or Z projection. NeuN quantification used the red cannel background subtracted images (rolling ball radius=400, size=60 μm, circularity 0-0.9). Multiple neuronal clusters were analyzed for three independent differentiations for each genotype and normalized to the number of DAPI stained nuclei.

Aggregated Aβ: Oligomeric/aggregated Aβ (7A1a) quantification was done by creating a tightly bound box around each individual neuronal cluster and cropping the image to this area. Background was subtracted using a 150-pixel rolling ball radius. A maximum intensity projection was then created from an image stack (0.5 μm Z intervals, 16-60 images), then using the Z-projection maximum intensity algorithm. The individual color channels were separated and the Aβ channel was thresholded using the IsoData method to measure the area of positive staining within the neural cluster and expressed as a percentage of the total cluster area. Neuronal cluster area was measured from Hoffman interference contrast images by using the freehand tool to manually trace the edges of clusters and using the measure area function.

Synapsin 1: Synapsin1 puncta quantification was done by subtracting the background using a 150-pixel rolling ball radius and creating a maximum intensity projection of a stack of images (0.5 μm sections, 11-31 images). The nuclei were counted the same way as in the DCX and NeuN analysis and the synpasin1 puncta were counted using the ImageJ find maxima function. The number of synapsin1 puncta were then normalized to the number of DAPI nuclei present in each NC.

Antibody Staining: Cells were grown on polyornithine/laminin coated 15 mm No. 1 glass coverslips (Fisher Scientific) placed in 6 well plates. Cells were fixed with 4% paraformaldehyde for 20 minutes followed by washing in PBS (3×, 5 min.) and coverslips were stored in 0.03% NaN3 in PBS at 4° C. until observation. Coverslips were incubated with blocking buffer (0.3% Triton X-100 and 5% Bovine Serum Albumin in PBS) for ˜2 hr. at room temperature, washed briefly with PBS and incubated overnight at 4° C. with primary antibody diluted in 0.3% Triton X-100, 1% bovine serum albumin in PBS (antibody dilution buffer). Coverslips were washed with PBS (3×, 5 min.) with antibody dilution buffer and incubated with fluorescent labeled secondary antibodies for two hours at room temperature, washed with PBS, incubated with DAPI (1 μg/μl) for 5 minutes at room temperature, washed with PBS (2×, 5 min.) and mounted onto glass slides using DAKO Fluorescent Mounting Medium. Additional coverslips were stained after eliminating either the primary or secondary antibody to serve as negative staining controls.

Lamp1, Rab5, Rab3A, LC3B: Coverslips from 38-43 or 63-day old cultures were stained with anti-Lamp 1, anti-Rab5, anti-Rab3A or anti-LC3B antibody. Particle counts were measured following background subtraction (rolling ball radius=15 pixels) and auto thresholding using a maximum intensity Z projection of two or three 1 μm spaced optical sections with the analyze particles function (size filter=0.05-2 μm2) and normalized to the number of DAPI stained nuclei.

TABLE 13
Antibodies

| Antibody Name | Type | Supplier | Cat. Number | Concentration | |
|---|---|---|---|---|---|
| PRIMARY ANTIBODIES: | | | | | |
| Anti-Doubiecortin | Rabbit | Abcam | ab104224 | 1 | μg/ml |
| | polyclonal | | | | |
| Anti-NeuN | Mouse | Abcam | ab104224 | 2 | μg/ml |
| | monoclonal | | | | |
| 7A1a (Anti-Aβ | Mouse | New England | ALI 2b13 | 1.4 | μg/ml |
| oligomeric/aggregate specific | monoclonal | Rare | | | |
| | | Reagents | | | |
| Anti-Aβ 6E10 | Mouse | Covance | SIG-39320 | 1 | mg/ml |
| | monoclonal | | | | |
| Anti-Tuj1 (anti-beta III tubulin) | Mouse | Abcam | ab78078 | 1 | μg/ml |
| | monoclonal | | | | |
| Anti-Synapsin 1 | Rabbit | Abcam | ab8 | 0.2 | μg/ml |
| | polyclonal | | | | |
| Anti-LC3B | Rabbit | Cell Signaling | | 1:400 | dilution |
| | polyclonal | | | | |
| Anti-Rab3A | Rabbit | Abcam | ab3335 | 1:200 | dilution |
| | polyclonal | | | | |
| Anti-Reb5 | Rabbit | Abcam | ab18211 | 1 | μg/ml |
| | polyclonal | | | | |
| Anti-Lamp 1 | Rabbit | Abcam | ab24170 | 0.25 | μg/ml |
| | polyclonal | | | | |
| Anti-Nestin | Rabbit | Abcam | ab92391 | 1:250 | dilution |
| | polyclonal | | | | |
| Anti-Phospho-tau(S422) | Rabbit | Abcam | ab79415 | 1:200 | dilution |
| | polyclonal | | | | |
| Anti-Oct4 | Goat | Abcam | ab27985 | 2 | μg/ml |
| | polyclonal | | | | |
| Anti-GFAP | Chicken | Abcam | ab48050 | 1:500 | dilution |
| | polyclonal | | | | |
| Anti-Choline acetyitransferase | Rabbit | Abcam | ab68779 | 1:500 | dilution |
| | polyclonal | | | | |
| Anti-Rab4 | Rabbit | Abcam | ab 109009 | 1:500 | dilution |
| | monoclonal | | | | |
| SECONDARY ANTIBODIES: | | | | | |
| anti-Chicken IgY H&LAlexa Flour | Goat | Abcam | ab150169 | 3.9 | μg/ml |
| 488 | polyclonal | | | | |
| anti-Rabbit IgG H&L Alexa Flour | Goat | Abcam | ab150077 | 2 | μg/ml |
| 488 | polyclonal | | | | |
| anti-Mouse IgG H&L Alexa Flour | Goat | Invitrogen | a11005 | 2 | μg/ml |
| 594 | polyclonal | | | | |

Live-Dead Analysis: Neuronal viability was estimated by measuring the relative proportion of live/dead cells in neuronal clusters grown on coverslips or directly in culture wells using a commercial fluorescence assay (ThermoFisher LIVE/DEAD™ Viability/Cytotoxicity Kit, for mammalian cells, #L322) according to the manufacturer's directions.

Live/dead analysis was done by staining neuronal clusters growing on coverslips or in 12 well plates using ethidium homodimer (red fluorescence, dead cells) and calcein AM esterase substrate (green fluorescence when hydrolyzed) (ThermoFisher, #R37601). Three to five individual focal planes were obtained along with a corresponding Hoffman contrast image. The number of fluorescent pixels in an area was measured by separating individual color channels of the stack, subtracting background using a 50-pixel rolling ball radius and creating a maximum intensity projection using auto brightness and contrast to threshold each channel. Data is presented as percent red area relative to the total area of red plus green channels. Hoffman modulated contrast images were obtained as a Z-stack with a spacing of ˜1 μm and processed using the simple EDF wavelet processing Plugin in FIJI (Easy setting, medium quality) to construct an extended depth of field image.

RNA-Seq: Stem cells were differentiated for 36 or 38-days and total RNA was extracted using the RNeasy Micro Kit (Qiagen) following the manufacturer's protocol. RNA concentration and purity were determined using a NanoDrop ND-1000 spectrophotometer and processed for RNA-Seq analysis by the City of Hope Genomic Core Facility. The sequencing data files have been deposited in the NIH GEO database (GSE119527).

Reads were aligned against the human genome (hg19) using TopHat2 (Kim et al., 2013). Read counts were tabulated using htseq-count (Anders et al, 2015) with UCSC known gene annotations (TxDb.Hsapiens.UCSC. hg19. knownGene Hsu et al., 2006). Fold-change values were calculated from Fragments Per Kilobase per Million reads (FPKM, Mortazavi et al., 2008) normalized expression values, which were also used for visualization (following a log2 transformation). Aligned reads were counted using GenomicRanges (Lawrence et al., 2013). P-values were calculating from raw counts using edgeR (Robinson et al., 2010), and false discovery rate (FDR) values were calculated using the method of Benjamini and Hochberg (1995). Prior to p-value calculation, genes were filtered to only include transcripts with an FPKM expression level of 0.1 (after a rounded log2-transformation) in at least 50% of samples (Warden et al., 2013) as well as genes that are greater than 150 bp.

Two differential steps were used to define differentially expressed genes. First, genes that vary between Aβ42 and parental H9 cells were identified using a 2-variable differential expression model (Aβ42 status, and run/batch), with the initial set of genes were identified as differentially expressed if they had a |fold-change| >1.5 and FDR <0.25. Then, a set of differentially expressed genes between Aβ40 versus parental H9 were identified as differentially expressed if they could be identified with a more liberal criteria |fold-change| >1.2, and unadjusted p-value <0.05); if there was overlap between genes with the same fold-change sign, those overlapping genes were filtered. The remaining genes were then used for visualization in a batch/run-centered heatmap (expression centered by batch, prior to setting the per-gene expression to have a mean to and standard deviation to 1), using heatmap.3 (github.com/obigriffith/biostar-tutorials/blob/master/Heatmaps/heatmap.3.R) and Pearson's Dissimilarity as the distance metric.

Gene Ontology (GO, Ashburner et al., 2000) enrichment was calculated using goseq (Young et al., 2010). Systems-level analysis was performed in IPA (Ingenuity® Systems, www.ingenuity.com) and GATHER (Chang et al., 2006)).

Statistical Analysis: Prism (v7, Graph Pad) was used for statistical analyses (descriptive statistics, ANOVA, variance estimates and correlation) and graphic preparation.

### EXAMPLE 2. GENERATION OF MODIFIED STEM CELLS

Using the approach described above and illustrated in FIG. 1A, modified embryonic stem cells expressing a secretory form of Aβ₁₋₄₂ or Aβ₁₋₄₀ were produced using TALEN-based genome editing of H9 human embryonic stem cells. TALEN genome editing was used to modify a single allele of APP in H9 human embryonic stem cells. Two different sets of TALEN pairs targeted sites in the first exon upstream of the normal translation start site. Double strand breaks were repaired via homologous recombination in the presence of a plasmid containing either Aβ₁₋₄₂ or Aβ₁₋₄₀ coding sequence preceded by a secretory signal sequence as shown schematically in FIG. 1A. The modified allele is under regulatory control of the APP promoter, but expresses an Aβ coding sequence directly in place of APP. A secretory signal sequence was included in the human embryonic stem cell editing plasmid to ensure peptide production was routed through the normal cellular secretory pathway, similar to APP.

TALEN genomic editing was used to modify the normal wild-type APP gene in WiCell WA09 (H9) human embryonic stem cells (hES). This cell line was chosen because of its widespread use in stem cell studies, the availability of many well characterized neuronal differentiation protocols and because the APOE genotype contains one copy of an c4 allele which is the major genetic risk factor for sporadic Alzheimer's disease (SAD) (Genin et al., 2011). The APOE genotype (c4/c3) was confirmed using allele specific PCR analysis. The editing strategy is shown schematically in FIG. 1C TALEN pairs were designed to induce a double strand break (DSB) within the first exon of the App locus upstream of the normal App transcriptional start site. The DSB was repaired by homologous recombination in the presence of donor plasmids that contained a secretory signal sequence derived from the rat preproenkephalin gene (PENK, Rattus norvegicus) fused in frame to either a human Aβ40 or Aβ42 coding sequence and followed by a polyA tail just upstream of a puromycin drug selection gene. This insertion cassette was flanked by left and right homology arms to direct insertion into the normal App locus.

Successful editing resulted in inactivation of the modified App allele and its replacement with direct expression of either secretory Aβ40 or Aβ42. Importantly, the parental and edited cell lines are essentially isogenic ensuring that phenotypic differences are directly attributable to the specific edits. The rat PENK secretory signal sequence is not present in the human genome allowing PCR analysis to specifically detect edited Ab transcripts. Following translation, the signal peptide is completely removed by normal secretory pathway processing resulting in direct production of either an Aβ40 or Aβ42 peptide (Abramowski et al., 2012; Iijima et al., 2004) eliminating any requirement for amyloidogenic APP processing by β and γ secretases. Since the edits are introduced directly into the normal APP locus, expression is under control of the normal APP regulatory DNA. This distinguishes the model disclosed herein from others that generally used exogenous promoters to drive overexpression. This model could potentially speed up proteotoxic Ab accumulation on a time scale suitable for working with cultured human neurons while potentially minimizing overexpression artifacts.

Proper editing was initially identified by PCR screening of multiple subclones using 3′ and 5′ specific primers and confirmed by genomic sequencing. Since subcloning as well as TALEN editing has the potential to generate off-target effects (primarily indels) or other mutations, although at extremely low levels (Woodruff et al., 2013), two independently isolated subclones for each edited genotype were phenotypically characterized in parallel. No consistent phenotypic differences between subclones were observed, suggesting that the differences are genotype specific (i.e. due to direct expression of either Aβ40 or Aβ42). All edited cell lines used in this study were heterozygous for the edit ensuring that normal APP will still be expressed from the unedited allele.

### EXAMPLE 3. SEQUENCE ANALYSIS OF MODIFIED EMBRYONIC STEM CELLS EXPRESSING AN AMYLOID BETA SECRETORY PEPTIDE.

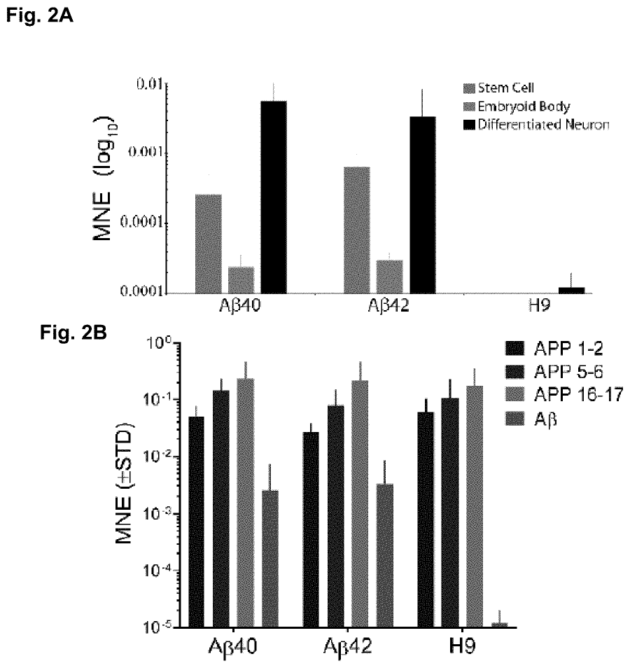

A plurality of independent clones modified for Aβ₁₋₄₂ or Aβ₁₋₄₀ have been confirmed by analysis of the 3′ and 5′ insertion sites. Clones have been analyzed for specific modified Aβ transcription using qRT-PCR primers that can distinguish the edits. Results of APP promoter activity at different stages following neuronal differentiation of parental H9 cells as well as modified H9 cells (FIGS. 2A-B) establish increasing expression as cells transit from a stem cell state to fully differentiated neurons (Amoroso et al. 2013). Expression levels increased more than five-ten fold in differentiated neurons relative to stem cells. The levels of App expression were not less than the expected 50% reduction expected from modification of a single App allele in differentiated neurons using a set of primers probing different extents of the molecule (FIGS. 2A-B).

qRT-PCR was used to measure edit specific expression of secretory tagged Ab. The forward primer was specific to the rat PENK secretory signal peptide which is absent from the human genome and a reverse primer to the end of the Aβ40 sequence which is present in both edits. No edit specific transcripts were detected in unedited H9 cells (FIG. 2A). Significant levels of direct Aβ expression were found in undifferentiated stem cells, EB stage cells or differentiated neurons. The relative expression levels were similar for both edited genotypes at these three developmental stages indicating that they are under the same transcriptional regulatory control. Additionally, it was confirmed that only secretory tagged Aβ42 expression could be detected in Aβ42 edited lines using a reverse primer specific to the unique 5′ nucleotides in Aβ42. Undifferentiated stem cells show an intermediate expression level, consistent with the normal APP expression pattern previously reported at this stage (Bergstrom et al., 2016). Transcript abundance decreased significantly during EB formation and increased to the highest levels in 10-day old neuronally differentiated cultures. The relative ratio of edit specific Ab mRNA for stem cells, embryoid bodies and differentiated neurons was -20:1:100. Ab protein levels would likely be possibly ˜5-fold greater in differentiated neurons relative to undifferentiated stem cells.

APP expression in 10-day old differentiated neurons was measured using forward and reverse primers that span different adjacent exons along the length of the normal neuronal APP transcript (FIG. 2B). Different exon spanning primer pairs detected APP transcripts over an approximately -8-fold range, but the pattern was similar for all 3 genotypes. The average relative APP expression for all 3 primer pairs compared to H9 was 0.71 for Aβ40 and 0.5 for Aβ42, consistent with expected inactivation of only the edited APP allele confirming that editing did not drastically affect APP expression from the unedited allele. Unexpectedly, however, direct Ab expression from edited alleles was ˜30-fold lower relative to APP expression (the Ab data is replotted from FIG. 2A). This could be due to weakening of a regulatory element in the first intron of APP (Shakes et al., 2012) or alternatively to negative interference of the drug selection gene present in the insertion cassette (Davis et al., 2008). Nevertheless, direct expression levels for edit specific Ab were significantly lower than APP.

### EXAMPLE 4. Aβ PROTEIN ANALYSIS

Aβ protein could not be measured in either immunoprecipitated culture supernatants (10 ml of immunoprecipitated sample pooled from 5 samples every 2 days from a single well of a 12 well culture plate), or in guanidine hydrochloride or formic acid cell extracts (prepared from 2 individual 12 well cultures) using commercial ELISA assay kits (Invitrogen, Aβ40 #KHB3481, sensitivity 6 μg/ml; Aβ42 #KHB3441, sensitivity=10 μg/ml). Other AD-related human iPSC models were also unable to detect Aβ accumulation in cell extracts using ELISA assay, possibly indicating a technical limitation of commercial ELSISA assays (Israel et al., 2012; Muratore et al., 2014). Likely Aβ protein levels were below the detection level of the assay since all positive controls were consistent with the manufacturers reported sensitivity. These negative results are consistent with the qRT-PCR analysis and suggest that Ab levels in the directly expressing cultures are significantly lower than those generated by amyloidogenic APP processing in differentiated neuronal culture models derived from human FAD iPS cells or cells transduced with FAD genes (Choi et al., 2014; Israel and Goldstein, 2011; Muratore et al., 2014).

### EXAMPLE 5. NEURONAL DIFFERENTIATION

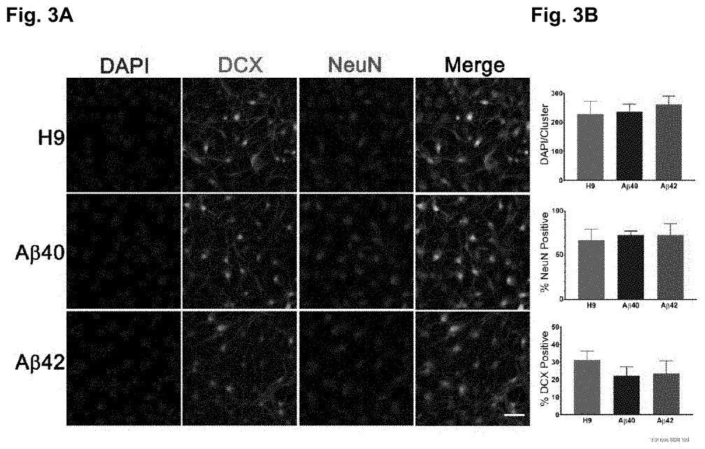

Following a differentiation protocol designed to favor production of cholinergic motor neurons, Aβ modified cells differentiated normally when compared to each other indicating that the editing process has minimal effect on normal neuronal differentiation form stem cells (Amoroso et al. 2013). The Aβ₁₋₄₂ or Aβ₁₋₄₀ modified cells generated the same number of neuronal nuclei (NeuN staining) and similar elaboration of neuronal processes (DCX staining) with no differences when either are compared to unmodified H9 cells at 10 days post-EB dissociation (FIGS. 3A-B). Marker genes characteristic of transition from stem cells (Oct4) to committed cells (Nestin) were examined in EBs. The number and size of EBs was also similar for all 3 genotypes. No significant differences among modified and unmodified lines were observed. Additionally, immature and mature neuronal markers were quantitatively similar among H9, Aβ₁₋₄₂ and Aβ₁₋₄₀ lines (Tuj1, NeuN, DCX) at early stages suggesting that neuronal development was normal.

Motor neurons are not primarily affected in AD. Basal forebrain cholinergic neurons are a cell type especially vulnerable in early stages of AD. As such, a third differentiation method designed to produce neurons with characteristics of basal forebrain cholinergic neurons may be used (see, e.g., Wicklund et al. 2010). Additionally, an astrocyte-specific differentiation protocol may be used (see, e.g., Shaltouki et al. 2013).

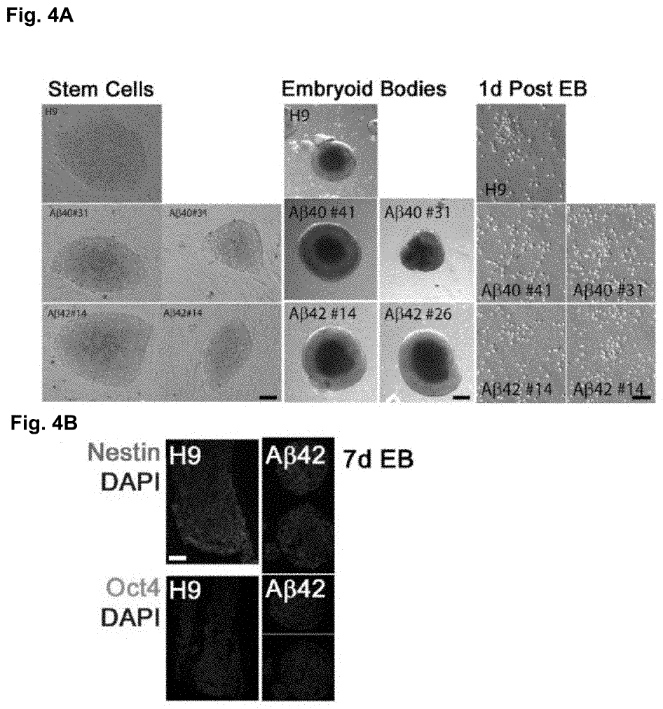

AD is a chronic and progressive neurodegenerative disease that only appears later in life. No consistent genotype specific differences in morphology of ES stage culture, embryoid body (EB) formation or the earliest stages of culture in neuronal differentiation medium were observed (FIGS. 4A-B). Additionally, earlier stage embryoid bodies (7 day old) lost their initial positive staining for OCT4 (stem cell marker) and acquired Nestin staining (early neural differentiation marker) at a similar time independent of editing. The appearance of differentiation markers in 10-day old cultures is shown in FIGS. 3A-B. The total cell number (DAPI), DCX positive cells (doublecortin, early stage neuronal differentiation) and NeuN positive cells were not significantly different among the three genotypes (ANOVA, Dunnett correction). Therefore, genomic editing and APP heterozygosity did not appear to affect neurogenesis or early neural development in the cultures and that the majority of cells (60-70%) could be classified as neurons after 10 days of differentiation. Hereafter, all culture ages for differentiated cells were specified relative to EB dissociation and plating taken as day 0.

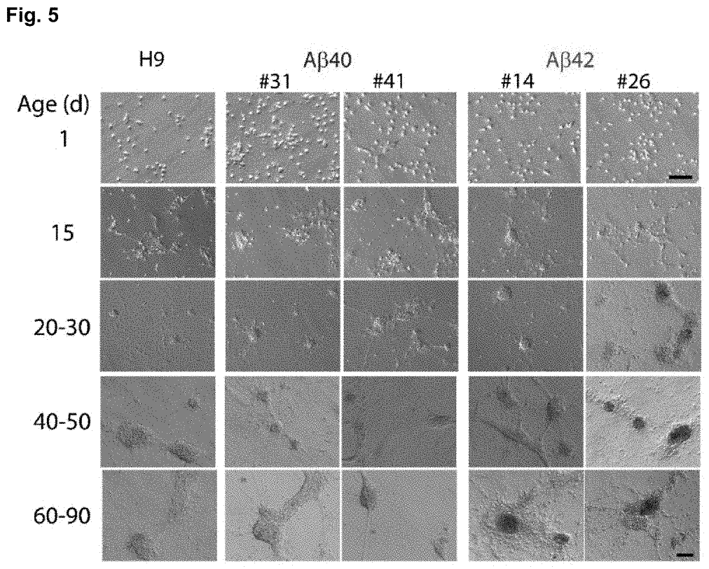

Consistent with the neuronal maker data the morphological appearance of all three genotypes, as well as the independently edited clones, remained quite similar up to about 30-days of culture (FIG. 5). One day old cultures had only isolated cells, a few of which appeared to exhibit short processes. By ˜15 days, cells appeared to self-organize into loosely defined neural clusters (NC) and elaborate neural processes, some connecting to adjacent clusters. The size of the NCs increased slightly between 20 and 30 days and began to appear more three dimensional. Many NCs were connected to each other by neural processes at this stage. The size of NCs in both edited genotypes often appeared slightly larger compared to H9 cultures, but this was not statistically significant (ANOVA, Dunnett corrected) and absent by 40 days.

At culture times of 40-50 days, Aβ42 NCs usually had a more granular appearance and were darker than the other genotypes. In one case this morphologic change was observed as early as 30 day (see FIG. 5, Aβ42 clone #26). This morphologic appearance was more prominent in Aβ42 NCs older than 60 days and thus appeared to specific to the Aβ42 edited cells. The neuronal soma for both edited genotypes lost firm attachment to the culture substrate after ˜60-70 days but remained loosely tethered to the culture dish through their neural processes. This could be easily observed when gently moving the culture dish and was not seen in the unedited H9 cultures. Culturing viable cells was unsuccessful for either edited genotype for any time longer than 120 days. In contrast, unedited H9 cultures could be maintained for >266 days.

Direct Aβ expression in edited cell lines thus decreased the survival time of neurons and resulted in specific morphologic changes, especially apparent at earlier culture times for Aβ42 edits. The absolute size of NCs had considerable variation in independent differentiations but this was a property of all 3 genotypes. These morphologic descriptions were generalized from observations made by 3 different investigators on 15 independent differentiations over a period of >2 years using several different lots of media and supplements and 2 independently isolated clones for each edited genotype.

### EXAMPLE 6. FUNCTIONAL ANALYSIS OF MODIFIED EMBRYONIC STEM CELLS EXPRESSING AN AMYLOID BETA SECRETORY PEPTIDE

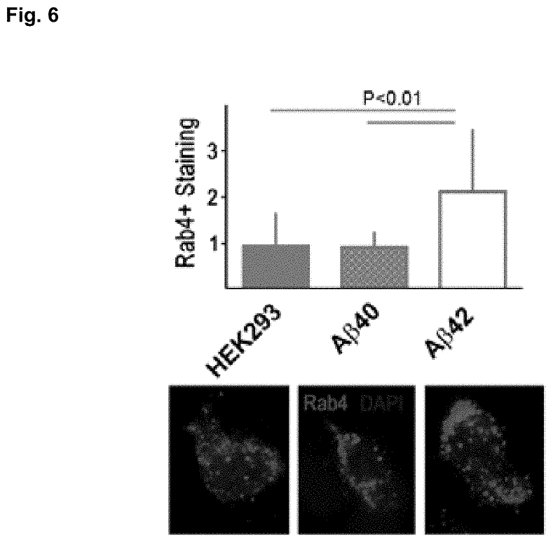

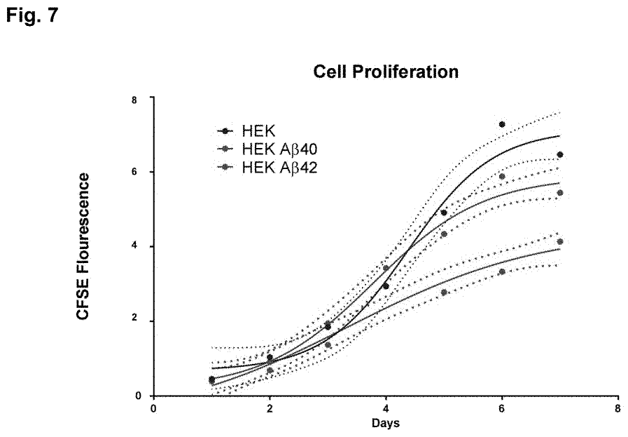

All editing strategies were initially tested in HEK293 cells. Markers of autophagy-endosomal-lysosomal (AEL) dysfunction were surveyed using qRT-PCR and immunostaining. The AEL pathway appeared to be dysfunctional specifically in Aβ₁₋₄₂ modified cells. An example difference in Rab4+staining (an endosomal marker) is shown in FIG. 6. Both the number of vesicles and size (not shown) increase in Aβ₁₋₄₂ cells. Rab4+vesicles accumulate in the Drosophila AD model and in the AD brain. Similar results were obtained for Lamp1 (lysosomal) and LC3 (autophagy) staining. Staining of several AEL markers in differentiated neurons showed similar results. Accordingly, these cells can be used to probe for underlying pathology preceding ND. While AEL function is common to all cell types, neurons have several prominent differences relative to other cell types. These differences include polarization, process elaboration, and sharing of many AEL genes with neuron-specific processes (e.g. axonal transport and synaptic transmission). Additionally, the model HEK and edited HEK cell lines showed an Aβ₁₋₄₂ specific decrease in viability (FIG. 7). Similar results were obtained for edited ES cells.

### EXAMPLE 7. CONSTRUCTION OF MODIFIED EMBRYONIC STEM CELLS WITH MODIFIED APOE GENOTYPES

Parental H9 cells are heterozygous for the APOE3/E4 (unpublished observation). These heterozygous APOE3/E4 cells may be modified using the CRISPR/Cas9 approach with targeting guide RNAs capable of modifying the APOE allele. Table 14 summarizes selected examples of APOE modifications and Aβ genotype combinations useful in embodiments described herein. Fluorescent neuronal and astrocytic lineage markers or microglial lineage markers that facilitate FACS sorting of differentiated neurons, astrocytes or microglia for co-culture analyses may also be introduced in selected cell lines. Lineage marker editing can be performed on existing cell lines or applied to APOE modified lines.

TABLE 14
APOE Modified Genotype Possibilities

| Parental Cell | APOE Edit |
|---|---|
| Aβ₁₋₄₂ modified H9 | None (E3/E4) |
| Aβ₁₋₄₀ modified H9 | None (E3/E4) |
| Unmodified H9 | None (E3/E4) |
| Aβ₁₋₄₂ modified H9 | E3→E4/E4 |
| Aβ₁₋₄₀ modified H9 | E3→E4/E4 |
| Unmodified H9 | E3→E4/E4 |
| Aβ₁₋₄₂ modified H9 | E4→E3/E3 |
| Aβ₁₋₄₀ modified H9 | E4→E3/E3 |
| Unmodified H9 | E4→E3/E3 |
| Aβ₁₋₄₂ modified H9 | E3/E4→E2/E2 |
| Aβ₁₋₄₀ modified H9 | E3/E4→E2/E2 |
| Unmodified H9 | E3/E4→E2/E2 |

### EXAMPLE 8. CONSTRUCTION OF MODIFIED EMBRYONIC STEM CELLS WITH MARKERS

Current neural differentiation strategies applied to pluripotent stem cells result in heterogeneous mixtures of cell types. To address this, a novel strategy to genetically label embryonic stem cells with lineage specific fluorescent reporters has been devised (FIG. 8). In this strategy, CRISPR/Cas9 is used to target genomic sites near the stop codon of either the GFAP, NeuN or HLA-DRA loci, marker genes that can report the astrocyte, neuronal or microglial lineage, respectively. Donor templates are constructed having an endogenous gene homology flanking a cassette containing a 2A fluorescent reporter fusion and a neoR selection gene. This method places reporter expression under direct transcriptional control of the full GFAP, NeuN or HLA-DRA promoter and is thus lineage-specific. The in-frame insertion of the 2A sequence allows self-cleavage of the reporter protein upon translation (i.e., no disruption of normal endogenous gene expression). Next, H9 cells are nucleofected with CRISPR/Cas9 and donor plasmids and subsequently plated onto murine feeder layers. NeoR colonies are then isolated and screened by genomic PCR for clones derived from HDR and confirmed by sequencing.

### EXAMPLE 9. ACCUMULATION OF AGGREGATED Aβ

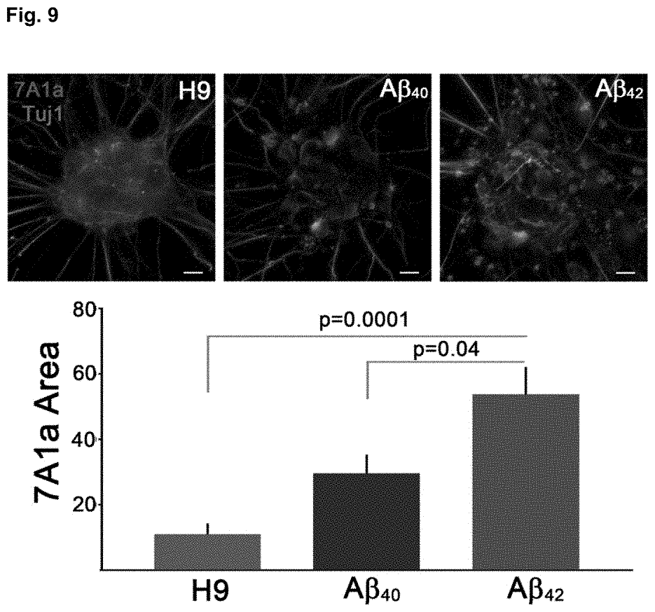

Accumulation of an aggregated form of Aβ is a characteristic feature of AD. As such, aggregation of Aβ was measured in differentiated neurons derived from unedited or edited stem cells (FIG. 9). Quantitative image analysis of immunofluorescence following staining of 32 day old (post EB) cultured neurons using an antibody specifically recognizing aggregated Aβ (7A1a) indicates significantly more accumulation in Aβ₁₋₄₂ edited neurons. The level of aggregates also appeared higher in Aβ₁₋₄₂ edited cells relative to unedited H9 but was not statistically significant (FIG. 9). These results are consistent with Aβ₁₋₄₂ having a more significant proteotoxicity than Aβ₁₋₄₀.

The main objectives of this study were to document putative AD-related phenotypes resulting from direct Aβ expression in human neurons and to compare the rate or extent of phenotypic differences between Aβ40 and Aβ42. The most commonly observed AD-related phenotypes present in most animal models as well as several iPS culture models was the accumulation of aggregated Aβ3 produced by amyloidogenic APP proteolysis (see Mungenast et al., 2016; Sasaguri et al., 2017 for reviews).

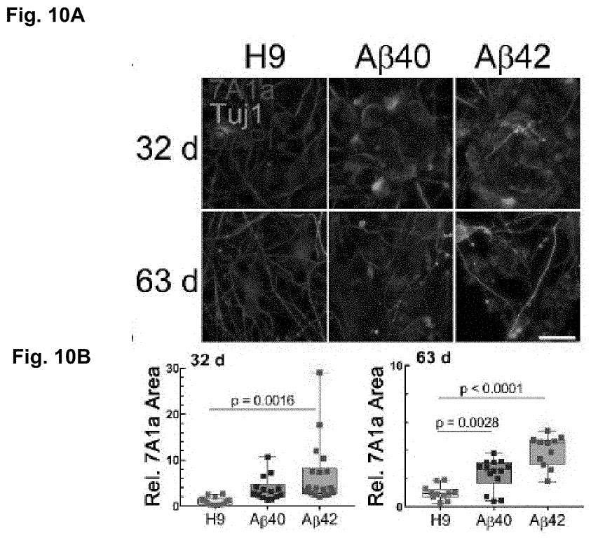

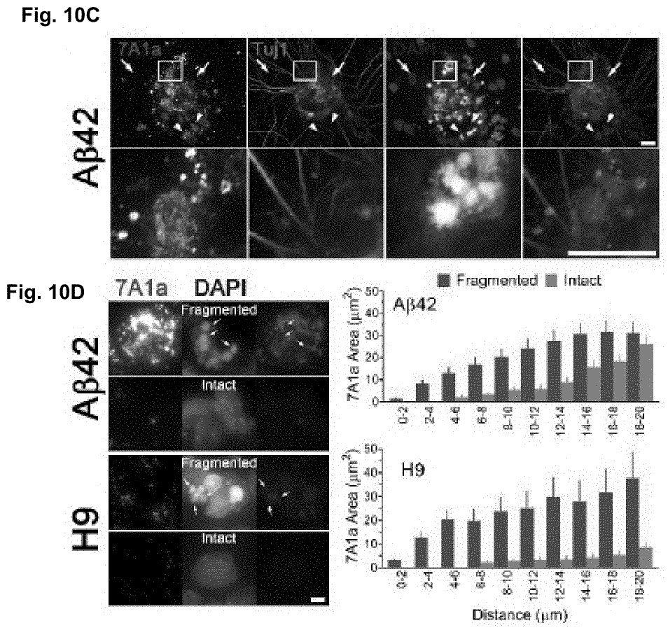

The cultures were double stained with anti-A antibody (7A1a) which specifically recognizes low and high molecular weight aggregates/oligomers of Aβ40 or Aβ42 (Ling et al., 2014; van Helmond et al., 2010) and anti-Tuj1 (TUBB3 gene product) to confirm neuronal cellular identity. In 32-day old cultures the level of 7A1a positive staining was genotype specific (FIG. 10A). The relative area of 7A1a staining (normalized to Tuj1) was minimal in H9, intermediate in Aβ40 and significantly higher in Aβ42 cultures. Compared to unedited H9 cultures, the area of 7A1a staining was -2 fold higher in Aβ40 cultures (but not statistically different from H9) and ˜3 fold higher in Aβ42 cultures (p<0.0016) at 32 days (FIG. 10B). At a later culture age (63 d) the average accumulation of 7A1a positive staining relative to H9 increased to ˜3 fold in Aβ40 and ˜4.5 fold in Aβ42 cultures. Accumulation of aggregated/oligomeric Aβ was thus progressive and faster for Aβ42 relative to Aβ40 cultures. This result was consistent with the biophysical aggregation properties of these 2 peptides in vitro (Bharadwaj et al., 2009) and the fact that both edited genes were expressed at comparable levels suggested that Aβ42 may be removed at a slower rate. Both edited genotypes had less Tuj1 positive staining which was especially evident in older Aβ cultures but not in older H9 cultures (FIG. 10A).

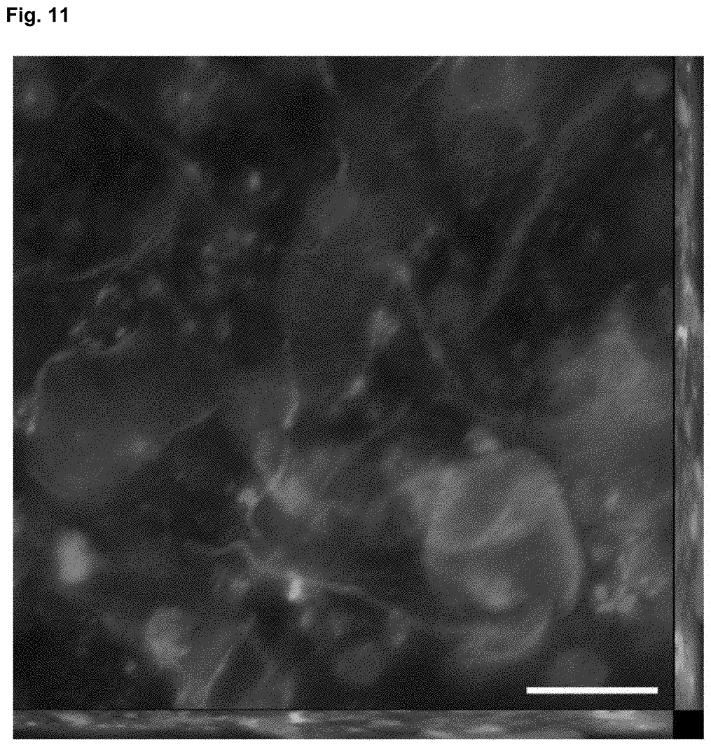

It was confirmed that 7A1a staining in Aβ edited neurons was primarily intracellular by constructing maximum intensity orthogonal projections of image stacks (FIG. 11). The staining appeared to be localized near pyknotic nuclei, a characteristic of dead or dying cells (i.e. nuclear condensation and fragmentation). Normal neuronal nuclei were large and only weakly stained with DAPI while pyknotic bodies were smaller and had significantly more intense DAPI fluorescence. FIG. 10C shows this spatial relationship in a 32-day old Aβ42 culture. Larger areas of 7A1a staining were generally absent in areas near normal nuclei but common near pyknotic nuclei. Whenever 7A1a staining was occasionally present close to normal nuclei the staining appeared to be punctate (possibly vesicular).

Also, a few cells in unedited H9 cultures with 7A1a positive staining seemed to be near pyknotic nuclei (FIG. 10D, left panel). This spatial relationship was tested by placing a counting grid of concentric circles (radius increased in 2 μm increments) over the center of mass for normal pyknotic bodies in H9 and Aβ42 cultures. The area of 7A1a staining in each ring relative to the distance from the center of mass was plotted as a histogram in FIG. 10D (right panel). Pyknotic nuclei had more 7A1a staining nearby relative to normal intact nuclei. Surprisingly, this spatial relationship was similar for both Aβ42 edited and unedited H9 neurons. This result suggests that pyknosis could be caused by aggregated/oligomeric Aβ3 derived from either direct expression or through APP amyloidogenic processing.

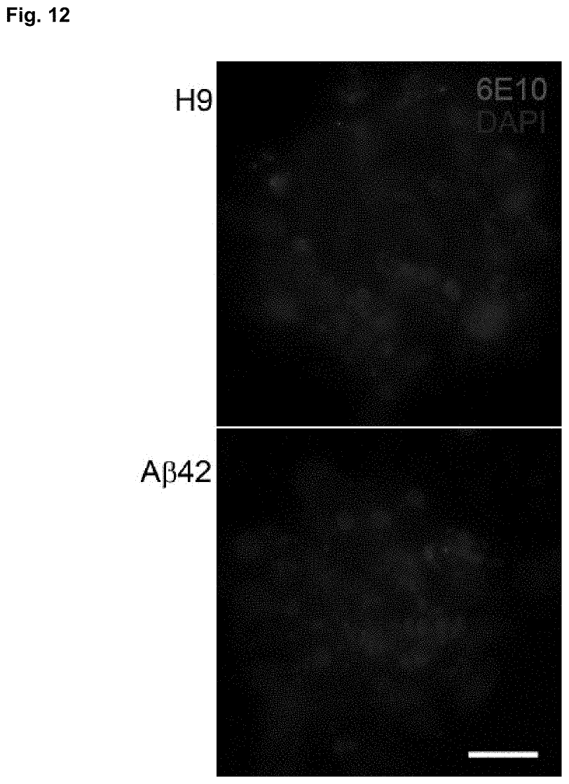

When cultures were stained using a widely used antibody that recognizes primarily monomeric Aβ3 (6E19), intracellular staining was diffuse and weak, however, staining was more prominent for H9 cells relative to Aβ3 edited cells (FIG. 12). The method of Aβ3 production may thus affect its eventual localization.

### EXAMPLE 10. SYNAPTIC DENSITY

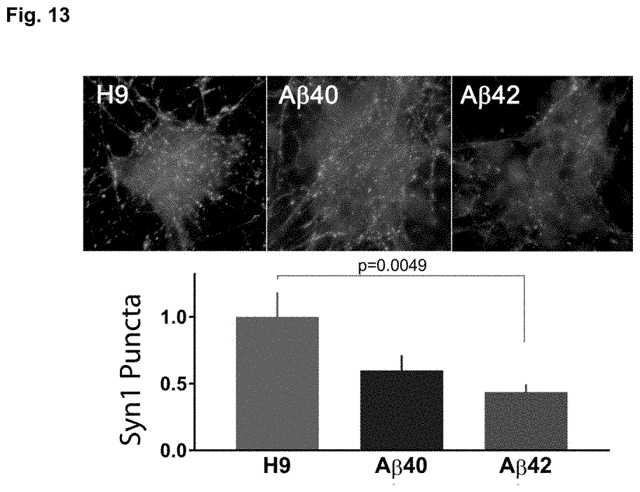

Synapse loss is another strong clinical correlate of AD and thought to precede overt ND. Synapse number was examined by staining 34-day old differentiated neurons with an antibody to synapsin 1, a maker of synaptic regions in nervous system. At this stage a significant decrease in the synapsin 1 staining specific to Aβ₁₋₄₂ edited cells was observed, as shown in FIG. 13. Since other measures of development appear normal up to this age, this may indicate Aβ₁₋₄₂ dependent synaptic loss and thus be useful for screening agents that may prevent this early AD feature.

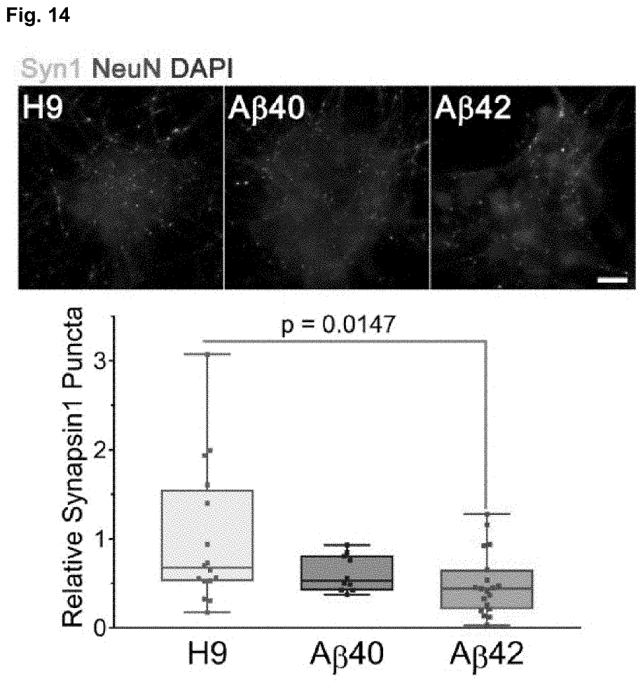

A decrement in the number of synapses is a consistent and early AD phenotype that correlates well with cognitive decline, even during preclinical disease stages (Forner et al., 2017). Several transgenic mouse models exhibited synaptic deficits, however this phenotype was not described in human AD cell culture models. 34-day old cultures were stained with anti-synapsin 1 antibody (a presynaptic marker) to estimate the number of synapses present in neuronal clusters from the different genotypes. As shown in FIG. 14, all 3 genotypes at this culture stage had a significant number of synapsin positive puncta. There are, however, ˜50% fewer synapsin positive puncta in Aβ42 edited samples (p <0.0147) relative to unedited H9 samples. Aβ40 samples had ˜20% fewer synapsin puncta but did not reach significance. There was thus a graded genotype dependent difference in the number of synapsin puncta at this culture stage: H9>Aβ40>>Aβ42. The results establish that synaptic number was reduced to a greater extent in Aβ42 compared to Aβ40 cultures, a result that was consistent with the concept that Aβ negatively affects synaptic capacity (Forner et al., 2017).

### EXAMPLE 11. NEURODEGENERATION

Viability assays are a preferred way to establish the quantitative rate of ND. This is an important parameter for particular combinations co-cultured cells with defined ApoE astrocyte/neuronal genotypes as well as mixtures of various cell types and the action of agents to prevent or slow ND.

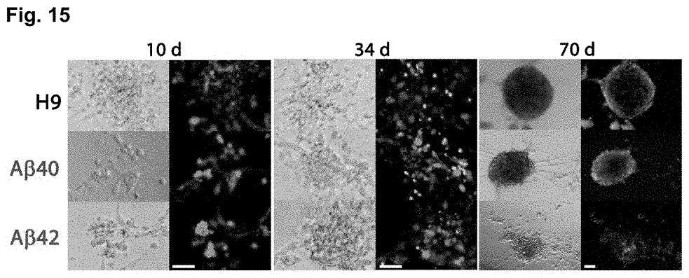

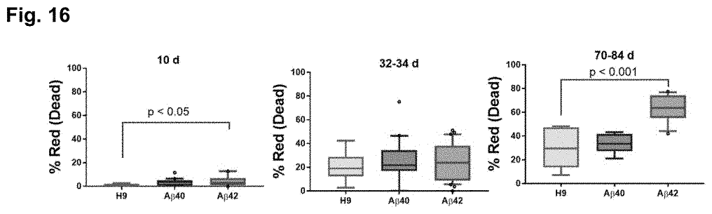

A live-dead (calcein AM/ethidium homodimer) fluorescence assay performed on various stages of differentiated neurons (up to 200 days following EB dissociation) was used. Results established that the Aβ₁₋₄₂ edited neurons were specifically susceptible to neuronal cell death relative to either Aβ₁₋₄₀ or unedited cells, as shown in FIGS. 15-16. This suggests that Aβ₁₋₄₂ edited cells could be useful to screen for agents that prevent or slow ND.

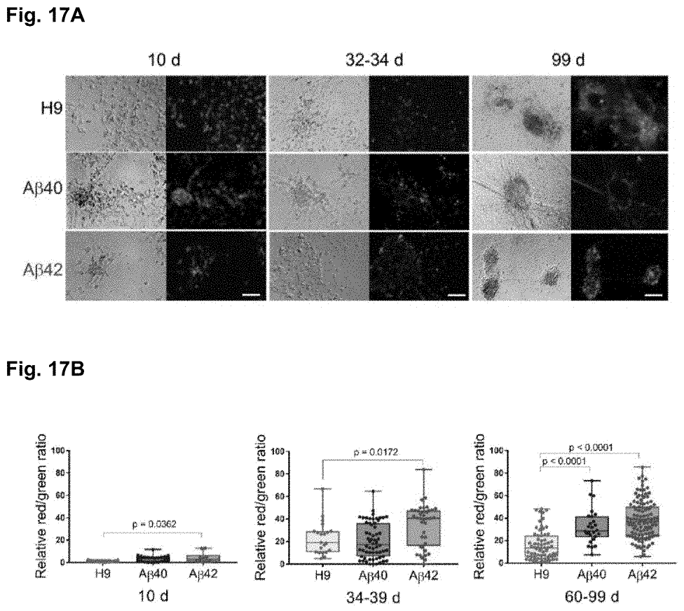

AD is a chronic progressive disease with end stage neuronal cell death, a phenotype that has been particularly difficult to document in most current experimental models. A fluorescent live/dead assay was used to assess neuronal viability at 3 different culture ages. Representative morphological and fluorescent images of the same field are shown in FIG. 17A. Despite a normal morphologic appearance and similar numbers of neurons in 10-day old cultures, a slightly higher proportion of ethidium homodimer fluorescence (dead cells) was found in Aβ42 cultures even at this early culture stage (FIG. 17B). At an intermediate culture age (34-39 days) when Aβ42 neuronal clusters had significantly fewer synapsin puncta, the relative ethidium homodimer fluorescence was greater in Aβ42 compared to either Aβ40 or H9 cultures. When maintained for longer times (i.e. >-60 days) both Aβ40 and Aβ42 edited cultures exhibit significantly more relative ethidium homodimer fluorescence compared to unedited H9 cultures. Since most cells under our culture conditions are positively identified as neurons (˜70-90% Tuj1 positive), the editing resulted in progressive ND. This phenotype appeared at a faster rate for Aβ42 cells relative to Aβ40 cells and was dependent on editing. No viable cells remained in edited culture older than 120 days while H9 cultures still appeared healthy even after 266 days. This edit specific progressive ND also appeared to be chronic because of the extended time necessary for its elaboration.

### EXAMPLE 12. ENDOLYSOSOMAL PATHWAY PHENOTYPES

Dysfunction of the endolysosomal pathway plays an important role in several neurodegenerative diseases, including AD (Nixon, 2017). Pathway dysfunction is a consistent feature of several animal and cellular AD models (Israel et al., 2012; Nilsson et al., 2013) as well as an early phenotype in AD (Cataldo et al., 2004) and can be inferred by accumulation of an abnormal number or size of characteristic vesicles.

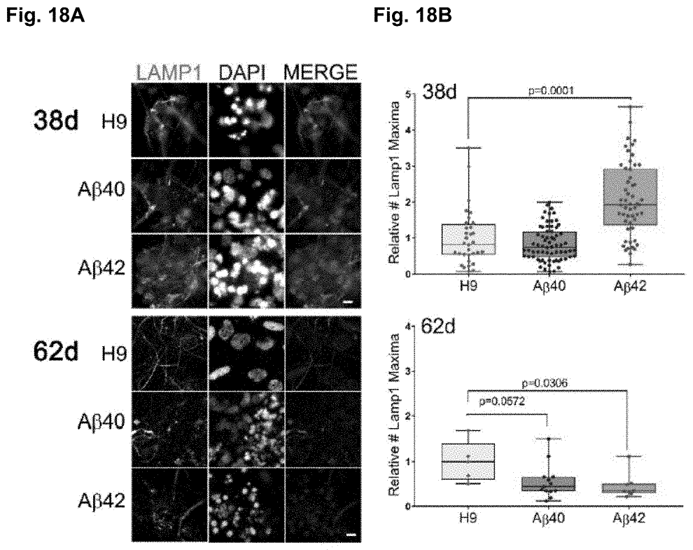

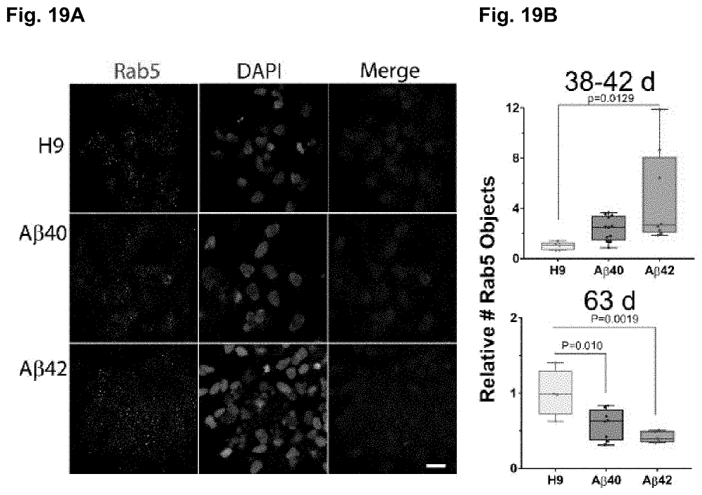

Using vesicle type specific antibody staining, the relative number of punctate vesicular structures was counted in neurons. FIGS. 18A-B presents representative images and quantitative analysis for 38- and 62-day old cultures stained with anti-lysosomal associated membrane protein 1 (LAMP1) antibody. There was a -2-fold increase in LAMP1 positive puncta in 38-day old Aβ42 cultures relative to either Aβ40 or unedited H9 cultures. This finding agrees with the reduced neuronal viability in Aβ42 samples and synapsin1 puncta at this culture stage. In older cultures (62 days) the number of Lamp1 puncta relative to unedited H9 cells was decreased ˜60% in Aβ42 and ˜50% in Aβ40 samples (although not significant). This decrease thus correlates with ND present in both edited genotypes in older cultures. Abnormal accumulation of lysosomal related vesicles may thus be a consequence of direct Aβ expression in human neurons. The number of Rab5 stained puncta, a marker for early endosomes necessary for vesicular maturation leading to lysosomal fusion (Poteryaev et al., 2010) is shown in FIGS. 19A-B. The pattern was similar to LAMP1 puncta. There was a significant increase in Rab5 puncta in 38-42-day old Aβ42 relative to H9 samples and a non-significant increase in Aβ40 samples. Both edited genotypes also showed a significant decrease in Rab5 puncta in older 63-day cultures.

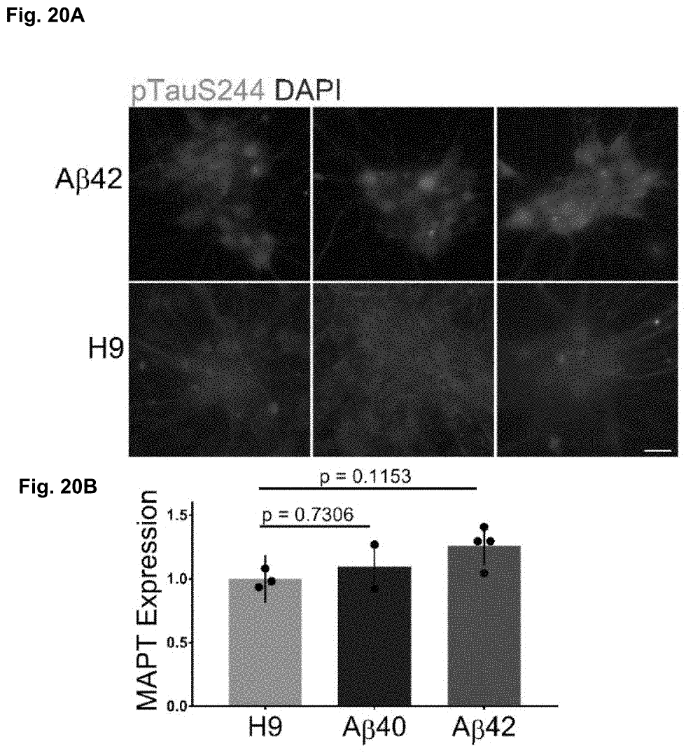

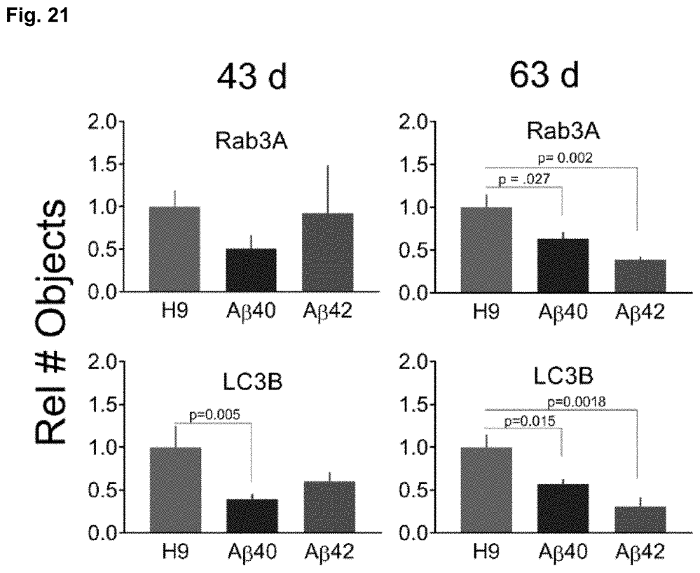

62-day old Aβ42 edited cultures had a similar staining intensity not significantly different from unedited cultures. Only a single anti-phospho tau specific antibody (targeting serine 244) (Grueninger et al., 2010) was used. The distribution of phospho-tau staining did appear more somal in edited cells, but this was likely due to a prominent reduction in neurites (recognized by Tuj1 staining) at this late culture age. Increased tau phosphorylation is unlikely to be a prominent phenotype of direct Aβ42 expression. Additionally, RNAseq data for MAPT (the gene encoding tau) expression was similar for all 3 genotypes. These results are shown in FIGS. 20A-B. One possible explanation for this absence of increased phospho-tau in edited cells is that the caudal neurons are reportedly less susceptible to increased phospho-tau compared to more rostral neurons (Muratore et al., 2017).

Object counts were also obtained for Rab3A staining, a synaptic vesicular gene important for regulating normal synaptic neurotransmission (Schluter, 2004) and LC3B staining, an autophagosome vesicle marker necessary for delivering mature autophagic/endosomal vesicles to lysosomes for cargo digestion which is associated with AD (Boland et al., 2008). The number of Rab3A puncta was not significantly different among any of the genotypes in 43-day old cultures but both genotypes exhibit a reduction in 63-day old cultures. Both Aβ40 and Aβ42 samples had a reduction in LC3B puncta in 43-day old cultures (only Aβ40 was significant) as well as in 63-day cultures. Results were more variable for all three genotypes and counts are presented in FIG. 21.

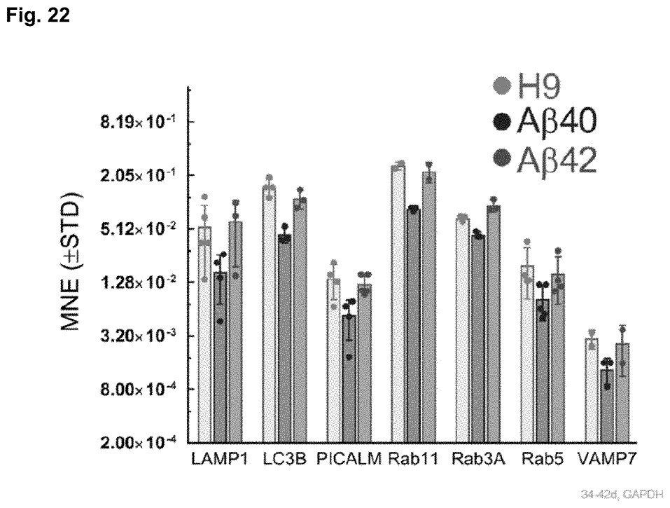

These results indicate that endolysosomal pathway dysfunction was associated with Aβ edited samples and that Aβ42 samples appeared to be affected at earlier times and to a greater extent than Aβ40 samples. These changes were not likely attributable to changes in gene expression for key vesicular genes since qRT-PCR analysis did not find any genotype specific expression differences (FIG. 22). Since Aβ was directly expressed in edited cultures, these potential AD-related phenotypes are likely to be independent of APP amyloidogenic processing believed to occur in large part within endolysosomal vesicles (Haass et al., 2012).

### EXAMPLE 13. AO-DEPENDENT DIFFERENTIAL GENE EXPRESSION

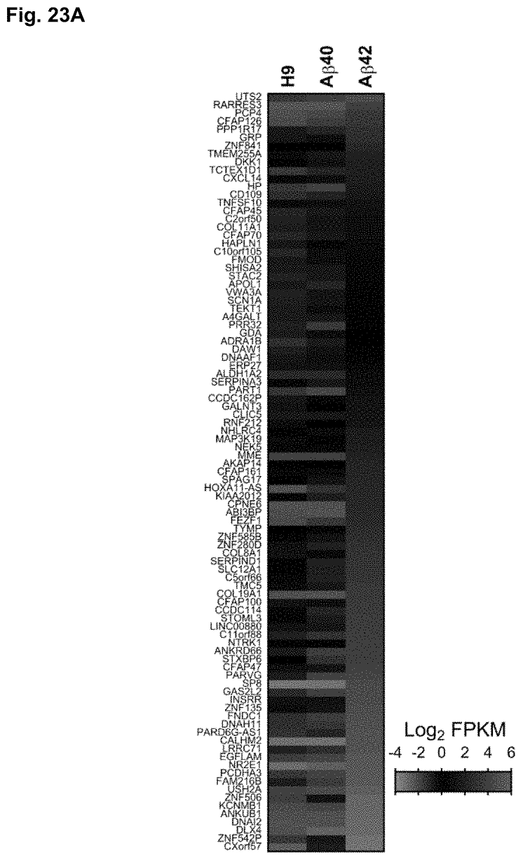

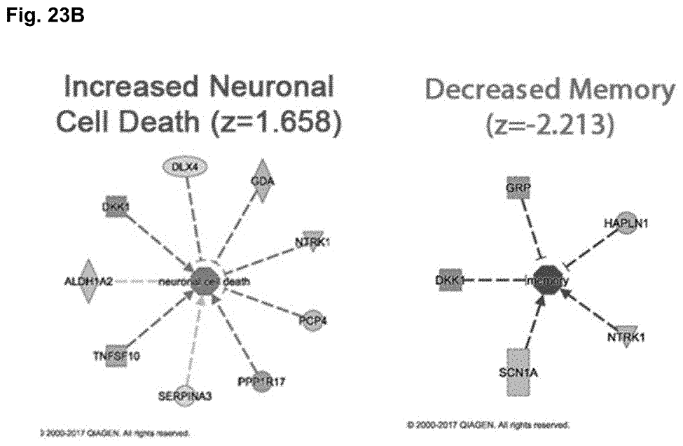

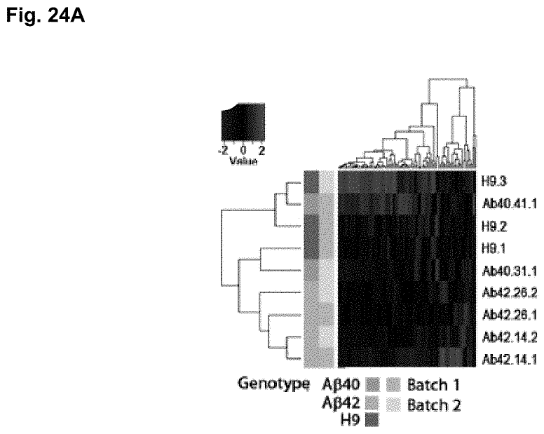

Changes in gene expression allow for the testing of mechanisms of Aβ₁₋₄₂ dependent ND that were observed. A whole transcriptome RNAseq analysis of several biological replicates of all 3 genotypes for 34-day old (post-EB) differentiated neurons was completed. Cluster analysis of the RNAseq data reveals that Aβ₁₋₄₂ cells differentially expressed several genes relative to Aβ₁₋₄₀ or unedited H9 cells (absolute value of fold change >1.5, FDR<0.05). A heat map indicating of up or down regulated genes meeting these criteria is shown in FIGS. 23A-B. When this set of genes was submitted to Ingenuity Pathway Analysis, remarkably the highest scoring pathway function was increased neuronal cell death and the lowest scoring pathway was decreased memory. Both of these are obvious phenotypes of AD suggesting more than coincidental relevance for the disclosed cell lines and reinforcing their potential usefulness to screen for new agents to treat AD. Pathway analysis additionally identified one Canonical Pathway: Neuroprotective Role of Thop1 in Alzheimer's for the Ab42 vs H9 comparison. The genes affected in Aβ₁₋₄₂ relative to H9 were: MME (Neprylisin) and SERPINA3 (ACT), p=9.95 E-03, Overlap =5% (2/40).

The edited cell lines present a particularly favorable opportunity for whole transcriptome RNA-Seq analysis to identify differentially expressed genes (DEGs) that may be mechanistically linked to Aβ-dependent ND. They were not confounded by uncontrolled amyloidogenic APP proteolysis, overexpression of non-Aβ fragments and were near isogenic. RNA-Seq expression was performed using mRNA isolated from 36-38-day old cultures. This is a stage where phenotypes are either exclusive (i.e. reduced number of synapses, reduced neuronal viability and increased accumulation of lysosomes and endosomes) or more penetrant (greater accumulation of aggregated Aβ) for the Aβ42 editing compared to Aβ40 editing. RNA isolated from 3 independent H9 culture samples served as the reference control to identify DEGs for each edited genotype. All three genotypes are heterozygous for the major sporadic AD risk allele (i.e. ϵ4/ϵ3) and thus in an appropriate human genetic context relevant to a large proportion of SAD cases (Corder et al., 2008).

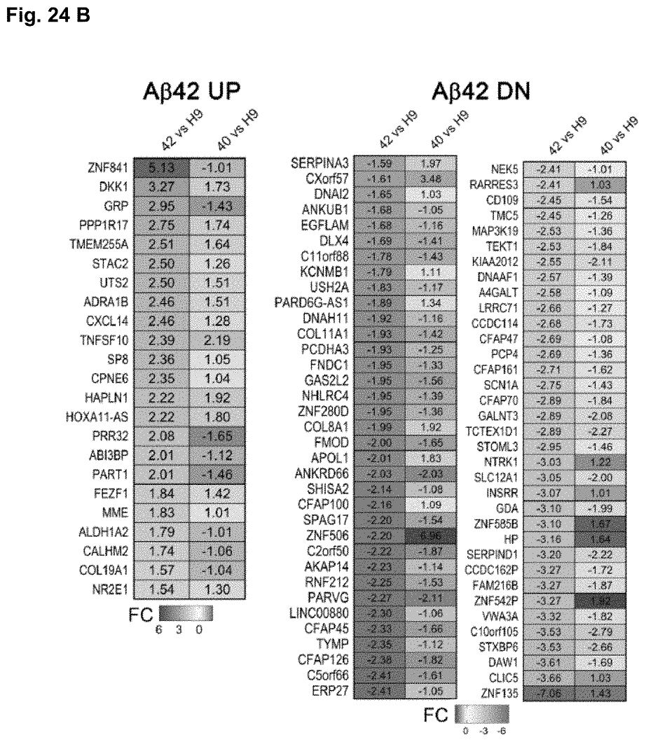

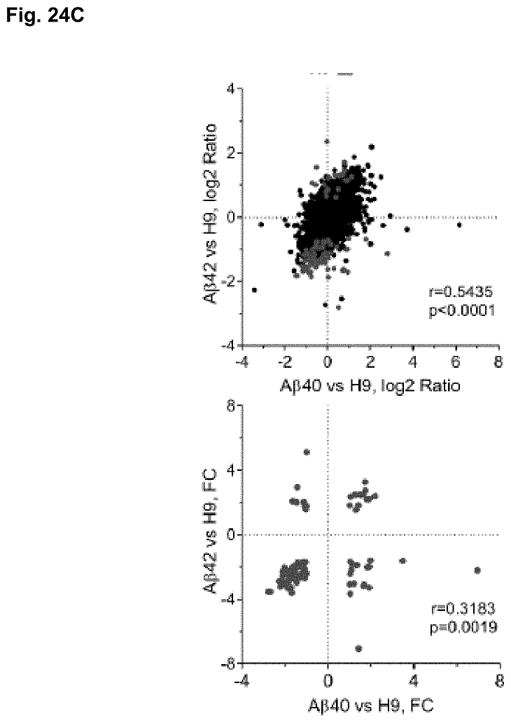

Differential expression was tested for 18,259 genes (i.e. genes that had an FPKM >0.1 in 50% of samples). Results of hierarchical clustering along with an expression heat-map for the batch centered sample medians of individual samples are shown in FIG. 24A. The 4 Aβ42 samples clustered together on the same branch of the dendrogram. One Aβ40 sample (#31.1) clustered adjacent to the Aβ42 group while the other (#41.1) appeared more like unedited H9 samples indicating that whole transcriptome expression was more similar among individual Aβ42 edited samples relative to either Aβ40 or unedited H9 samples which agreed with phenotypic penetrance at this culture age. DEGs may thus be mechanistically associated with Aβ42-dependent affected pathways related to these phenotypes.

DEGs were defined by first identifying genes that vary between Aβ42 vs H9 and then filtering genes with a similar directional change for Aβ40 that using a more liberal criteria (to avoid keeping genes marginally not significant in the Aβ40 vs H9 comparison). All 93 DEGs for the Aβ42 vs H9 comparison are shown in FIG. 24B as a fold change (FC) heat map. There were 23 UP and 70 DN (down) regulated genes which were used for functional/annotation enrichment analysis. This number is rather small compared to numerous other AD related studies of DEGs in patient samples or even iPS cell lines where thousands of DEGs are often identified (Annese et al., 2018; Blalock et al., 2004; Israel et al., 2012; Zhang et al., 2013). The directional FC (fold-change) was similar for most genes in the Aβ40 samples compared to Aβ42. The Pearson correlation coefficients for log2 ratios of all Aβ42 vs H9 compared to Aβ40 vs H9 genes was 0.5434 (all genes, linear-regression p-value <0.0001, FIG. 24C, top), and the correlation coefficient for the DEG FC values was 0.3183 (differentially expressed genes, linear regression p-value=0.0019, FIG. 24C, bottom). Aβ dependent changes in gene expression thus appeared similar for Aβ42 and Aβ40 samples.

GO enrichment analysis of the UP and DN regulated genes for the Aβ42 vs H9 comparison did not identify functional enrichment for UP genes after correcting for FDR. The statistical power of this approach, however, is likely limited when using a small number (23) of input DEGs. For DN genes, however, 13 out of 70 (19%) were related to cilia functions and were significantly overrepresented (i.e. FDR<0.05) (CCDC114, CFAP100, CFAP126, CFAP45, CFAP70, DAW1, DNAAF1, DNAH11, DNAI2, SPAG17, STOML3, TEKT1, USH2A). Interestingly, 5 of these “cilia” genes (DAW1 DNAH11 DNAI2 GDA TEKT1) were also differentially expressed in a hippocampal AD vs non-AD RNA-Seq study (Annese et al., 2018) suggesting that cilia related pathways may also be affected in AD. Using unadjusted p-values, microtubule and cytoskeletal genes were also over represented (CCDC114, CFAP100, CFAP126, CLIC5, DNAAF1, DNAH11, DNAI2, GAS2L2, PARVG, SPAG17, TEKT1, USH2A) as well as genes associated with vesicle lumen (COL11A1, COL8A1, ERP27). Overrepresented molecular functions included neurotrophin receptor associated terms (NTRK1) and peptidase regulatory roles (CD109, SERPINA3, SERPIND1).

GATHER (changlab.uth.tmc.edu/gather/gather.py) was used to broaden the search for relationships/pathways in the Aβ42 DEGs. Two GO terms were statistically significant for UP genes (FDR<0.05): GO:0007267: cell-cell signaling (ADRA1B, CPNE6, CXCL14, MME, TNFSF10, UTS2) and GO:0007154: cell communication (ADRA1B, COL19A1, CPNE6, CXCL14, DKK1, GRP, HAPLN1, MME, STAC2, TNFSF10, UTS2). DN genes included two overlapping GO terms: GO:0015698: inorganic anion transport and GO:0006820: anion transport (CLIC5 COL11A1 COL8A1 SLC12A1). KEGG pathways with an FDR<0.25 included hsa04080: Neuroactive ligand-receptor interaction (ADRA1B, GRP, UTS2) and hsa05010: Alzheimer's disease [MME] for UP genes. DN genes were hsa04512: ECM-receptor interaction (COK11A1, FNDC1).

GSEA KEGG analysis (software.broadinstitute.org/gsea/index.jsp) is an additional way to discover potential pathway relationships and is not limited by using a small list of input genes since input can be a rank order list of FC values for all detected genes. GSEA was performed using a rank ordered FC list (18,233 genes) and these were compared to all KEGG pathways. The Aβ42 vs H9 list identified 118/170 KEGG gene sets that were upregulated. Twenty-two had a nominal p value <0.05 and 3 of these had an FDR <25%. The top scoring KEGG pathway was NEUROACTIVE LIGAND RECEPTOR INTERACTION (hsa04080, Normalized Enrichment Score=2.12, p<0.01, FDR=0.014). The gene list included 79 of the 219 (36%) genes in this pathway suggesting widespread changes in neuroactive ligand receptor signaling was a consequence of direct Aβ42 expression. This can plausibly be related to the DN regulated expression of “cilia” related genes since primary cilia in neurons are believed to be a major organelle signaling hub known to express a host of neuroactive ligand receptors (Guemez-Gamboa et al., 2014). No KEGG pathways reached significance (FDR<0.05) for DN genes or for a separate analysis of ranked Aβ40 vs H9 DEG FC values.

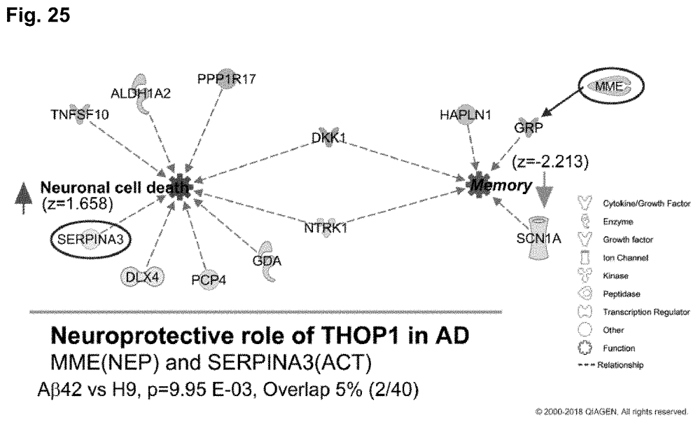

DEGs were analyzed with Ingenuity Pathway Analysis (IPA). Remarkably, for the Aβ42 vs H9 comparison, the highest and lowest z scores were obtained for the functions “Increased Neuronal Cell Death” (z=1.658) and “Decreased Memory” (z=-2.213), two biological processes with obvious relevance to AD. FIG. 25 shows the individual DEGs identified by this analysis color coded by intensity for FC values. The “Decreased Memory” and “Increased Neuronal Cell Death” pathways are connected through the overlap of DKK1 and NTRK1. IPA disease related pathways returned the “Neuroprotective Role of THOP1 in AD” as the top scoring canonical pathway (p=9.95 E-3). This pathway was also significant for a hippocampal DEG analysis of LOAD RNA-Seq data (Annese et al., 2018). Thimet oligopeptidase (product of THOP1) is reportedly neuroprotective for Aβ toxicity in cortical neurons and can degrade soluble Aβ but not aggregated Aβ42 (Pollio et al., 2008; Yamin et al., 1999). The DEGs in the Aβ42 vs H9 comparison represent only a small fraction of the 40 genes in this pathway. They were MME (aka NEP, neprylisin) and SERPINA3 (aka ACT) (indicated on the bottom of FIG. 25). MME is not directly related to decreased memory in IPA, but is included because of its potential indirect relationship through GRP (Saghatelian et al., 2004). SERPINA3 is a member gene of the “Neuronal Cell Death” category in IPA and both genes are part of the extracellular arm of the THOP1 in AD pathway in IPA. MME, is an Aβ degrading enzyme with increased expression in the Aβ42 edited cells and SERPINA3, is a serine protease inhibitor with decreased expression which co-localizes with Aβ in AD plaques (Abraham et al., 1988).

There is no general consensus regarding a “signature” set of AD related DEGs, especially those that related to early LOAD pathogenic mechanisms making it challenging to compare the expression data with patient samples that likely contain signals from many different non-neuronal cell types, co-morbidities and many complex combinations of genetic variance. Nevertheless, some potentially relevant comparisons were found. For example, the GeneCards database (www.genecards.org) has 6,672 genes identified as “Alzheimer's related genes”. This is a rather large list not restricted to DEG analysis but also including GWAS hits as well as other types of associations. For the UP genes it was found 10/23 (43%) that overlapped (TNFSF10, DKK1, GRP, CALHM2, MME, ALDH1A2, CXCL14, PPP1R17, TMEM255A, HAPLN1) and 17/70 (24%) DN genes (SHISA2, DNAH11, SERPIND1, SCN1A, APOL1, HP, ERP27, SERPINA3, STXBP6, CFAP70, PARVG, GDA, PCP4, NTRK1, TMC5, STOML3, RARRES3) suggesting some potential AD relevance. A recent RNA-Seq analysis of hippocampal tissue from SAD vs non-SAD patient samples (Annese et al., 2018) identified 2,064 DEGs. Only 3 out 23 (13%) UP genes were found overlapped (not statistically significant; Fisher's exact test, p=0.46) (HAPLN1, CPNE6, TNFSF10). In contrast, 22 out of 70 (31%) DN genes overlapped (Fisher's exact test, p=2.5×10-6) (DAW1, FAM216B, GDA, TCTEX1D1, PCP4, CCDC114, LRRC71, A4GALT, MAP3K19, TEKT1, CD109, TMC5, RARRES3, LINC00880, PARVG, ANKRD66, FNDC1, DNAH11, C11orf88, ANKUB1, DNAI2, SERPINA3). Four of these DN genes had an opposite directional FC, while others agreed with the DEGs. This significant overlap suggests that DEGs in the disclosed Aβ-dependent neuronal model may thus have relevance to the AD, including the possible involvement of cilia dysfunction as mentioned above.

### REFERENCES

The references cited in the specification above and those listed below are hereby incorporated by reference as if fully set forth herein.

Abraham, C. R., et al. (1988). Immunochemical identification of the serine protease inhibitor al -antichymotrypsin in the brain amyloid deposits of Alzheimer's disease. Cell 52, 487-501.

Abramowski, D., et al. (2012). Transgenic expression of intraneuronal Abeta42 but not Abeta40 leads to cellular Abeta lesions, degeneration, and functional impairment without typical Alzheimer's disease pathology. J Neurosci 32, 1273-1283.

Allen, M., et al. (2016). Human whole genome genotype and transcriptome data for Alzheimer's and other neurodegenerative diseases. Sci. Data 3, 160089.

Amoroso, M. W., et al. (2013). Accelerated High-Yield Generation of Limb-Innervating Motor Neurons from Human Stem Cells. J. Neurosci. 33, 574-586.

Anders, S., et al., HTSeq—a Python framework to work with high-throughput sequencing data Bioinformatics, 2015. 31(2): p. 166-169.

Annese, A., et al. (2018). Whole transcriptome profiling of Late-Onset Alzheimer's Disease patients provides insights into the molecular changes involved in the disease. Sci. Rep. 8, 4282.

Arber, C., et al. (2017). Stem cell models of Alzheimer's disease: progress and challenges. Alzheimers. Res. Ther. 9, 42.

Ashburner, M., et al., Gene Ontology: tool for the unification of biology. Nat Genet, 2000. 25(1): p. 25-29.

Ashe, K. H., et al. (2010). Probing the biology of Alzheimer's disease in mice. Neuron 66, 631-645.

Bales, K. R. (2012). The value and limitations of transgenic mouse models used in drug discovery for Alzheimer's disease: an update. Expert Opin. Drug Discov. 7, 281-97.

Benilova, I., et al. (2012). The toxic Aβ oligomer and Alzheimer's disease: an emperor in need of clothes. Nat. Neurosci. 15, 349-357.

Benjamini, Y. and Y. Hochberg, Controlling the false discovery rate: a practical and powerful approach to multiple testing. J R Stat Soc Series B Stat Methodol, 1995. 57: p. 289-300.

Berbari, N. F., et al. (2009). The Primary Cilium as a Complex Signaling Center. Curr. Biol. 19, R526-R535.

Bergström, P., et al. (2016). Amyloid precursor protein expression and processing are differentially regulated during cortical neuron differentiation. Sci. Rep. 6, 29200.

Bharadwaj, P. R., et al. (2009). Abeta aggregation and possible implications in Alzheimer's disease pathogenesis. J Cell Mol Med 13, 412-421.

Blalock, E. M., et al. (2004). Incipient Alzheimer's disease: Microarray correlation analyses reveal major transcriptional and tumor suppressor responses. Proc. Natl. Acad. Sci. 101, 2173-2178.

Boland, B., et al. (2008). Autophagy Induction and Autophagosome Clearance in Neurons: Relationship to Autophagic Pathology in Alzheimer's Disease. J. Neurosci. 28, 6926-6937.

Castillo, E., et al. (2017). Comparative profiling of cortical gene expression in Alzheimer's disease patients and mouse models demonstrates a link between amyloidosis and neuroinflammation. Sci. Rep. 7, 17762.

Cataldo, A. M., et al. (2004). Abeta localization in abnormal endosomes: association with earliest Abeta elevations in AD and Down syndrome. Neurobiol Aging 25, 1263-1272.

Cermak, T., et al. (2011). Efficient design and assembly of custom TALEN and other TAL effector-based constructs for DNA targeting. Nucleic Acids Res 39, e82.

Chang, J. T. and J. R. Nevins, GATHER: a systems approach to interpreting genomic signatures. Bioinformatics, 2006. 22(23): p. 2926-2933.

Choi, S. H., et al. (2014). A three- dimensional human neural cell culture model of Alzheimer's disease. Nature 515, 274-278.

Corder, A. E. H., et al. (2008). Gene Dose of Apolipoprotein E Type 4 Allele and the Risk of Alzheimer' s Disease in Late Onset Families Published by : American Association for the Advancement of Science Stable 261, 921-923.

Davis, R. P., et al. (2008). A protocol for removal of antibiotic resistance cassettes from human embryonic stem cells genetically modified by homologous recombination or transgenesis. Nat. Protoc. 3, 1550-1558.

De Strooper, B., and Karran, E. (2016). The Cellular Phase of Alzheimer's Disease. Cell 164, 603-615.

Engel, M., et al. (2016). Common pitfalls of stem cell differentiation: a guide to improving protocols for neurodegenerative disease models and research. Cell. Mol. Life Sci. 73, 3693-3709.

Forner, S., et al. (2017). Synaptic Impairment in Alzheimer's Disease: A Dysregulated Symphony. Trends Neurosci. 40, 347-357.

Freude, K. K., et al. (2011). Soluble amyloid precursor protein induces rapid neural differentiation of human embryonic stem cells. J. Biol. Chem. 286, 24264-74.

Genin, E., et al. (2011). APOE and Alzheimer disease: a major gene with semi-dominant inheritance. Mol. Psychiatry 16, 903-7.

Ghavami, S., et al. (2014). Autophagy and apoptosis dysfunction in neurodegenerative disorders. Prog. Neurobiol. 112, 24-49.

Grueninger, F., et al. (2010). Phosphorylation of Tau at S422 is enhanced by Aβ in TauPS2APP triple transgenic mice. Neurobiol. Dis. 37, 294-306.

Guemez-Gamboa, A., et al. (2014). Primary Cilia in the Developing and Mature Brain. Neuron 82, 511-521.

Hara, T. et al. Suppression of basal autophagy in neural cells causes neurodegenerative disease in mice. Nature 441, 885-889 (2006).

Haass, C., and Selkoe, D. J. (2007). Soluble protein oligomers in neurodegeneration: lessons from the Alzheimer's amyloid beta-peptide. Nat. Rev. Mol. Cell Biol. 8, 101-12.

Haass, C., et al. (2012). Trafficking and proteolytic processing of APP. Cold Spring Harb. Perspect. Med. 2, a006270.

Hsu, F., et al., The UCSC Known Genes. Bioinformatics, 2006. 22(9): p. 1036-1046.

Hu, X., et al. (2009). Amyloid seeds formed by cellular uptake, concentration, and aggregation of the amyloid-beta peptide. Proc. Natl. Acad. Sci. U. S. A. 106, 20324-9. Iijima, K., et al. (2004). Dissecting the pathological effects of human Abeta40 and Abeta42 in Drosophila: a potential model for Alzheimer's disease. Proc Natl Acad Sci U S A 101, 6623-6628.

Iijima-Ando, K., and Iijima, K. (2009). Transgenic Drosophila models of Alzheimer's disease and tauopathies. Brain Struct Funct 214, 245-262.

Ilie-Cosmin Stancu, et al. (2014). Models of beta- amyloid induced Tau-pathology: the long and folded road to understand the mechanism Molecular Neurodegeneration. Mol. Neurodegener. 9, 1750-1326.

Israel, M. A., and Goldstein, L. S. (2011). Capturing Alzheimer's disease genomes with induced pluripotent stem cells: prospects and challenges. Genome Med 3, 49.

Israel, M. A., et al. (2012). Probing sporadic and familial Alzheimer's disease using induced pluripotent stem cells. Nature 482, 216-220.

Karran, E., and De Strooper, B. (2016). The amyloid cascade hypothesis: are we poised for success or failure? J. Neurochem.

Kim, D., et al., TopHat2: accurate alignment of transcriptomes in the presence of insertions, deletions and gene fusions Genome Biology, 2013. 14(4): p. 1-13.

Kim, J. et al. microRNA-33 Regulates ApoE Lipidation and Amyloid- Metabolism in the Brain. J. Neurosci. 35, 14717-14726 (2015).

Kim, J., Basak, J. M., and Holtzman, D. M. (2009). The Role of Apolipoprotein E in Alzheimer's Disease. Neuron 63, 287-303.

Kim, J., et al. (2007). Abeta 40 Inhibits Amyloid Deposition In Vivo. J. Neurosci. 27, 627-633.

Kim, Y. H., et al. (2015). A 3D human neural cell culture system for modeling Alzheimer's disease. Nat. Protoc. 10, 985-1006.

Kondo, T., et al. (2013). Modeling Alzheimer's Disease with iPSCs Reveals Stress Phenotypes Associated with Intracellular Aβ and Differential Drug Responsiveness. Cell Stem Cell 12, 487-496.

LaFerla, F. M., et al. (1995). The Alzheimer's A beta peptide induces neurodegeneration and apoptotic cell death in transgenic mice. Nat Genet 9, 21-30.

Lawrence, M., et al., Software for Computing and Annotating Genomic Ranges. PLOS Computational Biology, 2013. 9(8): p. e1003118.

Lee, H.-K., et al. (2016). Three Dimensional Human Neuro-Spheroid Model of Alzheimer's Disease Based on Differentiated Induced Pluripotent Stem Cells. PLoS One 11, e0163072.

Lee, J. H., and Gleeson, J. G. (2010). The role of primary cilia in neuronal function. Neurobiol. Dis. 38, 167-172.

Lee, Y. J., et al. Inflammation and Alzheimer's disease. Arch Pharm Res 33, 1539-1556 (2010).

Lewis, P., et al. (2001). Expression of BRI- amyloid beta peptide fusion proteins: a novel method for specific high-level expression of amyloid beta peptides. Biochim. Biophys. Acta 1537,58-62.

Ling, D., et al. (2014). Accumulation of Amyloid-Like Aβ 1-42 in AEL (Autophagy-Endosomal-Lysosomal) Vesicles: Potential Implications for Plaque Biogenesis. ASN Neuro 6, AN20130044.

Ling, D. & Salvaterra, P. M. in Alzheimer's Dis. Pathog. Concepts, Shifting Paradig. Ther. Targets (Monte, S. D. La) (InTech, 2011).

Ling, D. & Salvaterra, P. M. M. Brain aging and Aβ₁₋₄₂ neurotoxicity converge via deterioration in autophagy-lysosomal system: a conditional Drosophila model linking Alzheimer's neurodegeneration with aging. Acta Neuropathol. 121,183-91 (2011).

Ling, D., et al. (2009). Abeta42-Induced Neurodegeneration via an Age-Dependent Autophagic-Lysosomal Injury in Drosophila. PLoS One 4, e4201.

Mahley, R. W. & Huang, Y. Apolipoprotein E Sets the Stage: Response to Injury Triggers Neuropathology. Neuron 76,871-885 (2012).

Maury, Y. et al. Combinatorial analysis of developmental cues efficiently converts humanpluripotent stem cells into multiple neuronal subtypes. Nat. Biotechnol. 33,89-96 (2014).

McGowan, et al. (2005). Aβ42 Is Essential for Parenchymal and Vascular Amyloid Deposition in Mice. Neuron 47,191-199.

Mortazavi, A., et al., Mapping and quantifying mammalian transcriptomes by RNA-Seq. Nat Meth, 2008. 5(7): p. 621-628.

Müller, U. C., and Zheng, H. (2013). Physiological functions of APP family proteins. Cold Spring Harb. Perspect. Med. 2,1-18.

Mungenast, A. E., et al. (2016). Modeling Alzheimer's disease with human induced pluripotent stem (iPS) cells. Mol. Cell. Neurosci. 73,13-31.

Muratore, C. R., et al. (2014). The familial Alzheimer's disease APPV717I mutation alters APP processing and Tau expression in iPSC-derived neurons. Hum. Mol. Genet. 23, 3523-3536.

Muratore, C. R., et al. (2017). Cell-type Dependent Alzheimer's Disease Phenotypes: Probing the Biology of Selective Neuronal Vulnerability. Stem Cell Reports 9,1868-1884.

Musiek, E. S., and Holtzman, D. M. (2015). Three dimensions of the amyloid hypothesis: time, space and “wingmen.” Nat. Neurosci. 18, 800-806. doi:10.1038/nn.4018.

Nilsson, P., et al. (2013). Aβ Secretion and Plaque Formation Depend on Autophagy. Cell Rep. 7, 1-9.

Nixon, R. A. (2017). Amyloid precursor protein and endosomal-lysosomal dysfunction in Alzheimer's disease: inseparable partners in a multifactorial disease. FASEB J. 31, 2729-2743.

Nixon, R. A., and Yang, D.-S. S. (2012). Autophagy and Neuronal Cell Death in Neurological Disorders. Cold Spring Harb Perspect Biol 4, 1-23.

Park, J., et al. (2018). A 3D human triculture system modeling neurodegeneration and neuroinflammation in Alzheimer's disease. Nat. Neurosci. 21, 941-951.

Pollio, G., et al. (2008). Increased expression of the oligopeptidase THOP1 is a neuroprotective response to Aβ toxicity. Neurobiol. Dis. 31, 145-158.

Poteryaev, D., et al. (2010). Identification of the switch in early-to-late endosome transition. Cell 141, 497-508.

Raja, W. K., et al. (2016). Self- Organizing 3D Human Neural Tissue Derived from Induced Pluripotent Stem Cells Recapitulate Alzheimer's Disease Phenotypes. PLoS One 11, e0161969.

Re, D. B. et al. Necroptosis Drives Motor Neuron Death in Models of Both Sporadic and Familial ALS. Neuron 81, 1001-1008 (2014).

Robinson, M. D., et al., edgeR: a Bioconductor package for differential expression analysis of digital gene expression data. Bioinformatics, 2010. 26(1): p. 139-140.

Saghatelian, A., et al. (2004). Activity-based probes for the proteomic profiling of metalloproteases. Proc. Natl. Acad. Sci. 101, 10000-10005.

Saito, T., et al. (2014). Single App knock-in mouse models of Alzheimer's disease. Nat. Neurosci. 17, 1-4.

Sasaguri, H., et al. (2017). APP mouse models for Alzheimer's disease preclinical studies. EMBO J. 36, 2473-2487.

Schindelin, J., et al. (2012). Fiji: an open-source platform for biological-image analysis. Nat. Methods 9, 676-682.

Schluter, O. M. (2004). A Complete Genetic Analysis of Neuronal Rab3 Function. J. Neurosci. 24, 6629-6637.

Selkoe, D. J., and Hardy, J. (2016). The amyloid hypothesis of Alzheimer's disease at 25 years. EMBO Mol. Med. 8, 1-14.

Shakes, L. A., et al. (2012). Using BAC transgenesis in zebrafish to identify regulatory sequences of the amyloid precursor protein gene in humans. BMC Genomics 13, 451.

Shaltouki, A., et al. Efficient Generation of Astrocytes from Human Pluripotent Stem Cells in Defined Conditions. Stem Cells 31, 941-952 (2013).

Shi, Y., et al. (2012). A human stem cell model of early Alzheimer's disease pathology in Down syndrome. Sci Transl Med 4, 124ra29- 124ra29.

Smith, K., et al. (2015). CIDRE: an illumination- correction method for optical microscopy. Nature Methods, 12(5), 404-406.

Teplow, D. B. (2013). On the subject of rigor in the study of amyloid p-protein assembly. Alzheimers. Res. Ther. 5, 39.

van Helmond, Z., et al. (2010). Oligomeric Aβ in Alzheimer's Disease: Relationship to Plaque and Tangle Pathology, APOE Genotype and Cerebral Amyloid Angiopathy. Brain Pathol. 20, 468-480.

Villeneuve, S., et al. The potential applications of Apolipoprotein E in personalized medicine. Front. Aging Neurosci. 6, 1-11 (2014).

Walsh, D. M., et al. (2000). The oligomerization of amyloid-protein begins intracellularly in cells derived from human brain. Biochemistry 39, 10831-10839.

Warden, C., et al., Optimal calculation of RNA-seq fold-change values. Int. J. Comput. Bioinformatics In Silico Model., 2013. 2: p. 285-292

Wicklund, L., et al. (2010). B-Amyloid 1-42 Oligomers Impair Function of Human Embryonic Stem Cell-Derived Forebrain Cholinergic Neurons. PLoS One 5, e15600.

Woodruff, G., et al. (2013). The Presenilin-1 ΔE9 Mutation Results in Reduced γ-Secretase Activity, but Not Total Loss of PS1 Function, in Isogenic Human Stem Cells. Cell Rep. 5, 974-985.

Yagi, T. et al. Modeling familial Alzheimer's disease with induced pluripotent stem cells. Hum Mol Genet 20, 4530-4539 (2011).

Yamin, R., et al. (1999). Metalloendopeptidase EC 3.4.24.15 Is Necessary for Alzheimer' s Amyloid- NL Peptide Degradation*. Biochemistry.

Young, J. E., and Goldstein, L. S. B. (2012). Alzheimer's disease in a dish: promises and challenges of human stem cell models. Hum. Mol. Genet. 21, R82-R89.

Young, M. D., et al., Gene ontology analysis for RNA-seq: accounting for selection bias. Genome Biology, 2010. 11(2): p. 1- 12.

Yu, J.-T., et al. Apolipoprotein E in Alzheimer's Disease: An Update. Annu. Rev. Neurosci. 37, 79-100 (2014).

Yuan, J., et al. (2003). Diversity in the Mechanisms of Neuronal Cell Death. Neuron 40, 401-413.

Zahs, K. R. et al. ‘Too much good news’—are Alzheimer mouse models trying to tell us how to prevent, not cure, Alzheimer's disease? Trends Neurosci. 33, 381-389 (2010).

Zhang, B., et al. (2013). Integrated systems approach identifies genetic nodes and networks in late-onset Alzheimer's disease. Cell 153, 707-20.

Zheng, H., et al. (1995). beta-Amyloid precursor protein-deficient mice show reactive gliosis and decreased locomotor activity. Cell 81, 525-531.

1. A recombinant stem cell comprising:
a modified amyloid beta precursor protein (APP) gene, wherein the modified APP gene comprises an amyloid β peptide (Aβ) coding sequence, and wherein the recombinant embryonic stem cell directly expresses an Aβ encoded by the Aβ coding sequence.

2. The recombinant stem cell of claim 1, wherein the Aβ coding sequence is an Amyloid Beta₁₋₄₀ (Aβ₁₋₄₀) coding sequence or an Amyloid Beta₁₋₄₂ (A₁₋₄₂) coding sequence.

3. The recombinant stem cell of claim 1, wherein the modified APP gene further comprises a secretory signal sequence.

4. The recombinant stem cell according to claim 1, further comprising a modified Apolipoprotein E (APOE) gene that confers a genotype selected from APOE2/E2, APOE3/E3, or APOE4/E4.

5. (canceled)

6. The recombinant stem cell according to claim 1, further comprising a lineage-related reporter gene.

7. The recombinant stem cell according to claim 6, wherein the lineage-related reporter gene comprises a sequence that encodes a cell type marker selected from the group consisting of: a neuronal marker, a dendritic marker, an axonal marker, an astrocyte marker, a glial marker, and a microglial marker, and a fluorescent reporter.

8. (canceled)

9. (canceled)

10. The recombinant stem cell according to claim 7, wherein the fluorescent reporter is a fluorescent protein gene selected from the group consisting of: RFP, CFP, YFP, mCherry, AcGFP1, DsRed-Monomer, tdTomato, DsRed, DsRed-Express, and E2-Crimson.

11. A population of recombinant neuronal cells comprising:
a population of differentiated human cells of a desired cell type, wherein the population of cells comprise a modified amyloid beta precursor protein (APP) gene, wherein the modified APP gene comprises an amyloid β peptide (Aβ) coding sequence, and wherein the recombinant neuronal cells directly expresses an Aβ encoded by the Aβ coding sequence.

12. The population of recombinant neuronal cells of claim 11, wherein the Aβ coding sequence is an Amyloid Beta₁₋₄₀ (Aβ₁₋₄₀) coding sequence or an Amyloid Beta₁₋₄₂ (Aβ₁₋₄₂) coding sequence.

13. The population of recombinant neuronal cells of claim 11, wherein the modified APP gene further comprises a secretory signal sequence.

14. The population of recombinant neuronal cells of claim 11, wherein the desired cell type is a motor neuron, a cholinergic motor neuron, or a CNS non-limb innervating neuron.

15. The population of recombinant neuronal cells according to claim 11, wherein the population of cells is isogenic.

16. The population of recombinant neuronal cells of claim 11, wherein the population of recombinant neuronal cells are derived from a population of recombinant stem cells subjected to at least one differentiation protocol designed to favor generating neurons selected from the group consisting of: motor neurons, cholinergic motor neurons, and CNS non-limb innervating neurons, or to favor generating astrocytes or microglia.

17-19. (canceled)

20. The population of recombinant neuronal cells according to claim 11, wherein the population of recombinant neuronal cells is co-cultured with cells from another source.

21. The population of recombinant neuronal cells according to claim 11, wherein the population of recombinant neuronal cells is co-cultured with at least one other population of cells; wherein the at least one other population of cells is subjected to a different differentiation protocol than the population of recombinant neuronal cells.

22. A method for screening for one or more therapeutic agents for treatment of a neurodegenerative condition, the method comprising:
measuring an initial level of Aβ₁₋₄₂ produced by a population of recombinant cells, wherein the population of cells comprises a modified amyloid beta precursor protein (APP) gene which comprises an amyloid β peptide (Aβ) coding sequence, and wherein the recombinant cells directly expresses an Aβ encoded by the Aβ coding sequence;
contacting the population of cells with a candidate therapeutic agent; and
measuring a second level of Aβ₁₋₄₂ produced by the population of cells after contacting the population with the candidate therapeutic agent; and
identifying the agent as a candidate for treating the neurodegenerative condition when the second level of Aβ₁₋₄₂ is lower than the initial level,
wherein the one or more therapeutic agents are selected from the group consisting of drugs, salts, minerals, antibodies, humanized antibodies, enzymes, proteins, peptides, cells, modified cells, stem cells, plant-based substances, plant derivatives, antioxidants, and antioxidant derivatives.

23. The method of claim 22, wherein the Aβ coding sequence is an Amyloid Beta₁₋₄₀ (Aβ₁₋₄₀) coding sequence or an Amyloid Beta₁₋₄₂ (Aβ₁₋₄₂) coding sequence.

24. The method of claim 22, wherein the modified APP gene further comprises a secretory signal sequence.

25. The method of claim 22, wherein the population of cells has undergone a differentiation protocol.

26. The method of claim 22, wherein the population of cells is co-cultured with at least one other population of cells.

27-28. (canceled)


SEQUENCE LISTING (46 sequences)

SEQ ID NO: 1 - DNA, 18 bp, Artificial Sequence
  TALEN 1
cgcctggctctgagcccc

SEQ ID NO: 2 - DNA, 15 bp, Artificial Sequence
  TALEN 2
gccgaggaaactgac

SEQ ID NO: 3 - DNA, 18 bp, Artificial Sequence
  TALEN 3
cgggctccgtcagtttcc

SEQ ID NO: 4 - DNA, 15 bp, Artificial Sequence
  TALEN 4
cctccgcgtgctctc

SEQ ID NO: 5 - DNA, 49 bp, Artificial Sequence
  Rat preproenkephain secretory signal
atggcgcagttcctgagactttgcatctggctcgtagcgcttgggtcct

SEQ ID NO: 6 - DNA, 23 bp, Artificial Sequence
  Rat preproenkephain secretory signal
gcctcctggctacagtgcaggca

SEQ ID NO: 7 - DNA, 33 bp, Artificial Sequence
  Abeta42
gatgcagaattccgacatgactcaggatatgaa

SEQ ID NO: 8 - DNA, 50 bp, Artificial Sequence
  Abeta42
gttcatcatcaaaaattggtgttctttgcagaagatgtgggttcaaacaa

SEQ ID NO: 9 - DNA, 43 bp, Artificial Sequence
  Abeta42
aggtgcaatcattggactcatggtgggcggtgttgtcatagcg

SEQ ID NO: 10 - DNA, 28 bp, Artificial Sequence
  Abeta40
gatgcagaattccgacatgactcaggat

SEQ ID NO: 11 - DNA, 51 bp, Artificial Sequence
  Abeta40
atgaagttcatcatcaaaaattggtgttctttgcagaagatgtgggttcaa

SEQ ID NO: 12 - DNA, 47 bp, Artificial Sequence
  Abeta40
acaaaggtgcaatcattggactcatggtgggcggtgttgtcatagcg

SEQ ID NO: 13 - DNA, 27 bp, Artificial Sequence
  5′ Recombinant forward primer
gaagtaaatgggttggccgcttctttg

SEQ ID NO: 14 - DNA, 26 bp, Artificial Sequence
  5′ Recombinant reverse primer
cctgagtcatgtcggaattctgcatc

SEQ ID NO: 15 - DNA, 19 bp, Artificial Sequence
  3′ Recombinant forward primer
gtcgaggtgcccgaaggac

SEQ ID NO: 16 - DNA, 20 bp, Artificial Sequence
  3′ Recombinant reverse primer
gggcaacgattcaagagcga

SEQ ID NO: 17 - DNA, 31 bp, Artificial Sequence
  LHA forward primer
ttaacgcggccgcggttcgttctaaagatag

SEQ ID NO: 18 - DNA, 30 bp, Artificial Sequence
  LHA reverse primer
agctgcagagatctagtcagctgatccggc

SEQ ID NO: 19 - DNA, 30 bp, Artificial Sequence
  RHA forward primer
aatatctgcagggtaccagcggtaggcgag

SEQ ID NO: 20 - DNA, 27 bp, Artificial Sequence
  RHA reverse primer
atttctcgagtgctccttcccccttcc

SEQ ID NO: 21 - DNA, 24 bp, Artificial Sequence
  GAPDH forward primer
atggggaaggtgaaggtcggagtc

SEQ ID NO: 22 - DNA, 28 bp, Artificial Sequence
  GAPDH reverse primer
gggatttccattgatgacaagcttcccg

SEQ ID NO: 23 - DNA, 18 bp, Artificial Sequence
  Edited Rat SS-Abeta forward primer
atggcgcagttcctgaga

SEQ ID NO: 24 - DNA, 23 bp, Artificial Sequence
  Edited Rat SS-Abeta reverse primer
atgattgcacctttgtttgaacc

SEQ ID NO: 25 - DNA, 18 bp, Artificial Sequence
  Edited Rat SS-Abeta42 forward primer
atggcgcagttcctgaga

SEQ ID NO: 26 - DNA, 17 bp, Artificial Sequence
  Edited Rat SS-Abeta42 reverse primer
cgctatgacaacaccgc

SEQ ID NO: 27 - DNA, 20 bp, Artificial Sequence
  APP Exon 1/2 forward primer
gtttggcactgctcctgctg

SEQ ID NO: 28 - DNA, 25 bp, Artificial Sequence
  APP Exon 1/2 reverse primer
ctgatggatctgaatcccacttccc

SEQ ID NO: 29 - DNA, 24 bp, Artificial Sequence
  APP Exon 5/6 forward primer
gaggaggatgactcggatgtctgg

SEQ ID NO: 30 - DNA, 26 bp, Artificial Sequence
  APP Exon 5/6 reverse primer
ggtggttctctctgtggcttcttcgt

SEQ ID NO: 31 - DNA, 17 bp, Artificial Sequence
  APP Exon 16/17 forward primer
gatgcagaattccgaca

SEQ ID NO: 32 - DNA, 21 bp, Artificial Sequence
  APP Exon 16/17 reverse primer
ccatgatgaatggatgtgtac

SEQ ID NO: 33 - DNA, 23 bp, Artificial Sequence
  RAB5 forward primer
ctagtgcttcgttttgtgaaagg

SEQ ID NO: 34 - DNA, 24 bp, Artificial Sequence
  RAB5 reverse primer
catatacaactatggctgcttgtg

SEQ ID NO: 35 - DNA, 22 bp, Artificial Sequence
  LAMP1 forward primer
ctactgctgttgctgctgctcg

SEQ ID NO: 36 - DNA, 23 bp, Artificial Sequence
  LAMP1 reverse primer
ggcatctgatggcaggtcaaagg

SEQ ID NO: 37 - DNA, 27 bp, Artificial Sequence
  LC3B forward primer
atgccgtcggagaagaccttcaagcag

SEQ ID NO: 38 - DNA, 33 bp, Artificial Sequence
  LC3B reverse primer
cttgatgagctcactcatgttgacatggtcagg

SEQ ID NO: 39 - DNA, 21 bp, Artificial Sequence
  VAMP7 forward primer
ctggaggtgacagagcagatt

SEQ ID NO: 40 - DNA, 20 bp, Artificial Sequence
  VAMP7 reverse primer
tgtctgtgctcttgaaccgt

SEQ ID NO: 41 - DNA, 21 bp, Artificial Sequence
  RAB11 forward primer
gacgacgagtacgactacctc

SEQ ID NO: 42 - DNA, 22 bp, Artificial Sequence
  RAB11 reverse primer
gcaaactctactccaatggtgc

SEQ ID NO: 43 - DNA, 21 bp, Artificial Sequence
  PICALM forward primer
agcaagtacatggggagatgc

SEQ ID NO: 44 - DNA, 20 bp, Artificial Sequence
  PICALM reverse primer
ttaaggccagctgaagggtg

SEQ ID NO: 45 - DNA, 18 bp, Artificial Sequence
  RAB3A forward primer
aaagtcaccgccgctagg

SEQ ID NO: 46 - DNA, 20 bp, Artificial Sequence
  RAB3A reverse primer
accttgaagtcgatgcccac